(19) United States
(12) Patent Application Publication
(10) Pub. No.: US 2025/0147049 A1
(43) Pub. Date: May 8, 2025

(54) METHODS FOR DETECTING CSF TAU SPECIES WITH STAGE AND PROGRESSION OF ALZHEIMER'S DISEASE, AND USE THEREOF

(71) Applicant: Washington University, St. Louis, MO (US)

(72) Inventors: Randall Bateman, St. Louis, MO (US); Eric McDade, St. Louis, MO (US); Nicolas Barthelemy, St. Louis, MO (US); Kanta Horie, St. Louis, MO (US); Yan Li, St. Louis, MO (US)

(21) Appl. No.: 18/273,701

(22) PCT Filed: Jan. 21, 2022
(86) PCT No.: PCT/US2022/013409

§ 371 Date: Jul. 21, 2023

Related U.S. Application Data
(60) Provisional application No. 63/140,203, filed on Jan. 21, 2021.
(60) Provisional application No. 63/151,051, filed on Feb. 18, 2021.
(60) Provisional application No. 63/170,185, filed on Apr. 2, 2021.
(60) Provisional application No. 63/180,915, filed on Apr. 28, 2021.
(60) Provisional application No. 63/187,697, filed on May 12, 2021.
(60) Provisional application No. 63/213,006, filed on Jun. 21, 2021.

(51) Int. Cl. G01N 33/68; G01N 1/40; G01N 33/53

(52) U.S. Cl. CPC G01N 33/6896; G01N 1/4044; G01N 33/53; G01N 2333/46; G01N 2440/14; G01N 2800/2821; G01N 2800/52; G01N 2800/56

(57) ABSTRACT

The present disclosure provides methods to quantify and analyze various CSF Tau species and the use thereof to measure pathological features and/or clinical symptoms of tauopathies, including determining the amount of time to dementia due to Alzheimer's disease, determining the time from dementia onset, staging Alzheimer's disease, guiding treatment decisions, and evaluate the clinical efficacy of certain therapeutic interventions.

### CROSS REFERENCE TO RELATED APPLICATIONS

This application claims priority of U.S. Provisional application No. 63/140,203, filed Jan. 21, 2021, U.S. Provisional application No. 63/151,051, filed Feb. 18, 2021, U.S. Provisional application No. 63/170,185, filed Apr. 2, 2021, U.S. Provisional application No. 63/180,915, filed Apr. 28, 2021, U.S. Provisional application No. 63/187,697, filed May 12, 2021, and U.S. Provisional application No. 63/213,006, filed Jun. 21, 2021, each of which is hereby incorporated by reference in its entirety.

### GOVERNMENTAL RIGHTS

This invention was made with government support under AG046363 and AG032438 awarded by the National Institutes of Health. The government has certain rights in the invention.

### REFERENCE TO A SEQUENCE LISTING

This application contains a Sequence Listing that has been submitted in ASCII format via EFS-Web and is hereby incorporated by reference in its entirety. The ASCII copy, created on Jan. 20, 2022, is named “715616_ST25.txt”, and is 8,936 bytes in size.

### FIELD OF THE TECHNOLOGY

The present disclosure encompasses methods to quantify and analyze various CSF Tau species and the use thereof to measure pathological features and/or clinical symptoms of tauopathies, including primary tauopathies (e.g. MAPT, PSP, CBD) and secondary tauopathies (e.g. AD due to Aβ amyloidosis).

### BACKGROUND OF THE DISCLOSURE

The microtubule-associated protein tau (MAPT or tau) plays an essential role in the morphology and physiology of neurons. Tau has six different isoforms of the full-length protein and undergoes a number of possible post-translational modifications including acetylation, glycosylation and phosphorylation.

Accumulation of tau protein as insoluble aggregates in the brain is one of the hallmarks of Alzheimer's disease and other neurodegenerative diseases called tauopathies. Intracellular tangles in the cerebral cortex are defining pathological feature of Alzheimer disease (AD) and correlate with the onset of clinical symptoms long after the appearance of extracellular amyloid-β (Aβ) plaques, which begin to develop up to two decades before symptom onset. Tau pathology appears to propagate across brain regions and spread by the transmission of specific pathological tau species from cell to cell in a prion-like manner although the nature of these species (i.e., monomeric, oligomeric, and fibril species) and the spreading process are uncertain.

In AD, soluble p-tau and unphosphorylated tau are increased by two-fold in cerebrospinal fluid. It has been proposed that these changes reflect the effects of neuronal death (neurodegeneration) passively releasing tau and NFT into the CSF. However, in other tauopathies with significant NFT pathology and neurodegeneration (e.g., progressive supranuclear palsy, frontotemporal lobar degeneration-tau), CSF levels of soluble p-tau and total tau do not increase. These observations suggest that Aβ may trigger a process that leads to the unique tauopathy of AD, an idea that is supported by cellular and animal models. This concept is further supported by an increase in the active production of soluble tau in the presence of amyloid plaques in humans.

Several mass spectrometry (MS) studies suggest that the microtubule-binding region (MTBR) of tau is enriched in aggregates in AD brain. Moreover, a series of cryogenic electron microscopy (Cryo-EM) studies demonstrate that the core structure of tau aggregates consists of a subsegment of the MTBR domain and the particular conformation depends upon the tauopathy. However, these studies used postmortem brain tissue and little is known about the pathophysiology of corresponding extracellular MTBR-containing tau species in biological samples such as CSF.

Although tau comprises a hall mark AD pathology and can be measured in aggregated or soluble forms, important gaps remain in our understanding of how the post-translational modifications and isoforms of this critical neuronal protein lead to the development of NFT and neurodegeneration in humans. For example, it is unknown what, if any, pathophysiologic changes occur to tau during the preclinical and clinical stages of AD. As such, it is unclear to what extent, if any, tau can be used to stage subjects prior to onset of symptoms associated with AD and guide treatment decisions.

### SUMMARY

In an aspect, the present disclosure encompasses a method for measuring time to dementia onset in a subject by (ai) measuring phosphorylation occupancy at residue T205 of tau and measuring MTBR-tau299/MTBR-tau354 ratio, and optionally measuring MTBR-tau212, phosphorylation occupancy at residue T217 MTBR-tau243, MTBR-tau3R, or a combination thereof, in a blood sample or a CSF sample obtained from the subject, or (aii) measuring phosphorylation occupancy at residue T205 of tau, measuring phosphorylation occupancy at residue T217 of tau, and measuring MTBR-tau212, in a blood sample or a CSF sample obtained from the subject; and (b) using the measurements of (ai) or (aii) to calculate time to dementia onset, wherein time to dementia onset is time in years to a Clinical Dementia Rating greater than zero.

In another aspect, the present disclosure encompasses method for measuring time to dementia onset in a subject without cognitive or behavioral symptoms of Alzheimer's disease, the method generally includes (a) processing a blood sample or a CSF sample from the subject to obtain a first population of tau species and a depleted sample, and then processing the depleted sample to obtain a second population of tau species, wherein the first population of tau species is enriched for N-terminal tau and/or mid-domain tau, and wherein the second population of enriched tau species is enriched for MTBR-tau; (bi) measuring phosphorylation occupancy at residue T205 of tau in the first population of tau species and measuring MTBR-tau299/MTBR-tau354 ratio in the second population of tau species, and optionally measuring MTBR-tau212 in the second population of tau species, or
(bii) measuring phosphorylation occupancy at residue T205 of tau and measuring phosphorylation occupancy at residue T217 of tau in the first population of tau species, and measuring MTBR-tau212 in the second population of tau species; and
(c) calculating time to dementia onset using the measurements of (bi) or (bii), wherein time to dementia onset is time in years to a Clinical Dementia Rating greater than zero.

In some embodiments, processing a blood sample or a CSF sample from the subject to obtain a first population of enriched tau species and a depleted sample comprises contacting the blood sample or the CSF sample with an epitope-binding agent that specifically binds to an epitope within the N-terminus of tau, or contacting the blood sample or the CSF sample with an epitope-binding agent that specifically binds to an epitope within the mid-domain of tau, or contacting the blood sample or the CSF sample with a first epitope-binding agent that specifically binds to an epitope within the N-terminus of tau and with a second epitope-binding agent that specifically binds to an epitope within the mid-domain of tau, wherein the first and second epitope-binding agents are used sequentially or at the same time; optionally wherein the epitope-binding agent that specifically binds to an epitope within the N-terminus of tau is HJ8.5 or another epitope-binding agent that specifically binds the same epitope as HJ8.5, and optionally wherein the epitope-binding agent that specifically binds to an epitope within the mid-domain of tau is Tau1 or another epitope-binding agent that specifically binds the same epitope as Tau1.

In some embodiments, processing the depleted sample to obtain a second population of enriched tau species comprises performing a chemical extraction step to enrich for MTBR-tau species, optionally wherein the chemical extract step comprises admixing an acid to precipitate proteins of the depleted sample, optionally wherein the acid is perchloric acid, and wherein the MTBR-tau species are in the supernatant after removal of the precipitated proteins; or contacting the depleted sample with an epitope-binding agent that specifically binds to at least one epitope within the MTBR of tau, optionally wherein the epitope-binding agent is 77G7, RD3, RD4, UCB1017, or PT76 described in Vandermeeren et al., J Alzheimers Dis, 2018, 65:265-281, or 7G6 described in Roberts et al., Acta Neuropathol Commun, 2020, 8: 13, or antigen-binding fragments of 77G7, RD3, RD4, UCB1017, PT76, or 7G6, or other epitope-binding agents that specifically bind the same epitopes as 77G7, RD3, RD4, UCB1017, PT76, or7G6.

In some embodiments, processing a blood sample or a CSF sample from the subject to obtain a first population of enriched tau species and a depleted sample includes contacting the blood sample or the CSF sample with an epitope-binding agent the specifically binds to an epitope within the N-terminus of tau, or contacting the blood sample or the CSF sample with an epitope-binding agent the specifically binds to an epitope within the mid-domain of tau, or contacting the blood sample or the CSF sample with a first epitope-binding agent that specifically binds to an epitope within the N-terminus of tau and with a second epitope-binding agent that specifically binds to an epitope within the mid-domain of tau, wherein the first and second epitope-binding agents are used sequentially or at the same time, optionally wherein the epitope-binding agent that specifically binds to an epitope within the N-terminus of tau is HJ8.5 or another epitope-binding agent that specifically binds the same epitope as HJ8.5, and optionally wherein the epitope-binding agent that specifically binds to an epitope within the mid-domain of tau is Tau1 or another epitope-binding agent that specifically binds the same epitope as Tau1; and processing the depleted sample to obtain a second population of enriched tau species comprises performing a chemical extraction step to enrich for MTBR-tau species, optionally wherein the chemical extract step comprises admixing an acid to precipitate proteins of the depleted sample, optionally wherein the acid is perchloric acid, and wherein the MTBR-tau species are in the supernatant after removal of the precipitated proteins; or contacting the depleted sample with an epitope-binding agent that specifically binds to at least one epitope within the MTBR of tau, optionally wherein the epitope-binding agent is 77G7, RD3, RD4, UCB1017, or PT76 described in Vandermeeren et al., J Alzheimers Dis, 2018, 65:265-281, or 7G6 described in Roberts et al., Acta Neuropathol Commun, 2020, 8: 13, or antigen-binding fragments of 77G7, RD3, RD4, UCB1017, PT76, or 7G6, or other epitope-binding agents that specifically bind the same epitopes as 77G7, RD3, RD4, UCB1017, PT76, or 7G6.

In each of the above aspects, the subject may have a CDR of zero.

In some embodiments, the calculated time to dementia onset measurement can be used to stage a subject's disease progression, stage a subject's brain pathology or select a therapeutic agent or a diagnostic agent for a subject.

In still another aspect, the present disclosure provides a method for treating a subject without cognitive or behavioral symptoms of Alzheimer's disease by administering to the subject a therapeutic agent or the diagnostic agent wherein the therapeutic agent decreases Aβ production, prevents or antagonizes Aβ aggregation, or increases brain Aβ clearance, optionally wherein the therapeutic agent is a gamma-secretase inhibitor, a beta-secretase inhibitor, a passive immunotherapy (including but not limited to an anti-Aβ antibody, an anti-tau antibody, or an anti-ApoE antibody), or an active immunotherapy or wherein the therapeutic agent prevents or antagonizes tau aggregation, increases neurofilbrillary tangle clearance, alters tau phosphorylation patterns, optionally wherein the therapeutic agent is a tau protein aggregation inhibitor, a kinase inhibitor, a phosphatase activator, a passive immunotherapy (including but not limited to an anti-tau antibody), or an active immunotherapy.

In another aspect, the present disclosure provides a method for measuring time from dementia onset in a subject with cognitive or behavioral symptoms of Alzheimer's disease by (ai) measuring phosphorylation occupancy at residue T205 of tau and measuring MTBR-tau299/MTBR-tau354 ratio, and optionally measuring MTBR-tau212, phosphorylation occupancy at residue T217 MTBR-tau243, MTBR-tau3R, or a combination thereof, in a blood sample or a CSF sample obtained from the subject, or (aii) measuring phosphorylation occupancy at residue T205 of tau, measuring phosphorylation occupancy at residue T217 of tau, and measuring MTBR-tau212, in a blood sample or a CSF sample obtained from the subject; and (b) using the measurements of (ai) or (aii) to calculate time from dementia onset, wherein time from dementia onset is time in years from a Clinical Dementia Rating greater than zero.

In yet another aspect, the present disclosure provides method for measuring time from dementia onset in a subject with cognitive or behavioral symptoms of Alzheimer's disease by (a) processing a blood sample or a CSF sample from the subject to obtain a first population of tau species and a depleted sample, and then processing the depleted sample to obtain a second population of tau species, wherein the first population of tau species is enriched for N-terminal tau and/or mid-domain tau, and wherein the second population of enriched tau species is enriched for MTBR-tau; (bi) measuring phosphorylation occupancy at residue T205 of tau in the first population of tau species and measuring MTBR-tau299/MTBR-tau354 ratio in the second population of tau species, and optionally measuring MTBR-tau212 in the second population of tau species, or (bii) measuring phosphorylation occupancy at residue T205 of tau and measuring phosphorylation occupancy at residue T217 of tau in the first population of tau species, and measuring MTBR-tau212 in the second population of tau species; and (c) calculating time from dementia onset using the measurements of (bi) or (bii), wherein time from dementia onset is time in years from a Clinical Dementia Rating greater than zero.

In some embodiments, processing a blood sample or a CSF sample from the subject to obtain a first population of enriched tau species and a depleted sample includes contacting the blood sample or the CSF sample with an epitope-binding agent the specifically binds to an epitope within the N-terminus of tau, or contacting the blood sample or the CSF sample with an epitope-binding agent the specifically binds to an epitope within the mid-domain of tau, or contacting the blood sample or the CSF sample with a first epitope-binding agent that specifically binds to an epitope within the N-terminus of tau and with a second epitope-binding agent that specifically binds to an epitope within the mid-domain of tau, wherein the first and second epitope-binding agents are used sequentially or at the same time; optionally wherein the epitope-binding agent that specifically binds to an epitope within the N-terminus of tau is HJ8.5 or another epitope-binding agent that specifically binds the same epitope as HJ8.5, and optionally wherein the epitope-binding agent that specifically binds to an epitope within the mid-domain of tau is Tau1 or another epitope-binding agent that specifically binds the same epitope as Tau1.

In some embodiments, processing the depleted sample to obtain a second population of enriched tau species comprises
performing a chemical extraction step to enrich for MTBR-tau species,
optionally wherein the chemical extract step comprises admixing an acid to precipitate proteins of the depleted sample, optionally wherein the acid is perchloric acid, and wherein the MTBR-tau species are in the supernatant after removal of the precipitated proteins; or
contacting the depleted sample with an epitope-binding agent that specifically binds to at least one epitope within the MTBR of tau,
optionally wherein the epitope-binding agent is 77G7, RD3, RD4, UCB1017, or PT76 described in Vandermeeren et al., J Alzheimers Dis, 2018, 65:265-281, or 7G6 described in Roberts et al., Acta Neuropathol Commun, 2020, 8: 13, or antigen-binding fragments of 77G7, RD3, RD4, UCB1017, PT76, or 7G6, or other epitope-binding agents that specifically bind the same epitopes as 77G7, RD3, RD4, UCB1017, PT76, or7G6.

The method of claim 18, wherein
processing a blood sample or a CSF sample from the subject to obtain a first population of enriched tau species and a depleted sample comprises
contacting the blood sample or the CSF sample with an epitope-binding agent the specifically binds to an epitope within the N-terminus of tau, or
contacting the blood sample or the CSF sample with an epitope-binding agent the specifically binds to an epitope within the mid-domain of tau, or
contacting the blood sample or the CSF sample with a first epitope-binding agent that specifically binds to an epitope within the N-terminus of tau and with a second epitope-binding agent that specifically binds to an epitope within the mid-domain of tau, wherein the first and second epitope-binding agents are used sequentially or at the same time,
optionally wherein the epitope-binding agent that specifically binds to an epitope within the N-terminus of tau is HJ8.5 or another epitope-binding agent that specifically binds the same epitope as HJ8.5, and optionally wherein the epitope-binding agent that specifically binds to an epitope within the mid-domain of tau is Tau1 or another epitope-binding agent that specifically binds the same epitope as Tau1; and processing the depleted sample to obtain a second population of enriched tau species comprises performing a chemical extraction step to enrich for MTBR-tau species, optionally wherein the chemical extract step comprises admixing an acid to precipitate proteins of the depleted sample, optionally wherein the acid is perchloric acid, and wherein the MTBR-tau species are in the supernatant after removal of the precipitated proteins; or contacting the depleted sample with an epitope-binding agent that specifically binds to at least one epitope within the MTBR of tau, optionally wherein the epitope-binding agent is 77G7, RD3, RD4, UCB1017, or PT76 described in Vandermeeren et al., J Alzheimers Dis, 2018, 65:265-281, or 7G6 described in Roberts et al., Acta Neuropathol Commun, 2020, 8: 13, or antigen-binding fragments of 77G7, RD3, RD4, UCB1017, PT76, or 7G6, or other epitope-binding agents that specifically bind the same epitopes as 77G7, RD3, RD4, UCB1017, PT76, or7G6.

In some embodiments, the calculated time to dementia onset measurement is used to stage a subject's disease progression, to stage a subject's brain pathology, or to select a therapeutic agent or a diagnostic agent for a subject.

Accordingly, the present disclosure provides methods for treating a subject without cognitive or behavioral symptoms of Alzheimer's disease, the method comprising administering to the subject the therapeutic agent or the diagnostic agent.

In another aspect, the present disclosure provides, a method for measuring change in cognition in a subject, the method comprising (a) measuring phosphorylation occupancy at one or more residue of tau selected from T111, T153, T181, T217 and T231 in a blood sample or a CSF sample obtained from the subject, and measuring at least one of MTBR-tau275, MTBR-tau299, and MTBR-3R in a blood sample or a CSF sample obtained from the subject, and optionally measuring total tau in a blood sample or a CSF sample obtained from the subject; and (b) using the measurements of (a) to calculate a change in cognition wherein the change in cognition is equivalent to the change in cognition measured by cognitive composite score consisting of the delayed recall score from the International Shopping List Test, the Logical Memory delayed recall score from the Wechsler Memory Scale-Revised, the Digit Symbol Coding test total score from the Wechsler Adult Intelligence Scale-Revised, and the MMSE total score.

In some embodiments, the measurement of change in cognition is used to evaluate the effectiveness of a therapeutic agent.

Other aspects and iterations of the disclosure are described more thoroughly below.

### BRIEF DESCRIPTION OF THE FIGURES

The application file contains at least one photograph executed in color. Copies of this patent application publication with color photographs will be provided by the Office upon request and payment of the necessary fee.

FIG. 1 is a schematic of the longest human tau isoform (2N4R). The N-terminus (N term), mid domain, MTBR, and C-terminus (C term) are identified for this isoform and will vary in a predictable way for other tau isoforms (e.g., 2N3R, 1NR4, 1N3R, 0N4R, and 0N3R).

FIG. 2A is a schematic illustrating several methods of the present disclosure. The method detailed within the blue box (right—Tau-Chemical extraction method) is one method. The combination of the red box (left—IP for N-terminal Tau and mid-domain tau) and the blue box (right Tau-Chemical extraction method) is another method.

FIG. 2B is a schematic illustrating several methods of the present disclosure. The method detailed within the blue box (right Tau-Chemical extraction method) is one method. The combination of the red box (left IP for N-terminal Tau and mid-domain tau) and the blue box (right Tau-Chemical extraction method) is another method.

FIG. 3 is a schematic illustrating a method of the present disclosure.

FIG. 4 is a schematic illustrating a method of the present disclosure.

FIG. 5 is an illustration showing tau pathology evolves through distinct phases in Alzheimer Disease. Measuring four different soluble tau species and insoluble tau in a group of participants with deterministic Alzheimer disease mutations we show over the course of 35 years (x-axis) tau related changes unfold (y-axis) and differ based on the stage of disease and other measurable biomarkers. Starting with the development of fibrillar amyloid pathology phosphorylation at position 217 (purple) and 181 (blue) begins to increase. With the increase in neuronal dysfunction (based metabolic changes) phosphorylation at position 205 (green) begins to increase along with soluble tau (orange). Lastly, with the onset of neurodegeneration (based on brain atrophy and cognitive decline) tau PET tangles (red) begin to develop while phosphorylation of 217 and 181 begins to decrease. Together, this highlights the dynamic and diverging patterns of soluble and aggregated tau over the course of the disease and close relationship with amyloid pathology.

FIG. 6A and FIG. 6B show CSF MTBR-tau 299, MTBR-tau306, and MTBR-tau 354 exhibit distinct characteristics in whole Alzheimer's disease continuum, reflecting the tangles status. FIG. 6A is an illustration of 4R and 3R tau showing regions of E2814 binding. FIG. 6B show the concentration of MTBR-tau299, MTBR-tau306, and MTBR tau-354 in relation to estimated years to symptom onset. Red points: symptomatic mutation carrier (MC); orange points: asymptomatic MC; blue points: non-carrier (NC); Red curve” loess curve for MC; blue curve: loess curve for NC.

FIG. 7 is an illustration for the secretion of MTBR-tau species into CSF from brain tau aggregates across clinical Alzheimer's disease stage. In preclinical AD stages, brain tau aggregates are immature such that the three MTBR-tau species (MTBR-tau-243, MTBR-tau-299, and MTBR-tau-354) are secreted into CSF in equal concentrations. However, as tau aggregates mature with disease progression and form a rigid core of MTBR-tau-354 (R4 domain), MTBR-tau-354 species in the CSF stabilizes. The MTBR-tau-299 (R2 to R3 domain) species joins the rigid core structure at later symptomatic stages, while the MTBR-tau-243 species (upstream of R1 domain) remains exposed, enabling protease digestion and release into CSF. Eventually, the imbalance for these three species in CSF is observed as a reflection of brain tau aggregate formation.

FIG. 8A, FIG. 8B, and FIG. 8C show ROC and AUC of pTau species in classifying amyloid PET status. FIG. 8A shows pT111, pT153, pS208, and pT231 for mutation carriers. FIG. 8B shows pT111, pT153, pS208, and pT231 for both mutation carriers and non-carriers. FIG. 8C shows the area under the curve and 95% confidence interval for the groups.

FIG. 9A, FIG. 9B, and FIG. 9C show ROC and AUC of MTBR-tau species in classifying amyloid PET status. FIG. 9A shows MTBR-tau212, MTBR-tau243, MTBR-tau260, and MTBR-tau275 for mutation carriers. FIG. 9B shows MTBR-tau212, MTBR-tau243, MTBR-tau260, and MTBR-tau275 for both mutation carriers and non-carriers. FIG. 9C shows the area under the curve and 95% confidence interval for the groups.

FIG. 10A, FIG. 10B, and FIG. 10C show ROC and AUC of MTBR-tau species in classifying amyloid PET status. FIG. 10A shows MTBR-tau282, MTBR-tau299, MTBR-tau306, and MTBR-tau354 for mutation carriers. FIG. 10B shows MTBR-tau282, MTBR-tau299, MTBR-tau306, and MTBR-tau354 for both mutation carriers and non-carriers. FIG. 10C shows the area under the curve and 95% confidence interval for the groups.

FIG. 11A, FIG. 11B, and FIG. 11C show ROC and AUC of MTBR-tau species in classifying amyloid PET status. FIG. 11A shows MTBR-tau386, MTBR-tau396, MTBR-tau299/MTBR-tau354 ratio, and MTBR-tau299/MTBR-tau282 ratio for mutation carriers. FIG. 11B shows MTBR-tau386, MTBR-tau396, MTBR-tau299/MTBR-tau354 ratio, and MTBR-tau299/MTBR-tau282 for both mutation carriers and non-carriers. FIG. 11C shows the area under the curve and 95% confidence interval for the groups.

FIG. 12A, FIG. 12B, FIG. 12C, FIG. 12D, FIG. 12E, and FIG. 12F show box plots of pT111, pT153 and pS208 in mutation carriers. FIG. 12A shows pT111/T111 original value phosphorylation ratios increase by PiB quartiles (n=47 for Q1, 46 for Q2, 46 for Q3, and 47 for Q4). FIG. 12B shows pT153/T153 original values of phosphorylation ratios increase by PiB quartiles (n=47 for Q1, 46 for Q2, 46 for Q3, and 47 for Q4). FIG. 12C shows pS208/S208 original values of phosphorylation ratios increase by PiB quartiles (n=47 for Q1, 46 for Q2, 46 for Q3, and 47 for Q4). FIG. 12D shows pT111/T111 standardized value phosphorylation ratios increase by PiB quartiles (n=47 for Q1, 46 for Q2, 46 for Q3, and 47 for Q4). FIG. 12E shows pT153/T153 standardized values of phosphorylation ratios increase by PiB quartiles (n=47 for Q1, 46 for Q2, 46 for Q3, and 47 for Q4). FIG. 12F shows pS208/S208 standardized values of phosphorylation ratios increase by PiB quartiles (n=47 for Q1, 46 for Q2, 46 for Q3, and 47 for Q4). P-values are from Wilcoxon rank sum test.

FIG. 13A, FIG. 13B, FIG. 13C, FIG. 13D, FIG. 13E, FIG. 13F, FIG. 13G, FIG. 13H, FIG. 13I, FIG. 13J, FIG. 13K, FIG. 13L, FIG. 13M, FIG. 13N, FIG. 13O, FIG. 13P, FIG. 13Q, FIG. 13R and FIG. 13S show the concentrations of various tau species in relation to estimated years to symptom onset. FIG. 13A shows pT111/T111; FIG. 13B shows pT153/T153; FIG. 13C shows pS208/S208; FIG. 13D shows pT231/T231; FIG. 13E shows pT153; FIG. 13F shows pS208; FIG. 13G shows pT231; FIG. 13H shows pT231/T231; FIG. 13I shows MTBR-tau212; FIG. 13J shows MTBR-tau 243; FIG. 13K shows MTBR-tau260; FIG. 13L shows MTBR-tau275; FIG. 13M shows MTBR-tau282; FIG. 13N shows MTBR-tau299; FIG. 13O shows MTBR-tau3R; FIG. 13P shows MTBR-tau354; FIG. 13Q shows MTBR-tau386; FIG. 13R shows MTBR-tau396; FIG. 13S shows MTBR-tau299/MTBR-tau282; FIG. 13T shows MTBR-tau299/MTBR-tau354 ratio.

FIG. 14A, FIG. 14B, FIG. 14C, FIG. 14D, FIG. 14E, FIG. 14F, FIG. 14G, FIG. 14H, FIG. 14I, FIG. 14J, FIG. 14K, FIG. 14L, FIG. 14M, FIG. 14N, FIG. 14O, and FIG. 14P show annual change of pTau & MTBR by DIAN EYO (annual changes were estimated from linear mixed effects models). EYO cutoff for cross sectional data: −19 for pT111/T111, −22 for pT153/T153, −22 for pS208/S208; EYO cutoff for rate of change: −25 for pT111/T111 and pT153/T153, no cutoff for pS208/S208; All the longitudinal analysis were based on log transformed pT111/T111 and pS208/S208, and square root transformed pT153/T153. FIG. 14A shows pT111/T111; FIG. 14B shows pT153/T153; FIG. 14C shows pS208/S208; FIG. 14D shows pT231/T231; FIG. 14E shows MTBR-tau212; FIG. 14F shows MTBR-tau243; FIG. 14G shows MTBR-tau260; FIG. 14H shows MTBR-tau275; FIG. 14I shows MTBR-tau282; FIG. 14J shows MTBR-tau 299; FIG. 14K shows MTBR-tau3R; FIG. 14L shows MTBR-tau299/MTBR-tau282; FIG. 14M shows MTBR-tau299/MTBR-tau354; FIG. 14N shows MTBR-tau354; FIG. 14O shows MTBR-tau386; FIG. 13P shows MTBR-tau396.

FIG. 15 shows association between baseline biomarkers and the longitudinal rate of change of cognitive composite.

FIG. 16A, FIG. 16B, FIG. 16C, FIG. 16D, FIG. 16E, FIG. 16F, FIG. 16G, FIG. 16H, FIG. 16I, FIG. 16J, FIG. 16K, FIG. 16L, FIG. 16M, FIG. 16N, FIG. 16O, and FIG. 16P show the association in annual change between various tau species and global cognition. FIG. 16A shows pT111/T111; FIG. 16B shows pT153/T153; FIG. 16C shows pS208/S208; FIG. 16D shows pT231/T231; FIG. 16E shows MTBR-tau212; FIG. 16F shows MTBR-tau243; FIG. 16G shows MTBR-tau260; FIG. 16H shows MTBR-tau275; FIG. 16I shows MTBR-tau282; FIG. 16J shows MTBR-tau299; FIG. 16K shows MTBR-tau3R; FIG. 16L shows MTBR-tau299/MTBR-tau282; FIG. 16M shows MTBR-tau299/MTBR-tau354; FIG. 16N shows MTBR-tau354; FIG. 16O shows MTBR-tau386; FIG. 16P shows MTBR-tau396. pT153/T153 and MTBR 3R have higher correlation in annual change than other tau species

FIG. 17A and FIG. 17B show the correlation of phosphorylation occupancy at various sites of tau phosphorylation for mutation carriers (asymptomatic and symptomatic) at baseline and annual change. FIG. 17A shows the correlation of pT111/T111, pT153/T153 and pS208/208 with pT217/217. FIG. 17B shows the correlation of pT111/T111, pT153/T153 and pS208/208 with pT205/205.

FIG. 18A, FIG. 18B, FIG. 18C, FIG. 18D, FIG. 18E, FIG. 18F, FIG. 18G, FIG. 18H, FIG. 18I, FIG. 18J, and FIG. 18K show heatmaps for the correlations in baseline and annual rate of change of mutation carriers (symptomatic and asymptomatic) and non-carriers. FIG. 18A shows a heatmap for the correlations in baseline of MCs. All MTBR species and total tau clustered together (several small clusters); ratios from MTBR dataset is clustered with other tau species instead of those in MTBR. pS202/S202 is not associated with any other tau species; TPPSS (specify the region of the total tau). FIG. 18B shows a heatmap for the correlations in baseline using all MC. FIG. 18C shows a heatmap for the correlations in baseline using asymptomatic MC. FIG. 18D shows a heatmap for the correlations in baseline using symptomatic MC. FIG. 18E shows a heatmap for the correlations in baseline using non-carriers. FIG. 18F shows a heatmap where color represents the correlation between annual rate of change of two markers for mutation carriers. FIG. 18G shows a heatmap where color represents the correlation between annual rate of change of two markers for asymptomatic mutation carriers. FIG. 18H shows a heatmap where color represents the correlation between annual rate of change of two markers for symptomatic mutation carriers. FIG. 18I shows a heatmap where color represents the absolute value of correlation between annual rate of change of two markers for mutation carriers. FIG. 18J shows a heatmap where color represents the absolute value of correlation between annual rate of change of two markers for asymptomatic mutation carriers. FIG. 18K shows a heatmap where color represents the absolute value of correlation between annual rate of change of two markers for symptomatic mutation carriers.

FIG. 19A, FIG. 19B, and FIG. 19C show the predication of DIAN EYO in all mutation carriers, asymptomatic mutation carrier, and symptomatic mutation carriers. Semi-partial R square indicates model R-square is added by x if VAR1 is included in the model. Squared Semipartial correlation indicates variable importance because it measures incremental value in R-Square. Semi-partial R square won't add up to R square as the total variation in dependent variable also constitutes a portion that is due to within correlations between independent variables. FIG. 19A shows the prediction of DIAN EYO for pT205/T205, MTBR-tau212, and MTBR-tau299/MTBR-tau354 in all mutation carriers.

FIG. 19B shows the predication of DIAN EYO of pT205/T205, pT217/T217 and MTBR-tau212 in asymptomatic mutation carriers. FIG. 19C shows the predication of DIAN EYO of pT205/T205 in symptomatic mutation carriers.

FIG. 20A and FIG. 20B show tau species abnormal rate by every 5 EYO interval. 95 percentile of each biomarker in NC were used as the threshold to define each biomarker in MC as normal and abnormal. FIG. 20A is a table showing tau species abnormal rate by every 5 EYO interval. FIG. 20B is a line graph showing tau species abnormal rate by every 5 EYO interval.

FIG. 21A shows the comparisons of the effect size of the annual rate of change (mean over standard deviation ration-MSR).

FIG. 21B shows comparisons of the effect size of the annual rate of change (mean over standard deviation ratio—MSR). aMC and basline EYO≥-25.

FIG. 22A and FIG. 22B show MTBR-tau299 and MTBR-tau354 change is inflected at AD onset (CDR=1). FIG. 22A shows MTBR-tau299. FIG. 22B shows MTBR-tau534.

FIG. 23 shows MTBR-tau299/MTBR-tau354 ratio boosts the discrimination power for AD staging.

FIG. 24A and FIG. 24B show CSF MTBR-299/354 ratio performance to predict cognitive scores in whole AD continuum. FIG. 24A shows MTBR-299/354 and CDR-SB. FIG. 24B shows MTBR-299/354 and MMSE.

FIG. 25 shows a summary table MTBR-tau vs Cognitive scores.

FIG. 26A shows correlation between MTBR-tau299/354 ratio and pT217% occupancy.

FIG. 26B shows pT217% is well correlated with E2814-associated MTBR-tau299/354 which recapitulates “Early stage tau pathology”->pT217% may be used as surrogate efficacy marker for E2814 clinical trials.

FIG. 27A is a schematic of trypti peptides from tau (grey bars) that were quantified and discussed in FIG. 27B and FIG. 27C.

FIG. 27B, and FIG. 27C are graphs showing brain MTBR tau species comprising MTBR tau-243, 299 and 354 are enriched in aggregated Alzheimer's disease brain insoluble extracts compared to control brain extracts, confirming that MTBR tau is specifically deposited in Alzheimer's disease brain. The graphs show the enrichment profile of tau peptides from (FIG. 18B) control and Alzheimer's disease brains (n=2 with six—eight brain regions samples/group in discovery cohort) and (FIG. 18C) from control (amyloid-negative, n=8), very mild to moderate Alzheimer's disease (AD) (amyloid-positive, CDR=0.5-2, n=5), and severe AD brains (amyloid-positive, CDR=3, n=7) (total n=20 in validation cohort). The relative abundance of tau peptides was quantified relative to the mid-domain (residue 181-190) peptide for internal normalization. The species containing the upstream region of microtubule binding region (MTBR) domain (residue 243-254, MTBR tau-243) and repeat region 2 (R2) to R3 and R4 (residues 299-317, MTBR tau-299 and 354-369, MTBR tau-354, respectively) were highly enriched in the insoluble fraction from Alzheimer's disease brains compared to controls and were specifically enriched by clinical stage of disease progression as measured by the CDR. MTBR tau-299 and MTBR tau-354 are located inside the filament core, whereas MTBR tau-243 is located outside the core of Alzheimer's disease aggregates (Fitzpatrick et al., 2017). Of note, residue 195-209 was decreased in Alzheimer's disease brains, potentially due to a high degree of phosphorylation. Data are represented as box-and-whisker plots with Tukey method describing median, interquartile interval, minimum, maximum, and individual points for outliers. Significance in statistical test: ****p<0.001, ***p<0.001, **p<0.01, *p<0.05.

FIG. 28A is a schematic of tryptic peptides from tau (grey bars) that were quantified in Example 3, and further discussed in FIG. 19B and FIG. 19C, as well as the general binding site of the antibodies HJ8.5 and Tau1.

FIG. 28B is a graph showing the tau profile in control human CSF. Tau peptides in control human CSF from a cross-sectional cohort of amyloid-negative and CDR=0 patients (n=30) were quantified by Tau1/HJ8.5 immunoprecipitation focusing on N-terminal to mid-domain tau. To quantify the species containing the microtubule binding region (MTBR) and C-terminal region, post-immunoprecipitated CSF samples were chemically extracted and analyzed sequentially. Using the Tau1/HJ8.5 immunoprecipitation method (blue circle), peptide recovery dramatically decreased after reside 222; therefore, only N-terminal to mid-domain tau (residues 6-23 to 243-254) peptides were quantified by this method (Sato et al., 2018). In contrast, the chemical extraction method of post-immunoprecipitated CSF (red square) enabled quantification of whole regions of tau including the MTBR to C-terminal regions at concentrations between 0.4-7 ng/mL. Data are represented as means.

FIG. 29A, FIG. 29B, and FIG. 29C show CSF MTBR-tau-243, 299, and 354 species exhibit distinct characteristics in whole Alzheimer's disease continuum, reflecting the tangles status. FIG. 29A MTBR-tau-243, FIG. 29B MTBR-tau-299, and FIG. 29C MTBR-tau-354 concentrations. Amyloid-negative CDR=0 (n=30), amyloid-positive CDR=0 (n=18), amyloid-positive CDR=0.5 (n=28), amyloid-positive CDR 1 (n=12), and amyloid-negative CDR 0.5 (n=12). MTBR-tau-243 showed a continuous increase with AD progression through all clinical stages. MTBR-tau-299 and 354 concentrations similarly increased until very mild AD stage (amyloid-positive and CDR=0.5), but then either saturated (MTBR-tau-299) or decreased (MTBR-tau-354) at CDR 1. ****p<0.001, ***p<0.001, **p<0.01, *p<0.05. NS=not significant.

FIG. 30A, FIG. 30B, FIG. 30C, and FIG. 30D show CSF MTBR-tau-243 is the most highly correlated with tau PET measure in all tau species including p-tau217, suggesting CSF MTBR-tau-243 is the most promising biomarker to recapitulate tau pathology. Correlations between tau PET (AV-1451) SUVR and FIG. 30A MTBR-tau-243, FIG. 30B MTBR-tau-299, FIG. 30C p-tau217 concentrations and FIG. 30D p-tau217 phosphorylation occupancy in CSF (control n=15 and Alzheimer's disease (AD) n=20 from tau PET cohort). Open circle: control, filled squares: AD. MTBR-tau-243 showed the most significant correlation with tau PET SUVR (Spearman r=0.7588, p<0.0001), whereas MTBR-tau-299, p-tau217 concentrations and p-tau217 phosphorylation occupancy showed the moderate correlations (Spearman r=0.4584, 0.5478 and 0.5555, respectively) and the saturations were observed in high tau PET measures samples.

FIG. 31 shows MTBR-tau243 vs MTBR-tau212 by Chemical extraction “after Tau1/HJ8.1-IP where for MTBR-tau212, CX provides the staged increase while E2814-IP does not.

FIG. 32 shows MTBR-tau243 vs MTBR-tau212 by Chemical extraction “after Tau1/HJ8.1-IP where for MTBR-tau212, CX provides the staged increase while E2814-IP does not.

### DETAILED DESCRIPTION

Tau protein aggregation into neurofibrillary tangles in the central nervous system contributes to the etiology of certain neurodegenerative disorders, including Alzheimer's disease (AD). Though the mechanism of tau destabilization is not fully understood yet, tau protein has been found to be hyperphosphorylated in tau aggregates. In addition, the microtubule-binding region (MTBR) of tau has been suggested to be enriched in aggregates in AD brain. However, little is known about the pathophysiology of corresponding extracellular pTau and MTBR-containing tau species throughout the progression of AD. A plurality of tau peptides are present in blood and CSF, though detection and quantification of tau species in these biological samples has been hampered due to the very low abundance of these polypeptides.

Applicants have discovered that certain methods to quantify tau (e.g., phosphorylation at specific amino acid residues and/or MTBR tau) can be used to track the AD process across its preclinical asymptomatic stages to symptomatic stages. Given the extremely large variability in tau isoforms, post-translation modifications, abundance and solubility, the use of tau species to stage subjects prior to onset of symptoms associated with AD and guide treatment decisions has been elusive. However, Applicant has identified methods of quantifying specific combinations of tau species which are particularly useful for identifying years from onset of dementia due to AD and to the development of certain pathophysiological changes.

The methods disclosed herein employ unique combinations of processing steps that transform a biological sample into a sample suitable for quantifying various tau species. For instance, in some methods of the present disclosure, the processing steps deplete certain proteins while enriching for a plurality of tau proteins. In other methods of the present disclosure, the processing steps deplete certain proteins while enriching for a plurality of MTBR tau proteins. Certain methods disclosed herein are particularly suited for quantifying mid-domain-independent MTBR tau species. Also described herein are uses of mid-domain-independent MTBR tau species and tau phosphorylation at certain amino acid residues to measure clinical signs and symptoms of tauopathies, diagnose tauopathies, and direct treatment of tauopathies. These and other aspects and iterations of the invention are described more thoroughly below.

### I. Definitions

So that the present invention may be more readily understood, certain terms are first defined. Unless defined otherwise, all technical and scientific terms used herein have the same meaning as commonly understood by one of ordinary skill in the art to which embodiments of the invention pertain. Many methods and materials similar, modified, or equivalent to those described herein can be used in the practice of the embodiments of the present invention without undue experimentation, the preferred materials and methods are described herein. In describing and claiming the embodiments of the present invention, the following terminology will be used in accordance with the definitions set out below.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

An antibody, as used herein, refers to a complete antibody as understood in the art, i.e., consisting of two heavy chains and two light chains, and also to any antibody-like molecule that has an antigen binding region, including, but not limited to, antibody fragments such as Fab′, Fab, F(ab′)2, single domain antibodies, Fv, and single chain Fv. The term antibody also refers to a polyclonal antibody, a monoclonal antibody, a chimeric antibody and a humanized antibody. The techniques for preparing and using various antibody-based constructs and fragments are well known in the art. Means for preparing and characterizing antibodies are also well known in the art (See, e.g. Antibodies: A Laboratory Manual, Cold Spring Harbor Laboratory, 1988; herein incorporated by reference in its entirety).

As used herein, the term “aptamer” refers to a polynucleotide, generally a RNA or DNA that has a useful biological activity in terms of biochemical activity, molecular recognition or binding attributes. Usually, an aptamer has a molecular activity such as binging to a target molecule at a specific epitope (region). It is generally accepted that an aptamer, which is specific in it binding to a polypeptide, may be synthesized and/or identified by in vitro evolution methods. Means for preparing and characterizing aptamers, including by in vitro evolution methods, are well known in the art. See, for instance U.S. Pat. No. 7,939,313, herein incorporated by reference in its entirety.

The term “Aβ” refers to peptides derived from a region in the carboxy terminus of a larger protein called amyloid precursor protein (APP). The gene encoding APP is located on chromosome 21. There are many forms of Aβ that may have toxic effects: Aβ peptides are typically 37-43 amino acid sequences long, though they can have truncations and modifications changing their overall size. They can be found in soluble and insoluble compartments, in monomeric, oligomeric and aggregated forms, intracellularly or extracellularly, and may be complexed with other proteins or molecules. The adverse or toxic effects of Aβ may be attributable to any or all of the above noted forms, as well as to others not described specifically. For example, two such Aβ isoforms include Aβ40 and Aβ42; with the Aβ42 isoform being particularly fibrillogenic or insoluble and associated with disease states. The term “Aβ” typically refers to a plurality of Aβ species without discrimination among individual Aβ species. Specific Aβ species are identified by the size of the peptide, e.g., Aβ42, Aβ40, Aβ38 etc.

As used herein, the term “Aβ342/Aβ40 value” means the ratio of the amount of Aβ42 in a sample obtained from a subject compared to the amount of Aβ40 in the same sample.

“Aβ amyloidosis” is defined as clinically abnormal Aβ deposition in the brain. A subject that is determined to have Aβ amyloidosis is referred to herein as “amyloid positive,” while a subject that is determined to not have Aβ amyloidosis is referred to herein as “amyloid negative.” There are accepted indicators of Aβ amyloidosis in the art. At the time of this disclosure, Aβ amyloidosis is directly measured by amyloid imaging (e.g., PiB PET, fluorbetapir, or other imaging methods known in the art) or indirectly measured by decreased cerebrospinal fluid (CSF) Aβ42 or a decreased CSF Aβ42/40 ratio. [11C]PIB-PET imaging with mean cortical binding potential (MCBP) score >0.18 is an indicator of Aβ amyloidosis, as is cerebral spinal fluid (CSF) Aβ42 concentration of about 1 ng/ml measured by immunoprecipitation and mass spectrometry (IP/MS)). Alternatively, a cut-off ratio for CSF Aβ42/40 that maximizes the accuracy in predicting amyloid-positivity as determined by PIB-PET can be used. Values such as these, or others known in the art and/or used in the examples, may be used alone or in combination to clinically confirm Aβ amyloidosis. See, for example, Klunk W E et al. Ann Neurol 55(3) 2004, Fagan A M et al. Ann Neurol, 2006, 59(3), Patterson et. al, Annals of Neurology, 2015, 78(3): 439-453, or Johnson et al., J. Nuc. Med., 2013, 54(7): 1011-1013, each hereby incorporated by reference in its entirety. Subjects with Aβ amyloidosis may or may not be symptomatic, and symptomatic subjects may or may not satisfy the clinical criteria for a disease associated with Aβ amyloidosis. Non-limiting examples of symptoms associated with Aβ amyloidosis may include impaired cognitive function, altered behavior, abnormal language function, emotional dysregulation, seizures, dementia, and impaired nervous system structure or function. Diseases associated with Aβ amyloidosis include, but are not limited to, Alzheimer's Disease (AD), cerebral amyloid angiopathy (CAA), Lewy body dementia, and inclusion body myositis. Subjects with Aβ amyloidosis are at an increased risk of developing a disease associated with Aβ amyloidosis.

A “clinical sign of Aβ amyloidosis” refers to a measure of Aβ deposition known in the art. Clinical signs of Aβ amyloidosis may include, but are not limited to, Aβ deposition identified by amyloid imaging (e.g. PiB PET, fluorbetapir, or other imaging methods known in the art) or by decreased cerebrospinal fluid (CSF) Aβ42 or Aβ42/40 ratio. See, for example, Klunk W E et al. Ann Neurol 55(3) 2004, and Fagan A M et al. Ann Neurol 59(3) 2006, each hereby incorporated by reference in its entirety. Clinical signs of Aβ amyloidosis may also include measurements of the metabolism of Aβ, in particular measurements of Aβ42 metabolism alone or in comparison to measurements of the metabolism of other Aβ variants (e.g. Aβ37, Aβ38, Aβ39, Aβ40, and/or total Aβ), as described in U.S. patent Ser. Nos. 14/366,831, 14/523,148 and 14/747,453, each hereby incorporated by reference in its entirety. Additional methods are described in Albert et al. Alzheimer's & Dementia 2007 Vol. 7, pp. 170-179; McKhann et al., Alzheimer's & Dementia 2007 Vol. 7, pp. 263-269; and Sperling et al. Alzheimer's & Dementia 2007 Vol. 7, pp. 280-292, each hereby incorporated by reference in its entirety. Importantly, a subject with clinical signs of Aβ amyloidosis may or may not have symptoms associated with Aβ deposition. Yet subjects with clinical signs of Aβ amyloidosis are at an increased risk of developing a disease associated with Aβ amyloidosis.

A “candidate for amyloid imaging” refers to a subject that has been identified by a clinician as an individual for whom amyloid imaging may be clinically warranted. As a non-limiting example, a candidate for amyloid imaging may be a subject with one or more clinical signs of Aβ amyloidosis, one or more Aβ plaque associated symptoms, one or more CAA associated symptoms, or combinations thereof. A clinician may recommend amyloid imaging for such a subject to direct his or her clinical care. As another non-limiting example, a candidate for amyloid imaging may be a potential participant in a clinical trial for a disease associated with Aβ amyloidosis (either a control subject or a test subject).

An “Aβ plaque associated symptom” or a “CAA associated symptom” refers to any symptom caused by or associated with the formation of amyloid plaques or CAA, respectively, being composed of regularly ordered fibrillar aggregates called amyloid fibrils. Exemplary Aβ plaque associated symptoms may include, but are not limited to, neuronal degeneration, impaired cognitive function, impaired memory, altered behavior, emotional dysregulation, seizures, impaired nervous system structure or function, and an increased risk of development or worsening of Alzheimer's disease or CAA. Neuronal degeneration may include a change in structure of a neuron (including molecular changes such as intracellular accumulation of toxic proteins, protein aggregates, etc. and macro level changes such as change in shape or length of axons or dendrites, change in myelin sheath composition, loss of myelin sheath, etc.), a change in function of a neuron, a loss of function of a neuron, death of a neuron, or any combination thereof. Impaired cognitive function may include but is not limited to difficulties with memory, attention, concentration, language, abstract thought, creativity, executive function, planning, and organization. Altered behavior may include, but is not limited to, physical or verbal aggression, impulsivity, decreased inhibition, apathy, decreased initiation, changes in personality, abuse of alcohol, tobacco or drugs, and other addiction-related behaviors. Emotional dysregulation may include, but is not limited to, depression, anxiety, mania, irritability, and emotional incontinence. Seizures may include but are not limited to generalized tonic-clonic seizures, complex partial seizures, and non-epileptic, psychogenic seizures. Impaired nervous system structure or function may include, but is not limited to, hydrocephalus, Parkinsonism, sleep disorders, psychosis, impairment of balance and coordination. This may include motor impairments such as monoparesis, hemiparesis, tetraparesis, ataxia, ballismus and tremor. This also may include sensory loss or dysfunction including olfactory, tactile, gustatory, visual and auditory sensation. Furthermore, this may include autonomic nervous system impairments such as bowel and bladder dysfunction, sexual dysfunction, blood pressure and temperature dysregulation. Finally, this may include hormonal impairments attributable to dysfunction of the hypothalamus and pituitary gland such as deficiencies and dysregulation of growth hormone, thyroid stimulating hormone, lutenizing hormone, follicle stimulating hormone, gonadotropin releasing hormone, prolactin, and numerous other hormones and modulators.

As used herein, the term “subject” refers to a mammal, preferably a human. The mammals include, but are not limited to, humans, primates, livestock, rodents, and pets. A subject may be waiting for medical care or treatment, may be under medical care or treatment, or may have received medical care or treatment.

As used herein, the term “control population,” “normal population” or a sample from a “healthy” subject refers to a subject, or group of subjects, who are clinically determined to not have a tauopathy or Aβ amyloidosis, or a clinical disease associated with Aβ amyloidosis (including but not limited to Alzheimer's disease), based on qualitative or quantitative test results. A “normal” subject is usually about the same age as the individual to be evaluated, including, but not limited to, subjects of the same age and subjects within a range of 5 to 10 years.

As used herein, the term “blood sample” refers to a biological sample derived from blood, preferably peripheral (or circulating) blood. The blood sample can be whole blood, plasma or serum, although plasma is typically preferred.

The term “isoform”, as used herein, refers to any of several different forms of the same protein variants, arising due to alternative splicing of mRNA encoding the protein, post-translational modification of the protein, proteolytic processing of the protein, genetic variations and somatic recombination. The terms “isoform” and “variant” are used interchangeably.

The term “tau” refers to a plurality of isoforms encoded by the gene MAPT (or homolog thereof), as well as species thereof that are C-terminally truncated in vivo, N-terminally truncated in vivo, post-translationally modified in vivo, or any combination thereof. As used herein, the terms “tau” and “tau protein” and “tau species” may be used interchangeably. In many animals, including but not limited to humans, non-human primates, rodents, fish, cattle, frogs, goats, and chicken, tau is encoded by the gene MAPT. In animals where the gene is not identified as MAPT, a homolog may be identified by methods well known in the art.

In humans, there are six isoforms of tau that are generated by alternative splicing of exons 2, 3, and 10 of MAPT. These isoforms range in length from 352 to 441 amino acids. Exons 2 and 3 encode 29-amino acid inserts each in the N-terminus (called N), and full-length human tau isoforms may have both inserts (2N), one insert (1N), or no inserts (0N). All full-length human tau isoforms also have three repeats of the microtubule binding domain (called R). Inclusion of exon 10 at the C-terminus leads to inclusion of a fourth microtubule binding domain encoded by exon 10. Hence, full-length human tau isoforms may be comprised of four repeats of the microtubule binding domain (exon 10 included: R1, R2, R3, and R4) or three repeats of the microtubule binding domain (exon 10 excluded: R1, R3, and R4). Human tau may or may not be post-translationally modified. For example, it is known in the art that tau may be phosphorylated, ubiquinated, glycosylated, and glycated. Human tau also may or may not be proteolytically processed in vivo at the C-terminus, at the N-terminus, or at the C-terminus and the N-terminus. Accordingly, the term “human tau” encompasses the 2N3R, 2N4R, 1N3R, 1N4R, 0N3R, and 0N4R isoforms, as well as species thereof that are C-terminally truncated in vivo, N-terminally truncated in vivo, post-translationally modified in vivo, or any combination thereof. Alternative splicing of the gene encoding tau similarly occurs in other animals.

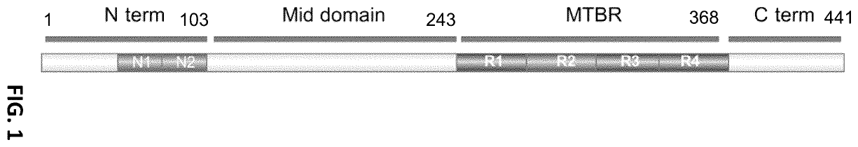

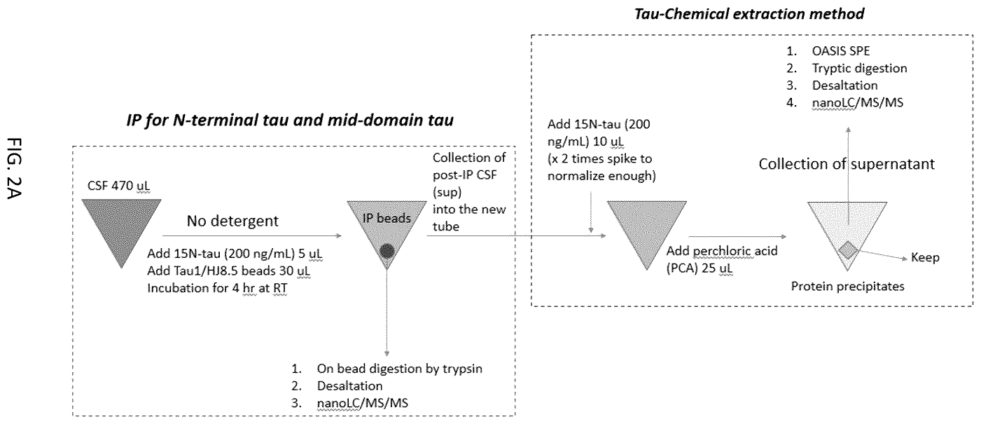

The term “tau-441,” as used herein, refers to the longest human tau isoform (2N4R), which is 441 amino acids in length. The amino acid sequence of tau-441 is provided as SEQ ID NO: 1. The N-terminus (N term), mid-domain, MTBR, and C-terminus (C term) are identified in FIG. 1 for this isoform. These regions will vary in a predictable way for other tau isoforms (e.g., 2N3R, 1NR4, 1N3R, 0N4R, and 0N3R). Accordingly, when amino acid positions are identified relative to tau-441, a skilled artisan will be able to determine the corresponding amino acid position for the other isoforms. Unless indicated otherwise amino acid residue numbering used in this disclosure is based on tau-441 (e.g., T217 is the threonine residue at position 217 of tau-441).

The term “N-terminal tau,” as used herein, refers to a tau protein, or a plurality of tau proteins, that comprise(s) two or more amino acids of the N-terminus of tau (e.g., amino acids 1-103 of tau-441, etc.).

The term “mid-domain tau,” as used herein, refers to a tau protein, or a plurality of tau proteins, that comprise(s) two or more amino acids of the mid-domain of tau (e.g., amino acids 104-243 of tau-441, etc.).

The term “MTBR tau,” as used herein, refers to a tau protein, or a plurality of tau proteins, that comprise(s) two or more amino acids of the microtubule binding region (MTBR) of tau (e.g., amino acids 244-368 of tau-441, etc.).

The term “C-terminal tau,” as used herein, refers to a tau protein, or a plurality of tau proteins, that comprise(s) two or more amino acids of the C-terminus of tau (e.g., amino acids 369-441 of tau-441, etc.).

A “proteolytic peptide of tau” refers to a peptide fragment of a tau protein produced by in vitro proteolytic cleavage. A “tryptic peptide of tau” refers to a peptide fragment of a tau protein produced by in vitro cleavage with trypsin. Tryptic peptides of tau may be referred to herein by their first four amino acids. For instance, “LQTA” (the first four amino acids of SEQ ID NO: 3) refers to the tryptic peptide LQTAPVPMPDLK (SEQ ID NO: 3). Non-limiting examples of other tryptic peptides identified by their first four amino acids include IGST (SEQ ID NO: 2), VQII (SEQ ID NO: 4), LDLS (SEQ ID NO: 5), HVPG (SEQ ID NO: 6), IGSL (SEQ ID NO: 7), VQIV (SEQ ID NO: 9), and TPPS (SEQ ID NO: 10).

A disease associated with tau deposition in the brain is referred to herein as a “tauopathy”. The term “tau deposition” is inclusive of all forms pathological tau deposits including but not limited to neurofibrillary tangles, neuropil threads, and tau aggregates in dystrophic neurites. Tauopathies known in the art include, but are not limited to, progressive supranuclear palsy (PSP), dementia pugilistica, chronic traumatic encephalopathy, frontotemporal dementia and parkinsonism linked to chromosome 17, Lytico-Bodig disease, Parkinson-dementia complex of Guam, tangle-predominant dementia, ganglioglioma and gangliocytoma, meningioangiomatosis, subacute sclerosing panencephalitis, lead encephalopathy, tuberous sclerosis, Hallervorden-Spatz disease, lipofuscinosis, Pick's disease, corticobasal degeneration (CBD), argyrophilic grain disease (AGD), Frontotemporal lobar degeneration (FTLD), Alzheimer's disease (AD), and frontotemporal dementia (FTD).

Tauopathies are classified by the predominance of tau isoforms found in the pathological tau deposits. Those tauopathies with tau deposits predominantly composed of tau with three MTBRs are referred to as “3R-tauopathies”. Pick's disease is a non-limiting example of a 3R-tauopathy. For clarification, pathological tau deposits of some 3R-tauopathies may be a mix of 3R and 4R tau isoforms with 3R isoforms predominant. Intracellular neurofibrillary tangles (i.e. tau deposits) in brains of subjects with Alzheimer's disease are generally thought to contain both approximately equal amounts of 3R and 4R isoforms. Those tauopathies with tau deposits predominantly composed of tau with four MTBRs are referred to as “4R-tauopathies”. PSP, CBD, and AGD are non-limiting examples of 4R-tauopathies, as are some forms of FTLD. Notably, pathological tau deposits in brains of some subjects with genetically confirmed FTLD cases, such as some V334M and R406W mutation carriers, show a mix of 3R and 4R isoforms.

A clinical sign of a tauopathy may be aggregates of tau in the brain, including but not limited to neurofibrillary tangles. Methods for detecting and quantifying tau aggregates in the brain are known in the art (e.g., tau PET using tau-specific ligands such as THK5317, THK5351, AV1451, PBB3, MK-6240, RO-948, PI-2620, GTP1, PM-PBB3, and JNJ64349311, JNJ-067), etc.).

The terms “treat,” “treating,” or “treatment” as used herein, refers to the provision of medical care by a trained and licensed professional to a subject in need thereof. The medical care may be a diagnostic test, a therapeutic treatment, and/or a prophylactic or preventative measure. The object of therapeutic and prophylactic treatments is to prevent or slow down (lessen) an undesired physiological change or disease/disorder. Beneficial or desired clinical results of therapeutic or prophylactic treatments include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented. Accordingly, a subject in need of treatment may or may not have any symptoms or clinical signs of disease.

The phrase “tau therapy” collectively refers to any imaging agent, therapeutic treatment, and/or a prophylactic or preventative measure contemplated for, or used with, subjects at risk of developing a tauopathy, or subjects clinically diagnosed as having a tauopathy. Non-limiting examples of imaging agents include functional imaging agents (e.g. fluorodeoxyglucose, etc.) and molecular imaging agents (e.g., Pittsburgh compound B, florbetaben, florbetapir, flutemetamol, radiolabeled tau-specific ligands, radionuclide-labeled antibodies, etc.). Non-limiting examples of therapeutic agents include cholinesterase inhibitors, N-methyl D-aspartate (NMDA) antagonists, antidepressants (e.g., selective serotonin reuptake inhibitors, atypical antidepressants, aminoketones, selective serotonin and norepinephrine reuptake inhibitors, tricyclic antidepressants, etc.), gamma-secretase inhibitors, beta-secretase inhibitors, anti-AP antibodies (including antigen-binding fragments, variants, or derivatives thereof), anti-tau antibodies (including antigen-binding fragments, variants, or derivatives thereof), stem cells, dietary supplements (e.g. lithium water, omega-3 fatty acids with lipoic acid, long chain triglycerides, genistein, resveratrol, curcumin, and grape seed extract, etc.), antagonists of the serotonin receptor 6, p38alpha MAPK inhibitors, recombinant granulocyte macrophage colony-stimulating factor, passive immunotherapies, active vaccines (e.g. CAD106, AF20513, etc.), tau protein aggregation inhibitors (e.g. TRx0237, methylthionimium chloride, etc.), therapies to improve blood sugar control (e.g., insulin, exenatide, liraglutide pioglitazone, etc.), anti-inflammatory agents, phosphodiesterase 9A inhibitors, sigma-1 receptor agonists, kinase inhibitors, phosphatase activators, phosphatase inhibitors, angiotensin receptor blockers, CB1 and/or CB2 endocannabinoid receptor partial agonists, p-2 adrenergic receptor agonists, nicotinic acetylcholine receptor agonists, 5-HT2A inverse agonists, alpha-2c adrenergic receptor antagonists, 5-HT 1A and 1D receptor agonists, Glutaminyl-peptide cyclotransferase inhibitors, selective inhibitors of APP production, monoamine oxidase B inhibitors, glutamate receptor antagonists, AMPA receptor agonists, nerve growth factor stimulants, HMG-CoA reductase inhibitors, neurotrophic agents, muscarinic M1 receptor agonists, GABA receptor modulators, PPAR-gamma agonists, microtubule protein modulators, calcium channel blockers, antihypertensive agents, statins, and any combination thereof.

“Significantly deviate from the mean” refers to values that are at least 1 standard deviation, preferably at least 1.3 standard deviations, more preferably at least 1.5 standard deviations or even more preferably at least 2 standard deviations, above or below the mean (i.e. the average level of tau species from a normal subject or normal population).

The phrase “Aβ and tau therapies” collectively refers to any imaging agent or therapeutic agent contemplated for, or used with, subjects at risk of developing Aβ amyloidosis or AD, subjects diagnosed as having Aβ amyloidosis, subjects diagnosed as having tauopathy, or subjects diagnosed as having AD.

### II. Methods for Measuring Tau

The present disclosure provides methods for measuring tau in a biological sample by mass spectrometry. Generally speaking, methods of the present disclosure for measuring tau in a biological sample comprise providing a biological sample, processing the biological sample by depleting one or more protein and then purifying tau, cleaving the purified tau with a protease and then optionally desalting the resultant cleavage product by solid phase extraction to obtain a sample comprising proteolytic peptides of tau, and performing liquid chromatography—mass spectrometry with the sample comprising proteolytic peptides of tau to detect and measure the concentration (relative or absolute) of at least one proteolytic peptide of tau. Thus, in practice, the disclosed methods use at least one proteolytic peptide of tau to detect and measure the amount of tau present in the biological sample.

In one example, a method of the present disclosure comprises (a) providing a biological sample selected from a blood sample or a CSF sample; (b) removing proteins from the biological sample by protein precipitation and separating the precipitated proteins to obtain a supernatant; (c) purifying tau from the supernatant by solid phase extraction; (d) cleaving the purified tau with a protease and then optionally desalting the resultant cleavage product by solid phase extraction to obtain a sample comprising proteolytic peptides of tau; and (e) performing liquid chromatography—mass spectrometry with the sample comprising proteolytic peptides of tau to detect and measure the concentration of at least one proteolytic peptide of tau.

In another example, a method of the present disclosure comprises (a) decreasing in a biological sample by affinity depletion N-terminal tau, mid-domain tau, or N-terminal tau and mid-domain tau, wherein the biological sample is a blood sample or a CSF sample; (b) enriching tau that remains after affinity depletion, which may be referred to as N-terminal-independent tau and/or mid-domain-independent tau, by a method that comprises (i) removing additional proteins from the biological sample by protein precipitation and separation of the precipitated proteins to obtain a supernatant, and then purifying tau from the supernatant by solid phase extraction, or (ii) affinity purifying MTBR tau, thereby producing by either (i) or (ii) enriched tau; (c) cleaving the enriched tau with a protease and then optionally desalting the resultant cleavage product by solid phase extraction to obtain a sample comprising proteolytic peptides of tau; and (d) performing liquid chromatography—mass spectrometry (LC/MS) with the sample comprising proteolytic peptides of tau to detect and measure the concentration of at least one proteolytic peptide of tau.

In another example, a method of present disclosure comprises (a) decreasing in a biological sample by affinity depletion N-terminal tau, mid-domain tau, or N-terminal tau and mid-domain tau, wherein the biological sample is a blood sample or a CSF sample; (b) removing additional proteins from the affinity depleted sample by protein precipitation and separation of the precipitated proteins to obtain a supernatant; (c) purifying tau from the supernatant by solid phase extraction; (d) cleaving the purified tau with a protease and then optionally desalting the resultant cleavage product by solid phase extraction to obtain a sample comprising proteolytic peptides of tau; and (e) performing liquid chromatography—mass spectrometry with the sample comprising proteolytic peptides of tau to detect and measure the concentration at least one proteolytic peptide of tau.

In another example, a method of the present disclosure comprises (a) decreasing in a biological sample by affinity depletion N-terminal tau, mid-domain tau, or N-terminal tau and mid-domain tau, wherein the biological sample is a blood sample or a CSF sample; (b) affinity purifying MTBR tau from the affinity depleted sample; (c) cleaving the purified MTBR tau with a protease and then optionally desalting the resultant cleavage product by solid phase extraction to obtain a sample comprising proteolytic peptides of MTBR tau; and (d) performing liquid chromatography—mass spectrometry with the sample comprising proteolytic peptides of MTBR tau to detect and measure the concentration at least one proteolytic peptide of MTBR tau.

In another example, a method of the present disclosure comprises (a) affinity purifying MTBR tau from a biological sample, wherein the biological sample is a blood sample or a CSF sample; (b) cleaving the purified MTBR tau with a protease and then optionally desalting the resultant cleavage product by solid phase extraction to obtain a sample comprising proteolytic peptides of MTBR tau; and (c) performing liquid chromatography—mass spectrometry with the sample comprising proteolytic peptides of MTBR tau to detect and measure the concentration at least one proteolytic peptide of MTBR tau.

The present disclosure further contemplates in each of the above methods measuring phosphorylation occupancy at one or more residue of tau. Phosphorylation occupancy, also referred to as the stoichiometry of phosphorylation, is measured by quantifying phosphorylation at one more residue of tau. Using residue T217 for illustration, phosphorylation occupancy is typically expressed pT217/T217, where the numerator “pT217” is an amount (relative or absolute) of phosphorylated residue T217 and the denominator “T217” is the amount (relative or absolute) or residue T217. When phosphorylation at two or more residues of tau is measured, the method may further comprise calculating a ratio or another mathematical relationship between the values. In some embodiments, methods herein comprise measuring tau phosphorylation at one or more residue chosen from T111, S113, T181, S199, S202, S208, T153, T175, T205, S214, T217, and T231. In some embodiments, methods herein comprise measuring tau phosphorylation at one or more residue chosen from T111, T181, S208, T153, T175, T205, S214, T217, and T231. In some embodiments, methods herein comprise measuring tau phosphorylation at one or more residue chosen from T111, T153, T181, T205, S208, T217, and T231. In some embodiments, methods herein comprise measuring tau phosphorylation at one or more residue chosen from T111, T153, T181, T205, T217, and T231. In some embodiments, methods herein comprise measuring tau phosphorylation at one or more residue chosen from T111, T153, T181, T217, and T231. In some embodiments, methods herein comprise measuring tau phosphorylation at one or more residue chosen from T111, T153, T181, T205, S208, and T217. In some embodiments, methods herein comprise measuring tau phosphorylation at one or more residue chosen from T181, T205, and T217. In some embodiments, methods herein comprise measuring tau phosphorylation at T205. In some embodiments, methods herein comprise measuring tau phosphorylation at T205 and optionally at one or more additional residue chosen from T111, T181, S208, T153, T175, S214, T217, and T231. In some embodiments, methods herein comprise measuring tau phosphorylation at T205 and optionally at one or more additional residue chosen from T111, T153, T181, S208, and T217. In some embodiments, methods herein comprise measuring tau phosphorylation at T205 and optionally at one or more additional residue chosen from T181, and T217.

Generally speaking, phosphorylation occupancy at one or more residue of tau may be measured using any sample comprising tau. However, the amino acid residue may influence which sample should be used. For instance, if a measurement of tau phosphorylation at T111 is desired and the blood or CSF sample was affinity-depleted of mid-domain tau using the antibody Tau1, or other affinity purification reagent that binds C-terminal to T111, then the tau bound to the affinity purification reagent should be used for the measurement of pT111/T111. In certain embodiments, measurements of phosphorylation occupancy may use a sample enriched for mid-domain tau (or enriched for N-terminal and mid-domain tau).

The present disclosure is not limited to any one particular method to quantitatively assess site-specific phosphorylation of tau. Suitable methods should discriminate tau isoforms that differ only in the phosphorylation status of a single amino acid, discriminate p-tau isoforms that are phosphorylated at different amino acids, and quantify changes in phosphorylation occurring at specific sites independently from the global change in total tau. Changes in phosphorylation stoichiometry occurring at specific sites independently from the global change in total tau may be quantified one of the three approaches: 1) relative comparison between phosphorylated peptide isomers, which can be used to estimate the relative abundance of each phosphorylated peptide sharing the same sequence; 2) normalizing phosphorylated peptides with any peptide from the tau protein as reference; and 3) absolute quantitation using internal synthetic labeled standards for each phosphorylated and non-phosphorylated peptide, where absolute quantitation values for each phosphorylated peptide is normalized with any absolute quantitation value obtained for any peptide from the tau protein. All three approaches use internal normalization for comparing relative phosphorylation changes for each site. Other methods known in the art may also be used. When using an internal synthetic labeled standard for absolute quantification, the labeled standard is preferably spiked into the sample prior to processing the sample to enrich for soluble tau.

In an exemplary embodiment, site-specific phosphorylation of tau is measured by high-resolution mass spectrometry. Suitable types of mass spectrometers are known in the art. These include, but are not limited to, quadrupole, time-of-flight, ion trap and Orbitrap, as well as hybrid mass spectrometers that combine different types of mass analyzers into one architecture (e.g., Orbitrap Fusion™ Tribrid™ Mass Spectrometer from ThermoFisher Scientific). Tau is typically proteolytically digested prior to MS analysis. Suitable proteases include, but are not limited to, trypsin, Lys-N, Lys-C, and Arg-N. When affinity purification/depletion is used to produce the tau sample, digestion may occur after eluting tau from the immobilized ligand or while tau is bound to the immobilized ligand. Affinity purification/depletion is described in detail in Section II(c). Following one or more clean-up steps, digested tau peptides may be separated by a liquid chromatography system interfaced with a high-resolution mass spectrometer. The chromatography system may be optimized by routine experimentation to produce a desired LC-MS pattern. A wide array of LC-MS techniques may be used to quantitatively analysis site-specific tau phosphorylation. Non-limiting examples include selected-reaction monitoring, parallel-reaction monitoring, selected-ion monitoring, and data-independent acquisition. As stated above, all quantitative assessments of site-specific tau phosphorylation should account for global changes in total tau. In an exemplary embodiment, a mass spectrometry protocol outlined in the Examples is used.

The present disclosure further contemplates in each of the above methods measuring total tau. Tau can be found in soluble and insoluble compartments, in monomeric and aggregated forms, in ordered or disordered structures, intracellularly and extracellularly, and may be complexed with other proteins or molecules. Accordingly, the source of the biological sample (e.g., brain tissue, CSF, blood, etc.) and any downstream processing of the biological sample will affect the totality of tau isoforms in a given sample. Total tau measurement can be performed by mass spectrometry. Alternatively, total tau can be measured by immunoassays or other method quantifying tau concentration. In a specific embodiment, total tau may be measured by mass spectrometry by quantifying the TPSL (the first four amino acids of SEQ ID NO: 18) tryptic peptide (i.e., TPSLPTPPTR (SEQ ID NO:18)) or the TPPS (the first four amino acids of SEQ ID NO: 10) tryptic peptide (i.e., TPPSSGEPPK (SEQ ID NO: 10)). In certain embodiments, measurements of total tau may use a sample enriched for mid-domain tau (or enriched for N-terminal and mid-domain tau).

In still further embodiments, the present disclosure contemplates in each of the above methods determining the presence/absence of one or more protein in the biological sample and/or measuring the concentration of one or more additional protein in the biological sample. In some embodiments, the one or more protein may be a protein depleted from the biological sample prior to purification of tau. For instance, in certain embodiments, N-terminal tau and/or mid-domain tau species may be identified and/or quantified separately from tau species (e.g., MTBR tau, C-terminal tau) quantified by the methods disclosed herein. Alternatively, or in addition, Aβ, ApoE, or any other protein of interest may be identified and/or quantified either by processing a portion of the biological sample in parallel, by depleting the protein of interest from the biological sample prior to utilization in the methods disclosed herein, or by depleting the protein of interest from the biological sample during the sample processing steps disclosed herein.

The biological sample, suitable internal standards, and the steps of depleting one or more protein, purifying tau, cleaving purified tau with a protease, and mass spectrometry are described in more detail below.

### (a) Biological Sample

Suitable biological samples include a blood sample or a cerebrospinal fluid (CSF) sample obtained from a subject. In some embodiments, the subject is a human. A human subject may be waiting for medical care or treatment, may be under medical care or treatment, or may have received medical care or treatment. In various embodiments, a human subject may be a healthy subject, a subject at risk of developing a neurodegenerative disease, a subject with signs and/or symptoms of a neurodegenerative disease, or a subject diagnosed with a neurodegenerative disease. In further embodiments, the neurodegenerative disease may be a tauopathy. In specific examples, the tauopathy may be Alzheimer's disease (AD), progressive supranuclear palsy (PSP), corticobasal degeneration (CBD), or frontotemporal lobar degeneration (FTLD). In other embodiments, the subject is a laboratory animal. In a further embodiment, the subject is a laboratory animal genetically engineered to express human tau and optionally one or more additional human protein (e.g., human Aβ, human ApoE, etc.).

CSF may have been obtained by lumbar puncture with or without an indwelling CSF catheter. Multiple blood or CSF samples contemporaneously collected from the subject may be pooled. Blood may have been collected by venipuncture with or without an intravenous catheter, or by a finger stick (or the equivalent thereof). Once collected, blood or CSF samples may have been processed according to methods known in the art (e.g., centrifugation to remove whole cells and cellular debris; use of additives designed to stabilize and preserve the specimen prior to analytical testing; etc.). Blood or CSF samples may be used immediately or may be frozen and stored indefinitely. Prior to use in the methods disclosed herein, the biological sample may also have been modified, if needed or desired, to include protease inhibitors, isotope labeled internal standards, detergent(s) and chaotropic agent(s), and/or to deplete other analytes (e.g. proteins peptides, metabolites).

The size of the sample used can and will vary depending upon the sample type, the health status of the subject from whom the sample was obtained, and the analytes to be analyzed (in addition to tau). CSF samples volumes may be about 0.01 mL to about 5 mL, or about 0.05 mL to about 5 mL. In a specific example, the size of the sample may be about 0.05 mL to about 1 mL CSF. Plasma sample volumes may be about 0.01 mL to about 20 mL.

### (b) Isotope-Labeled, Internal Tau Standard

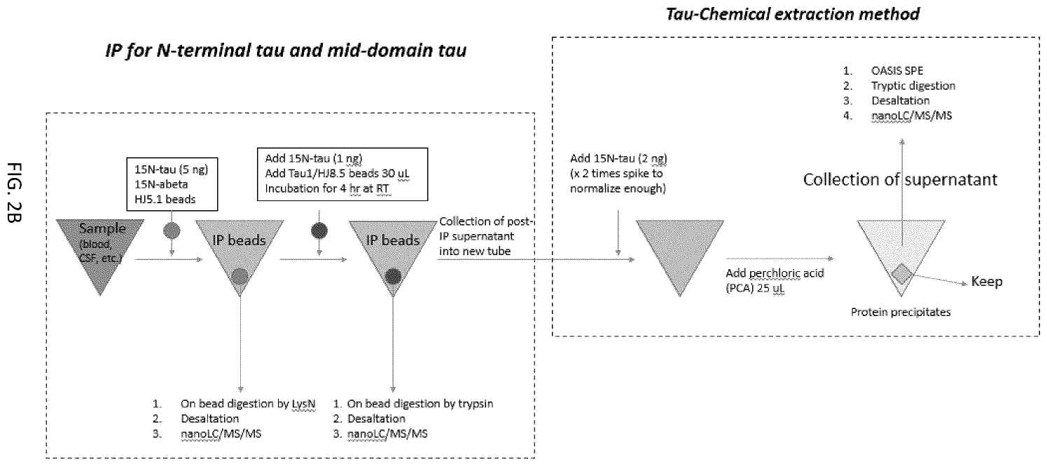

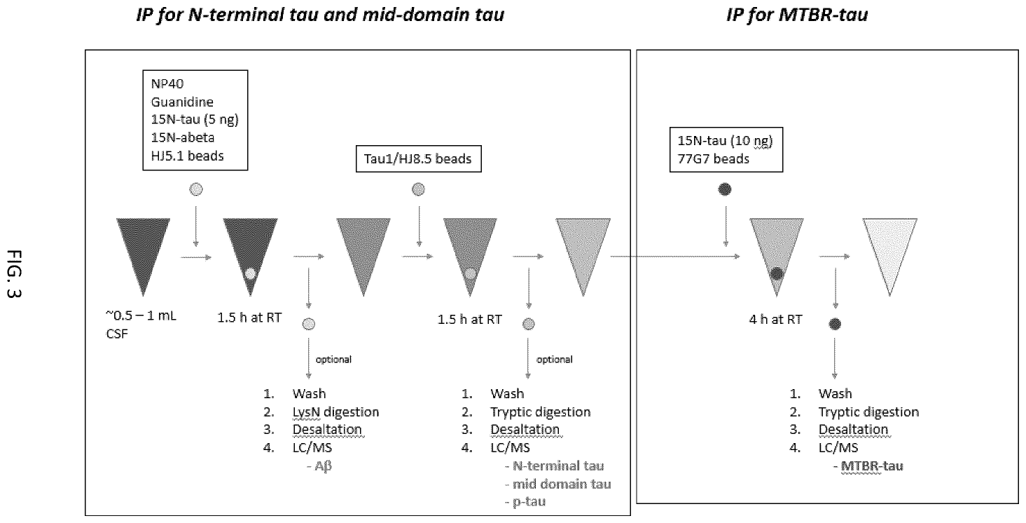

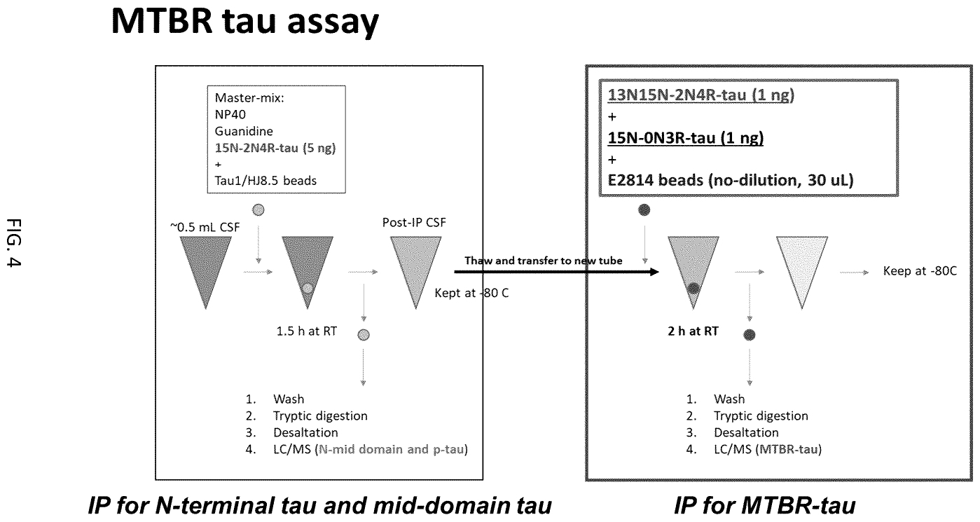

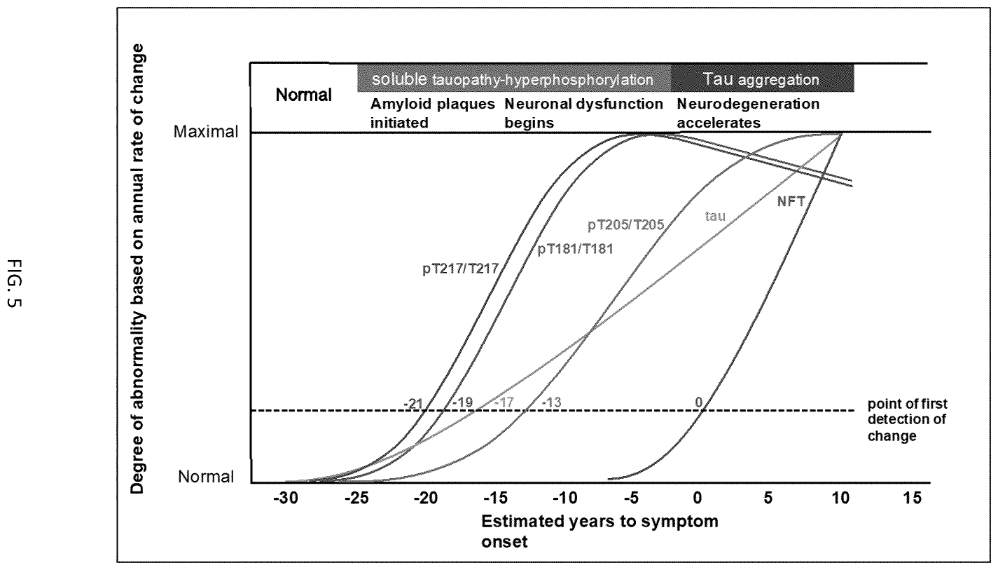

Isotope-labeled tau may be used as an internal standard to account for variability throughout sample processing and optionally to calculate an absolute concentration. Generally, an isotope-labeled, internal tau standard is added before significant sample processing, and it can be added more than once if needed. See, for instance, the methods depicted in FIG. 2-4.

Multiple isotope-labeled internal tau standards are described herein. All have a heavy isotope label incorporated into at least one amino acid residue. One or more full-length isoforms may be used. Alternatively, or in addition, tau isoforms with post-translational modifications and/or peptide fragments of tau may also be used, as is known in the art. Generally speaking, the labeled amino acid residues that are incorporated should increase the mass of the peptide without affecting its chemical properties, and the mass shift resulting from the presence of the isotope labels must be sufficient to allow the mass spectrometry method to distinguish the internal standard (IS) from endogenous tau analyte signals. As shown herein, suitable heavy isotope labels include, but are not limited to ²H, ¹³C, and ¹⁵N. Typically, about 1-10 ng of internal standard is usually sufficient.

### (c) Depleting One or More Protein

Methods of the present disclosure comprise a step wherein one or more protein is depleted from a sample. The term “deplete” means to diminish in quantity or number. Accordingly, a sample depleted of a protein may have any amount of the protein that is measurably less than the amount in the original sample, including no amount of the protein.

Protein(s) may be depleted from a sample by a method that specifically targets one or more protein, for example by affinity depletion, solid phase extraction, or other method known in the art. Targeted depletion of a protein, or multiple proteins, may be used in situations where downstream analysis of that protein is desired (e.g., identification, quantification, analysis of post-translation modifications, etc.). For instance, Aβ peptides may be identified and quantified by methods known in the art following affinity depletion of Aβ with a suitable epitope-binding agent. As another non-limiting example, apolipoprotein E (ApoE) status may be determined by methods known in the art following affinity depletion of ApoE and identification of the ApoE isoform. Targeted depletion may also be used to isolate other proteins for subsequent analysis including, but not limited to, apolipoprotein J, synuclein, soluble amyloid precursor protein, alpha-2 macroglobulin, S100B, myelin basic protein, an interleukin, TNF, TREM-2, TDP-43, YKL-40, VILIP-1, NFL, prion protein, pNFH, and DJ-1. Targeted depletion of certain tau proteins is also used herein to enrich for other tau proteins and/or eliminate proteins that cofound the mass spectrometry analysis. For instance, in certain embodiments of the present disclosure, N-terminal tau proteins and/or mid-domain tau proteins are depleted from a sample prior to further sample processing for analysis by mass spectrometry. Downstream analysis of the depleted tau proteins may or may not occur, but both options are contemplated by the methods of the present disclosure.

In some embodiments, targeted depletion may occur by affinity depletion. Affinity depletion refers to methods that deplete a protein of interest from a sample by virtue of its specific binding properties to a molecule. Typically, the molecule is a ligand attached to a solid support, such as a bead, resin, tissue culture plate, etc. (referred to as an immobilized ligand). Immobilization of a ligand to a solid support may also occur after the ligand-protein interaction occurs. Suitable ligands include antibodies, aptamers, and other epitope-binding agents. The molecule may also be a polymer or other material that selectively absorbs a protein of interest. As a non-limiting example, polyhydroxymethylene substituted by fat oxethylized alcohol (e.g., PHM-L LIPOSORB, Sigma Aldrich) may be used to selectively absorb lipoproteins (including ApoE) from serum. Two or more affinity depletion agents may be combined to sequentially or simultaneously deplete multiple proteins.

In some embodiments, a method of the present disclosure comprises affinity depleting one or more protein from a sample using at least one epitope-binding agent that specifically binds to an epitope within amino acids 1 to 243 of tau-441, inclusive (or within a similarly defined region for 0N or 1N isoforms). In various embodiments, one, two, three or more epitope-binding agents may be used. When two or more epitope-binding agents are used, they may be used sequentially or simultaneously.

In some embodiments, a method of the present disclosure comprises affinity depleting one or more protein from a sample using an epitope-binding agent that specifically binds to an epitope within the N-terminus of tau (e.g., amino acids 1 to 103 of tau-441, inclusive), and an epitope-binding agent that specifically binds to an epitope within the mid-domain of tau (e.g., amino acids 104 to 243 of tau-441, inclusive). The epitope-binding agents may be used sequentially or simultaneously.

In some embodiments, a method of the present disclosure comprises affinity depleting one or more protein from a sample using an epitope-binding agent that specifically binds to an epitope within amino acids 1 to 35 of tau-441, inclusive, and an epitope-binding agent that specifically binds to an epitope within amino acids 104 to 243 of tau-441, inclusive (or within similarly defined regions for 0N or 1N isoforms). The epitope-binding agents may be used sequentially or simultaneously.

In some embodiments, a method of the present disclosure comprises affinity depleting one or more protein from a sample using an epitope-binding agent that specifically binds to an epitope within amino acids 1 to 103 of tau-441, inclusive (or within a similarly defined region for 0N or 1N isoforms); an epitope-binding agent that specifically binds to an epitope within amino acids 104 to 243 of tau-441, inclusive (or within a similarly defined region for 0N or 1N isoforms); and an epitope binding agent that specifically binds to an epitope of amyloid beta. The epitope-binding agents may be used sequentially or simultaneously.

In some embodiments, a method of the present disclosure comprises affinity depleting one or more protein from a sample using an epitope-binding agent that specifically binds to an epitope within amino acids 1 to 35 of tau-441, inclusive (or within a similarly defined region for 0N or 1N isoforms); an epitope-binding agent that specifically binds to an epitope within amino acids 104 to 243 of tau-441, inclusive (or within a similarly defined region for 0N or 1N isoforms); and an epitope binding agent that specifically binds to an epitope of amyloid beta. The epitope-binding agents may be used sequentially or simultaneously.

In some embodiments, a method of the present disclosure comprises affinity depleting one or more protein from a sample using an epitope-binding agent that specifically binds to an epitope within amino acids 1 to 103 of tau-441, inclusive (or within a similarly defined region for 0N or 1N isoforms); and an epitope-binding agent that specifically binds to an epitope of amyloid beta. The epitope-binding agents may be used sequentially or simultaneously.

In some embodiments, a method of the present disclosure comprises affinity depleting one or more protein from a sample using an epitope-binding agent that specifically binds to an epitope within amino acids 1 to 35 of tau-441, inclusive (or within a similarly defined region for 0N or 1N isoforms); and an epitope-binding agent that specifically binds to an epitope of amyloid beta. The epitope-binding agents may be used sequentially or simultaneously.

In some embodiments, a method of the present disclosure comprises affinity depleting one or more protein from a sample using an epitope-binding agent that specifically binds to an epitope within amino acids 104 to 243 of tau-441, inclusive (or within a similarly defined region for 0N or 1N isoforms); and an epitope binding agent that specifically binds to an epitope of amyloid beta. The epitope-binding agents may be used sequentially or simultaneously.

In each of the above embodiments, the epitope binding agent may comprise an antibody or an aptamer. In some embodiments, the epitope-binding agent that specifically binds to amyloid beta is HJ5.1 or is an epitope-binding agent that binds the same epitope as HJ5.1 and/or competitively inhibits HJ5.1. In some embodiments, the epitope-binding agent that specifically binds to that specifically binds to an epitope within amino acids 1 to 103 of tau-441, inclusive, is HJ8.5, or is an epitope-binding agent that binds the same epitope as HJ8.5 and/or competitively inhibits HJ8.5. In some embodiments, the epitope-binding agent that specifically binds to that specifically binds to an epitope within amino acids 104 to 221 of tau-441, inclusive, is Tau1, or is an epitope-binding agent that binds the same epitope as Tau1 and/or competitively inhibits Tau1. Methods for identifying epitopes to which an antibody specifically binds, and assays to evaluate competitive inhibition between two antibodies, are known in the art.

Alternatively, protein(s) may be depleted from a sample by a more general method, for example by ultrafiltration or protein precipitation with an acid, an organic solvent or a salt. Generally speaking, these methods are used to reliably reduce high abundance and high molecular weight proteins, which in turn enriches for low molecular weight and/or low abundance proteins and peptides (e.g., tau, Aβ, etc.).

In some embodiments, proteins may be depleted from a sample by precipitation. Briefly, precipitation comprises adding a precipitating agent to a sample and thoroughly mixing, incubating the sample with precipitating agent to precipitate proteins, and separating the precipitated proteins by centrifugation or filtration. The resulting supernatant may then be used in downstream applications. The amount of the reagent needed may be experimentally determined by methods known in the art. Suitable precipitating agents include perchloric acid, trichloroacetic acid, acetonitrile, methanol, and the like. In an exemplary embodiment, proteins are depleted from a sample by acid precipitation. In a further embodiment, proteins are depleted from a sample by acid precipitation using perchloric acid.

As a non-limiting example, proteins may be depleted from a sample by acid precipitation using perchloric acid. As used herein, “perchloric acid” refers to 70% perchloric acid unless otherwise indicated. In some embodiments, perchloric acid is added to a final concentration of about 1% v/v to about 15% v/v. In other embodiments, perchloric acid is added to a final concentration of about 1% v/v to about 10% v/v. In other embodiments, perchloric acid is added to a final concentration of about 1% v/v to about 5% v/v. In other embodiments, perchloric acid is added to a final concentration of about 3% v/v to about 15% v/v. In other embodiments, perchloric acid is added to a final concentration of about 3% v/v to about 10% v/v. In other embodiments, perchloric acid is added to a final concentration of about 3% v/v to about 5% v/v. In other embodiments, perchloric acid is added to a final concentration of 3.5% v/v to about 15% v/v, 3.5% v/v to about 10% v/v, or 3.5% v/v to about 5% v/v. In other embodiments, perchloric acid is added to a final concentration of about 3.5% v/v. Following addition of the perchloric acid, the sample is mixed well (e.g., by a vortex mixer) and held at a cold temperature, typically for about 10 minutes or longer, to facilitate precipitation. For example, samples may be held for about 10 minutes to about 60 minutes, about 20 minutes to about 60 minutes, or about 30 minutes to about 60 minutes. In other example, samples may be held for about 15 minutes to about 45 minutes, or about 30 minutes to about 45 minutes. In other examples, samples may be held for about 15 minutes to about 30 minutes, or about 20 minutes to about 40 minutes. In other examples, samples are held for about 30 minutes. The sample is then centrifuged at a cold temperature to pellet the precipitated protein, and the supernatant (i.e., the acid soluble fraction), comprising soluble tau, is transferred to a fresh vessel. As used in the above context, a “cold temperature” refers to a temperature of 10° C. or less. For instance, a cold temperature may be about 1° C., about 2° C., about 3° C., about 4° C., about 5° C., about 6° C., about 7° C., about 8° C., about 9° C., or about 10° C. In some embodiments, a narrower temperature range may be preferred, for example, about 3° C. to about 5° C., or even about 4° C. In certain embodiments, a cold temperature may be achieved by placing a sample on ice.

Two or more methods from one or both of the above approaches may be combined to sequentially or simultaneously deplete multiple proteins. For instance, one or more proteins may be selectively depleted (targeted depletion) followed by depletion of high abundance/molecular weight proteins. Alternatively, high abundance/molecular weight proteins may be first depleted followed by targeted depletion of one or more proteins. In still another alternative, high abundance/molecular weight proteins may be first depleted followed by a first round of targeted depletion of one or more proteins and then a second round of targeted depletion of one or more different protein(s) than targeted in the first round. Other iterations will be readily apparent to a skilled artisan.

### (d) Purifying Tau

Another step of the methods disclosed herein comprises purifying tau, in particular MTBR tau. In some examples, the MTBR tau is N-terminal-independent and/or mid-domain-independent MTBR tau. The purified tau may be partially purified or completely purified.

In some embodiments, a method of the present disclosure comprises purifying tau by solid phase extraction. Purifying tau by solid phase extraction comprises contacting a sample comprising tau with a solid phase comprising a sorbent that adsorbs tau, one or more wash steps, and elution of tau from the sorbent. Suitable sorbents include reversed-phase sorbents. Suitable reversed phase sorbents are known in the art and include, but are not limited to alkyl-bonded silicas, aryl-bonded silicas, styrene/divynlbenzene materials, N-vinylpyrrolidone/divynlbenzene materials. In an exemplary embodiment, the reversed phase material is a polymer comprising N-vinylpyrrolidone and divinylbenzene or a polymer comprising styrene and divinylbenzene. In an exemplary embodiment, a sorbent is Oasis HLB (Waters). Prior to contact with the supernatant comprising tau, the sorbent is typically preconditioned per manufacturer's instructions or as is known in the art (e.g., with a water miscible organic solvent and then the buffer comprising the mobile phase). In addition, the supernatant may be optionally acidified, as some reversed-phase materials retain ionized analytes more strongly than others. The use of volatile components in the mobile phases and for elution is preferred, as they facilitate sample drying. In exemplary embodiments, a wash step may comprise the use of a liquid phase comprising about 0.05% v/v trifluoroacetic acid (TFA) to about 1% v/v TFA, or an equivalent thereof. In some examples, the wash may be with a liquid phase comprising about 0.05% v/v to about 0.5% v/v TFA or about 0.05% v/v to about 0.1% v/v TFA. In some examples, the wash may be with a liquid phase comprising about 0.1% v/v to about 1.0% v/v TFA or about 0.1% v/v to about 0.5% v/v TFA. Bound tau is then eluted with a liquid phase comprising about 20% v/v to about 50% v/v acetonitrile (ACN), or an equivalent thereof. In some examples, tau is may be eluted with a liquid phase comprising about 20% v/v to about 40% v/v ACN, or about 20% v/v to about 30% v/v ACN. In some examples, tau is may be eluted with a liquid phase comprising about 30% v/v to about 50% v/v ACN, or about 30% v/v to about 40% v/v ACN. The eluate may be dried by methods known in the art (e.g., vacuum drying (e.g., speed-vac), lyophilization, evaporation under a nitrogen stream, etc.).

In some embodiments, a method of the present disclosure comprises purifying MTBR tau by affinity purification. Affinity purification refers to methods that enrich for a protein of interest by virtue of its specific binding properties to a molecule. Typically, the molecule is a ligand attached to a solid support, such as a bead, resin, tissue culture plate, etc. (referred to as an immobilized ligand). Immobilization of a ligand to a solid support may also occur after the ligand-protein interaction occurs. Suitable ligands include antibodies, aptamers, and other epitope-binding agents. Purifying MTBR tau by affinity purification comprises contacting a sample comprising tau with a suitable immobilized ligand, one or more wash steps, and elution of MTBR tau from the immobilized ligand.

In some embodiments, a method of the present disclosure comprises purifying MTBR tau by affinity purification using at least one epitope-binding agent that specifically binds to an epitope within amino acids 235 to 368 of tau-441, inclusive, or within amino acids 244 to 368 of tau-441, inclusive (or within similarly defined regions for other full-length isoforms). In various embodiments, one, two, three or more epitope-binding agents may be used. When two or more epitope-binding agents are used, they may be used sequentially or simultaneously. Non-limiting examples of suitable epitope-binding agents include antibodies 77G7, RD3, RD4, UCB1017, and PT76 described in Vandermeeren et al., J Alzheimers Dis, 2018, 65:265-281, and antibodies E2814 and 7G6 described in Roberts et al., Acta Neuropathol Commun, 2020, 8: 13, as well as other epitope-binding agents that specifically bind the same epitopes as those antibodies. In further embodiments, a method of the present disclosure comprises purifying MTBR tau by affinity purification using an epitope-binding agent that specifically binds to an epitope within R1 of MTBR tau, an epitope-binding agent that specifically binds to an epitope within R2 of MTBR tau, an epitope-binding agent that specifically binds to an epitope within R3 of MTBR tau, an epitope-binding agent that specifically binds to an epitope within R4 of MTBR tau, an epitope-binding agent that specifically binds to an epitope unique to 3R tau, an epitope-binding agent that specifically binds to an epitope unique to 4R tau, an epitope-binding agent that specifically binds to an epitope spanning R1 and R2 of MTBR tau, an epitope-binding agent that specifically binds to an epitope spanning R2 and R3 of MTBR tau, an epitope-binding agent that specifically binds to an epitope spanning R3 and R4 of MTBR tau, or any combination thereof. In a specific example, a method of the present disclosure comprises purifying MTBR tau by affinity purification using an epitope-binding agent that specifically binds to an epitope comprising amino acids 316 to 355 of tau-441 (or the same region for the other full length isoforms). In various embodiments, one, two, three or more epitope-binding agents may be used. When two or more epitope-binding agents are used, they may be used sequentially or simultaneously.

In each of the above embodiments, the epitope-binding agent may comprise an antibody or an aptamer. In some embodiments, an epitope-binding agent that specifically binds to an epitope within R3 and R4 of MTBR tau is 77G7 or is an epitope-binding agent that binds the same epitope as 77G7 and/or competitively inhibits 77G7 (BioLegend). In some embodiments, an epitope-binding agent that specifically binds to an epitope unique to 3R tau is RD3 (de Silva et al., Neuropathology and Applied Neurobiology, 2003, 29: 288-302), or is an epitope-binding agent that binds the same epitope as RD3 and/or competitively inhibits RD3. In some embodiments, an epitope-binding agent that specifically binds to an epitope unique to 4R tau is RD4 (de Silva et al., Neuropathology and Applied Neurobiology, 2003, 29: 288-302), or is an epitope-binding agent that binds the same epitope as RD4 and/or competitively inhibits RD4.

(e) Cleaving Purified Tau with a Protease

Another step of the methods disclosed herein comprises cleaving purified tau with a protease. Cleaving purified tau with a protease comprises contacting a sample comprising purified tau with a protease under conditions suitable to digest tau. When affinity purification is used, digestion may occur after eluting tau from the immobilized ligand or while tau is bound. Suitable proteases include but are not limited to trypsin, Lys-N, Lys-C, and Arg-N. In a preferred embodiment, the protease is trypsin. The resultant cleavage product is a composition comprising proteolytic peptides of tau. When the protease is trypsin, the resultant cleavage product comprises tryptic peptides of tau. Following proteolytic cleavage, the resultant cleavage product is typically desalted by solid phase extraction.

### (f) LC-MS

Another step of the methods disclosed herein comprises performing liquid chromatography—mass spectrometry (LC-MS) with a sample comprising proteolytic peptides of tau to detect and measure the concentration of at least one proteolytic peptide of tau. Thus, in practice, the disclosed methods use one or more proteolytic peptide of tau to detect and measure the amount of tau protein present in the biological sample.

In embodiments where trypsin is the protease, proteolytic peptides of tau that indicate the presence of MTBR tau include but are not limited to the peptides listed in Table A. When using an alternative enzyme for digestion, the resulting proteolytic peptides may differ slightly but can be readily determined by a person of ordinary skill in the art. Without wishing to be bound by theory, it is believed that a variation in the amount of a tryptic peptide between two biological samples of the same type reflects a difference in the MTBR tau species that make up those biological samples. As disclosed herein, the amounts of certain proteolytic peptides of MTBR tau, as well ratios of certain proteolytic peptides of MTBR tau, may provide clinically meaningful information to guide treatment decisions. Thus, methods that allow for detection and quantification of tryptic peptides of MTBR tau have utility in the diagnosis and treatment of many neurodegenerative diseases.

TABLE A
Tryptic peptides of tau that indicate the
presence of MTBR tau

| Tryptic | | SEQ |
|---|---|---|
| peptide name(s) | Amino acid sequence | ID NO: |
| IGST | IGSTENLK | 2 |
| LQTA | LQTAPVPMPDLK | 3 |
| VQII | VQIINK | 4 |
| LDLS | LDLSNVQSK | 5 |
| HVPG | HVPGGGSVQIVYKPVDLSK | 6 |
| IGSL | IGSLDNITHVPGGGNK | 7 |
| tau368 | IGSLDNITHVPGGGN | 8 |
| VQIV | VQIVYKPVDLSK | 9 |

Proteolytic peptides of tau may be separated by a liquid chromatography system interfaced with a high-resolution mass spectrometer. Suitable LC-MS systems may comprise a <1.0 mm ID column and use a flow rate less than about 100 μl/min. In preferred embodiments, a nanoflow LC-MS system is used (e.g., about 50-100 μm ID column and a flow rate of <1 μL/min, preferably about 100-800 nL/min, more preferably about 200-600 nL/min). In an exemplary embodiment, an LC-MS system may comprise a 0.05 mM ID column and use a flow rate of about 400 nL/min.

Tandem mass spectrometry may be used to improve resolution, as is known in the art, or technology may improve to achieve the resolution of tandem mass spectrometry with a single mass analyzer. Suitable types of mass spectrometers are known in the art. These include, but are not limited to, quadrupole, time-of-flight, ion trap and Orbitrap, as well as hybrid mass spectrometers that combine different types of mass analyzers into one architecture (e.g., Orbitrap Fusion™ Tribrid™ Mass Spectrometer, Orbitrap Fusion™ Lumos™ Mass Spectrometer, Orbitrap Tribrid™ Eclipse™ Mass Spectrometer, Q Exactive Mass Spectrometer, each from ThermoFisher Scientific). In an exemplary embodiment, an LC-MS system may comprise a mass spectrometer selected from Orbitrap Fusion™ Tribrid™ Mass Spectrometer, Orbitrap Fusion™ Lumos™ Mass Spectrometer, Orbitrap Tribrid™ Eclipse™ Mass Spectrometer, or a mass spectrometer with similar or improved ion-focusing and ion-transparency at the quadrupole. Suitable mass spectrometry protocols may be developed by optimizing the number of ions collected prior to analysis (e.g., AGC setting using an orbitrap) and/or injection time. In an exemplary embodiment, a mass spectrometry protocol outlined in the Examples is used.

III. Staging a subject

In an aspect, the present disclosure provides a method for measuring time to dementia onset in a subject without cognitive or behavioral symptoms of Alzheimer's disease, the method comprising (ai) measuring phosphorylation occupancy at residue T205 of tau and measuring MTBR-tau299/MTBR-tau354 ratio, and optionally measuring MTBR-tau212, phosphorylation occupancy at residue T217 MTBR-tau243, and/or MTBR-tau3R, in a blood sample or a CSF sample obtained from the subject, or (aᵢᵢ) measuring phosphorylation occupancy at residue T205 of tau, measuring phosphorylation occupancy at residue T217 of tau, and measuring MTBR-tau212, in a blood sample or a CSF sample obtained from the subject; and (b) using the measurements of (aᵢ) or (aᵢᵢ) to calculate time to dementia onset, wherein time to dementia onset is time in years to a Clinical Dementia Rating (CDR) greater than zero. In an exemplary embodiment, a subject without cognitive or behavioral symptoms of Alzheimer's disease has a CDR of zero.

In another aspect, the present disclosure provides a method for measuring time to dementia onset in a subject without cognitive or behavioral symptoms of Alzheimer's disease, the method comprising (a) processing a blood sample or a CSF sample from the subject to obtain a first population of tau species and a depleted sample, and then processing the depleted sample to obtain a second population of tau species, wherein the first population of tau species is enriched for N-terminal tau and/or mid-domain tau, and wherein the second population of enriched tau species is enriched for MTBR-tau; (bᵢ) measuring phosphorylation occupancy at residue T205 of tau in the first population of tau species and measuring MTBR-tau299/MTBR-tau354 ratio in the second population of tau species, and optionally measuring MTBR-tau212 in the second population of tau species, or (bᵢᵢ) measuring phosphorylation occupancy at residue T205 of tau and measuring phosphorylation occupancy at residue T217 of tau in the first population of tau species, and measuring MTBR-tau212 in the second population of tau species; and (c) calculating time to dementia onset using the measurements of (bᵢ) or (bᵢᵢ), wherein time to dementia onset is time in years to a Clinical Dementia Rating greater than zero. In an exemplary embodiment, a subject with cognitive or behavioral symptoms of Alzheimer's disease has a CDR greater than zero, greater than or equal to 0.5, or greater than or equal to 1. As non-limiting example, a subject with cognitive or behavioral symptoms of Alzheimer's disease may have a CDR of 0.5, 1, 1.5, or 2.

In another aspect, the present disclosure provides a method for measuring time from dementia onset in a subject with cognitive or behavioral symptoms of Alzheimer's disease, the method comprising (aᵢ) measuring phosphorylation occupancy at residue T205 of tau and measuring MTBR-tau299/MTBR-tau354 ratio, and optionally measuring MTBR-tau212, phosphorylation occupancy at residue T217 MTBR-tau243, and/or MTBR-tau3R, in a blood sample or a CSF sample obtained from the subject, or (aᵢᵢ) measuring phosphorylation occupancy at residue T205 of tau, measuring phosphorylation occupancy at residue T217 of tau, and measuring MTBR-tau212, in a blood sample or a CSF sample obtained from the subject; and (b) using the measurements of (aᵢ) or (aᵢᵢ) to calculate time from dementia onset, wherein time from dementia onset is time in years from a Clinical Dementia Rating greater than zero.

In another aspect, the present disclosure provides, a method for measuring time from dementia onset in a subject with cognitive or behavioral symptoms of Alzheimer's disease, the method comprising (a) processing a blood sample or a CSF sample from the subject to obtain a first population of tau species and a depleted sample, and then processing the depleted sample to obtain a second population of tau species, wherein the first population of tau species is enriched for N-terminal tau and/or mid-domain tau, and wherein the second population of enriched tau species is enriched for MTBR-tau; (bᵢ) measuring phosphorylation occupancy at residue T205 of tau in the first population of tau species and measuring MTBR-tau299/MTBR-tau354 ratio in the second population of tau species, and optionally measuring MTBR-tau212 in the second population of tau species, or (bᵢᵢ) measuring phosphorylation occupancy at residue T205 of tau and measuring phosphorylation occupancy at residue T217 of tau in the first population of tau species, and measuring MTBR-tau212 in the second population of tau species; and (c) calculating time from dementia onset using the measurements of (bᵢ) or (bᵢᵢ), wherein time from dementia onset is time in years from a Clinical Dementia Rating greater than zero.

In another aspect, the present disclosure provides a method for measuring time from dementia onset in a subject with cognitive or behavioral symptoms of Alzheimer's disease, the method comprising (aᵢ) measuring phosphorylation occupancy at residue T205 of tau and measuring the rate of change of MTBR-tau (e.g., MTBR-tau299), and optionally measuring MTBR-tau212, in a blood sample or a CSF sample obtained from the subject; and (b) using the measurements of (aᵢ) or (aᵢᵢ) to calculate time from dementia onset, wherein time from dementia onset is time in years from a Clinical Dementia Rating greater than zero.

In another aspect, the present disclosure provides a method for measuring change in cognition in a subject, the method comprising (a) measuring phosphorylation occupancy at one or more residue of tau selected from T111, T153, T181, T217 and T231 in a blood sample or a CSF sample obtained from the subject, and measuring at least one of MTBR-tau275, MTBR-tau299, and MTBR-tau3R (MTBR-tau306) in a blood sample or a CSF sample obtained from the subject, and optionally measuring total tau in a blood sample or a CSF sample obtained from the subject; and (b) using the measurements of (a) to calculate a change in cognition.

In some of the above embodiments, calculating time to dementia onset includes determining the amount the measured tau species level(s) significantly deviate from the mean in a control population without brain amyloid plaques as measured by PET imaging and/or Aβ42/40 measurements in CSF. “Significantly deviate from the mean” refers to values that are at least 1 standard deviation, preferably at least 1.3 standard deviations, more preferably at least 1.5 standard deviations or even more preferably at least 2 standard deviations, above or below the mean (i.e., 1σ, 1.3σ, 1.5σ, or 1.5σ, respectively, where σ is the standard deviation defined by the normal distribution measured in a control population without brain amyloid plaques as measured by PET imaging and/or Aβ42/40 measurement in CSF). In addition to using a threshold (e.g. at least 1 standard deviation above or below the mean), in some embodiment the extent of change above or below the mean, or the rate of change over time, may be used to calculate time to dementia onset in a subject.

A biological sample can be obtained from a subject that may or may not be asymptomatic. An “asymptomatic subject” refers to a subject that does not show any signs or symptoms of AD. A subject may however exhibit signs or symptoms of AD (e.g., memory loss, misplacing things, changes in mood or behavior, etc.,) but not show sufficient cognitive or functional impairment for a clinical diagnosis of mild cognitive impairment or dementia. In further embodiments, a subject may carry one of the gene mutations known to cause dominantly inherited Alzheimer's disease. In alternative embodiments, a subject may not carry a gene mutation known to cause dominantly inherited Alzheimer's disease. Alzheimer's disease that has no specific family link is referred to as sporadic Alzheimer's disease.

Another aspect of the present disclosure encompasses methods to diagnose a subject's stage of Alzheimer's disease. In various embodiments, a “stage of AD” may be defined as an amount of time (e.g. 1, 2, 3, 4, 5, 6, 7, 8, 9, 10, 11, 12 months, etc.) that has elapsed since the onset of dementia due to AD. Although there are criteria for a clinical diagnosis of AD, it is common in the clinical setting for the timing of symptom onset to be unknown for a given subject or for there to be a questionable diagnosis of either dementia, dementia, or AD. As such, there is a need in the art for a test that objectively diagnoses a subject's stage of AD.

Alternatively or in addition to using a measurement of site-specific tau phosphorylation, MTBR-tau species, and optionally a measurement of total tau, in any of the above embodiments, a ratio calculated from the measured phosphorylation level(s) and/or MTBR-tau levels, or a ratio calculated from the measured phosphorylation level(s) and/or MTBR-tau levels and total tau, may be used. Both approaches are detailed in the examples. Mathematical operations other than a ratio may also be used. For instance, the examples use site-specific tau phosphorylation values and/or MTBR-tau values in various statistical models (e.g., linear regressions, LME curves, LOESS curves, etc.) in conjunction with other known biomarkers (e.g. APOE E4 status, age, sex, cognitive test scores, functional test scores, etc.). Selection of measurements and choice of mathematical operations may be optimized to maximize specificity of the method. For instance, diagnostic accuracy may be evaluated by area under the ROC curve and in some embodiments, an ROC AUC value of 0.7 or greater is set as a threshold (e.g., 0.7, 0.75, 0.8, 0.85, 0.9, 0.95, etc.).

Brain amyloid plaques in humans are routinely measured by amyloid-positron emission tomography (PET). For instance, ¹¹C-Pittsburgh compound B (PiB) PET imaging of cortical Aβ-plaques is commonly used to detect Aβ-plaque pathology. The standard uptake value ratio (SUVR) of cortical PiB-PET reliably identifies significant cortical Aβ-plaques and is used to classify subjects as PIB positive (SUVR>1.25) or negative (SUVR<1.25). Accordingly, in the above embodiments, a control population without brain amyloid plaques as measured by PET imaging may refer to a population of subjects that have a cortical PiB-PET SUVR<1.25. Other values of PiB binding (e.g., mean cortical binding potential) or analyses of regions of interest other than the cortical region may also be used to classify subjects as PIB positive or negative. Other PET imaging agents may also be used.

A control population without brain amyloid plaques as measured by Aβ42/40 measurement in CSF may refer to a population of subjects that has an Aβ42/40 measurement of <0.12 when measured by mass spectrometry, as described in Patterson et al, Annals of Neurology, 2015.

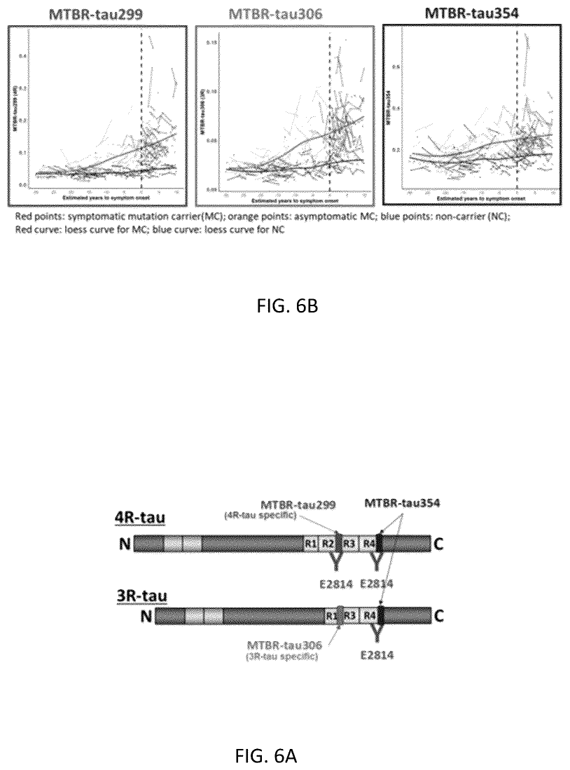

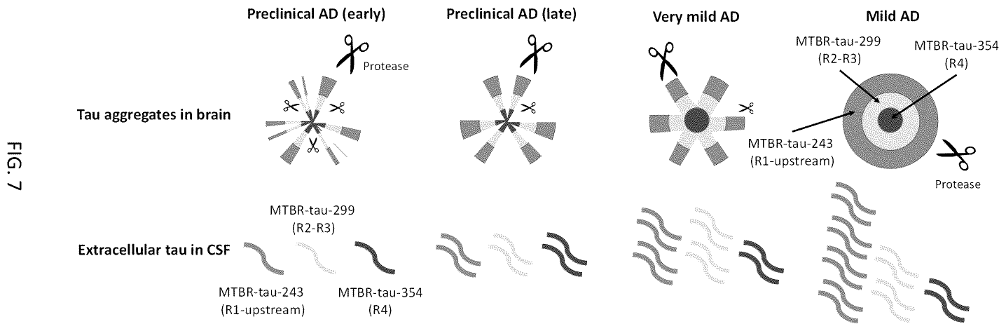

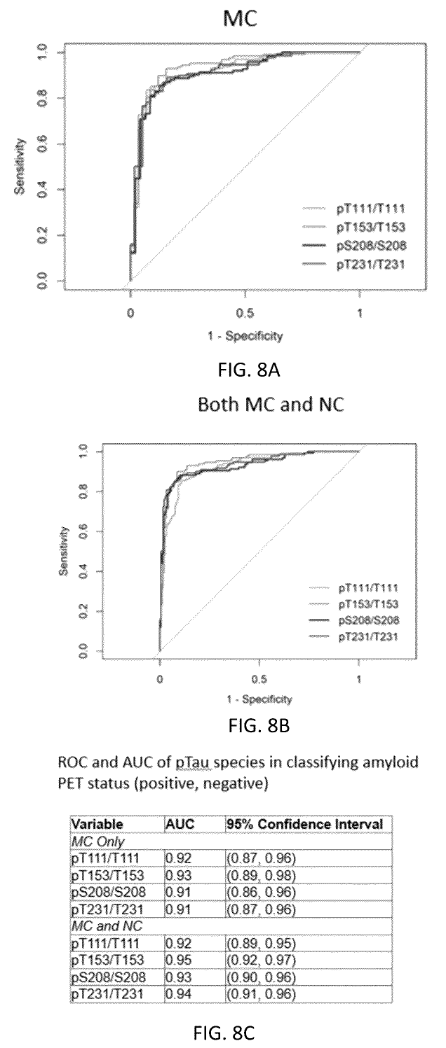

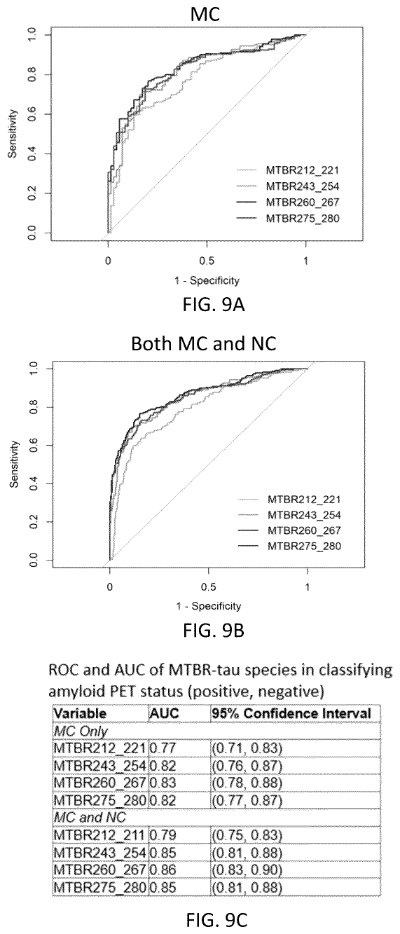

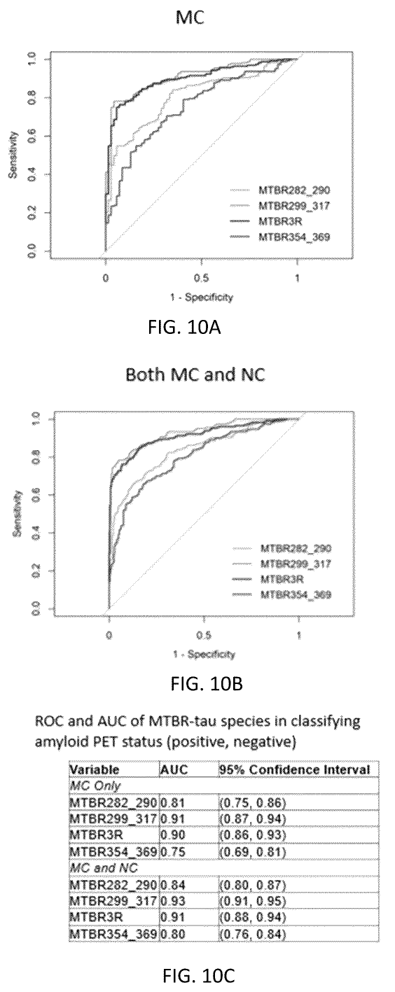

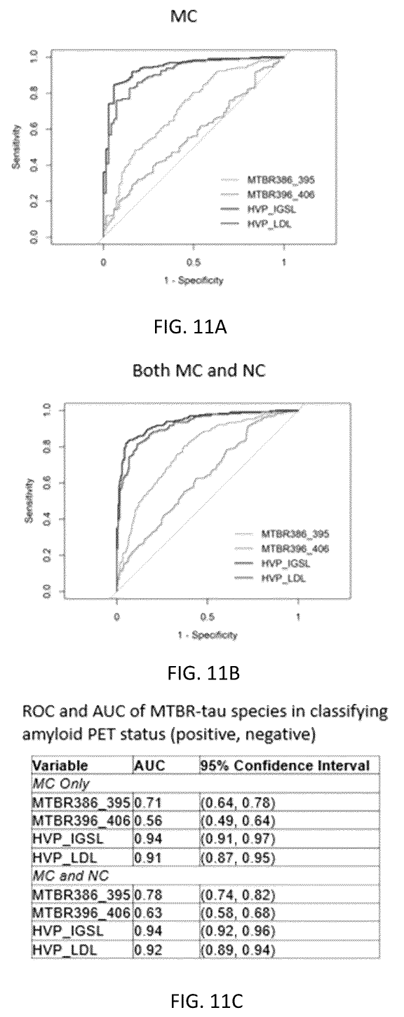

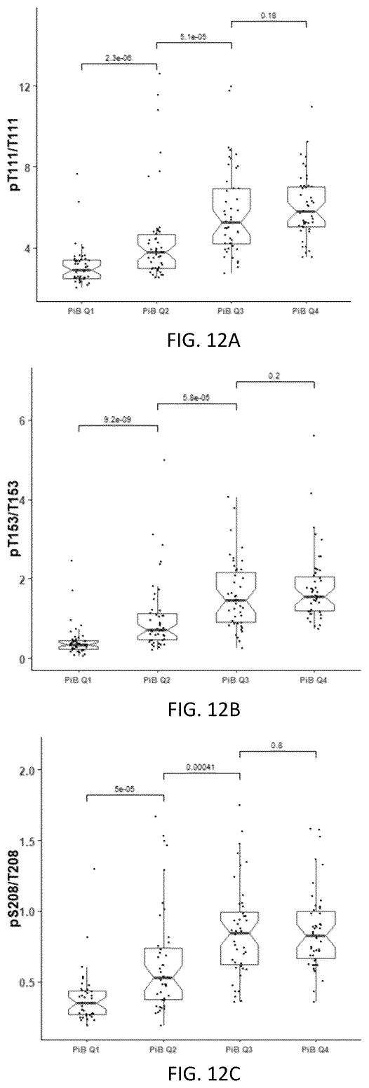

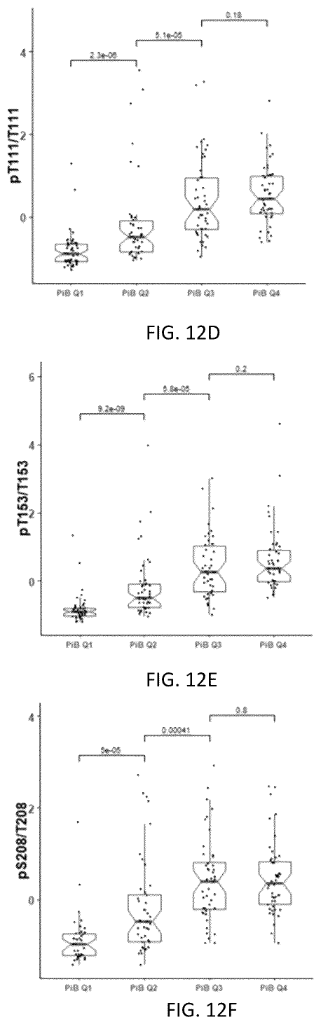

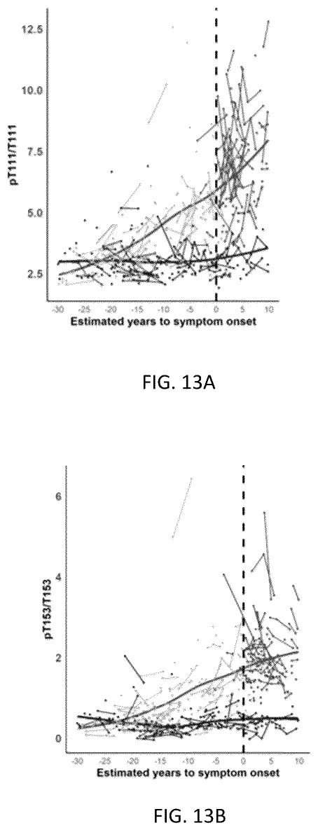

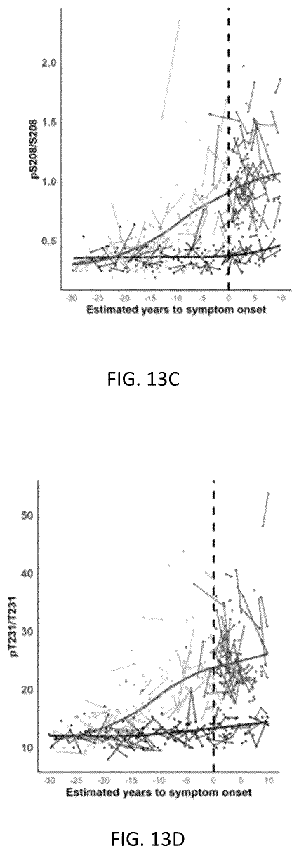

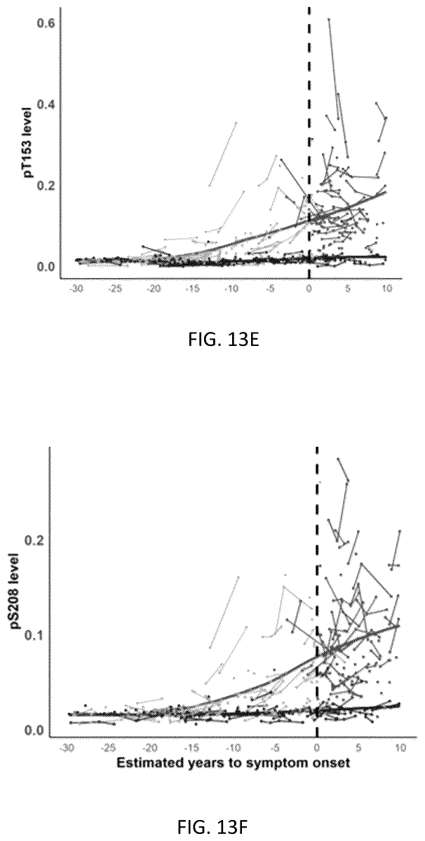

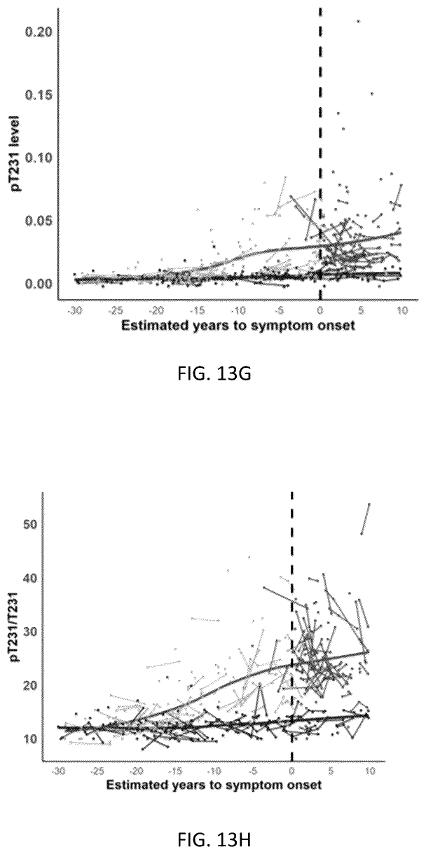

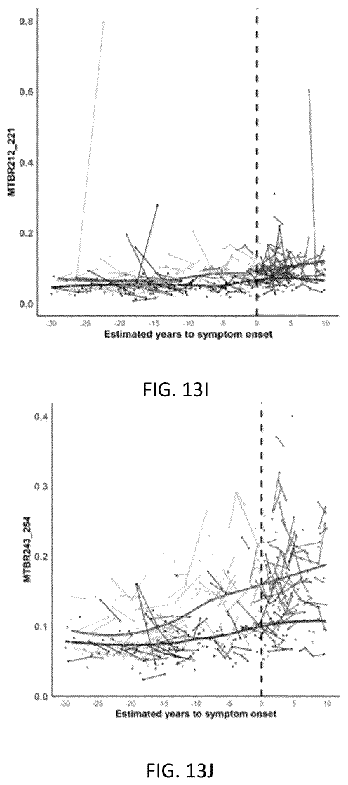

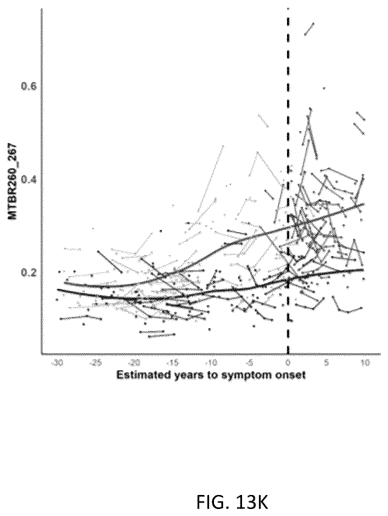

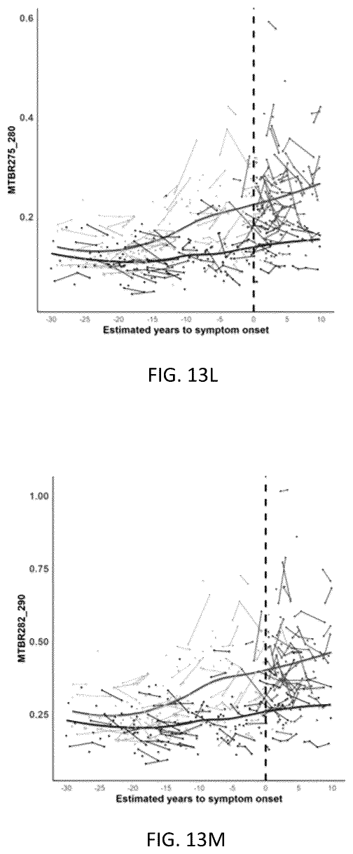

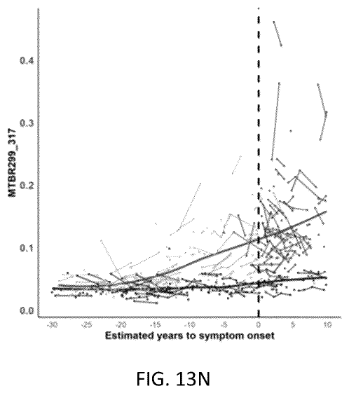

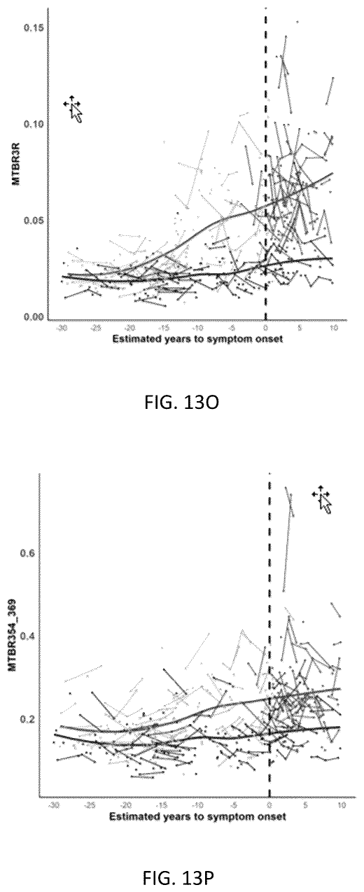

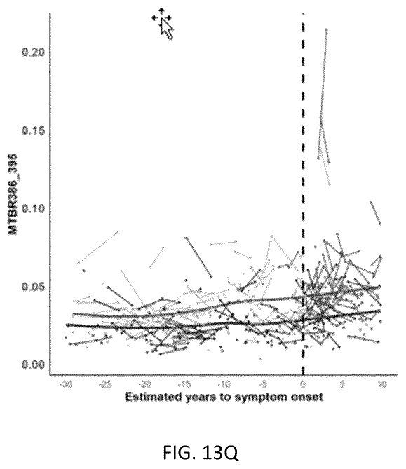

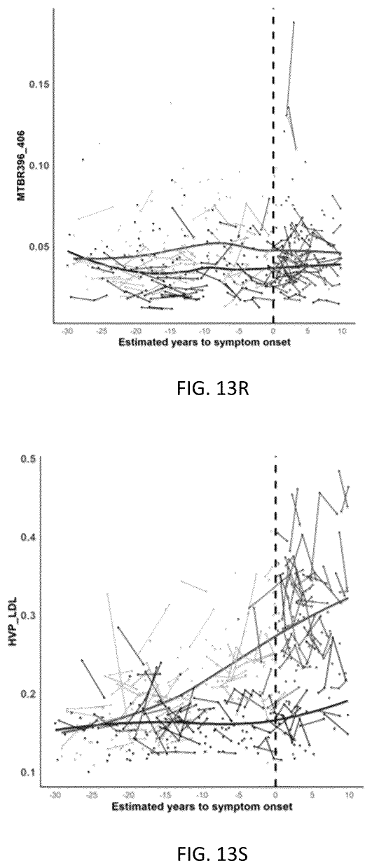

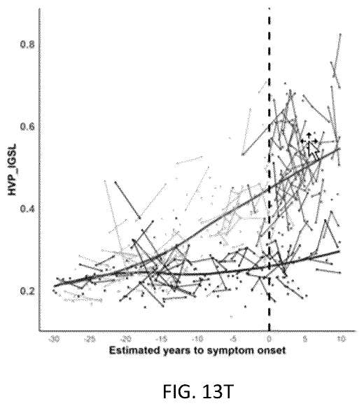

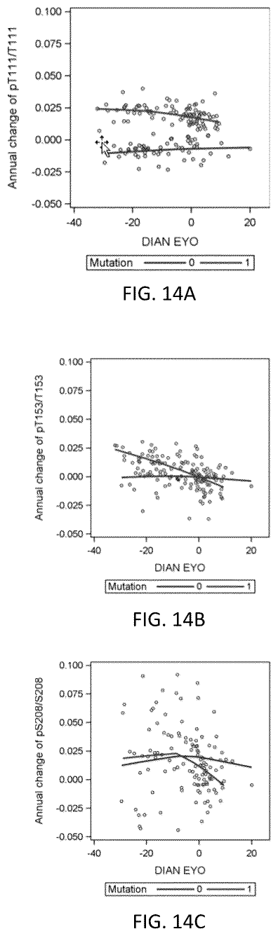

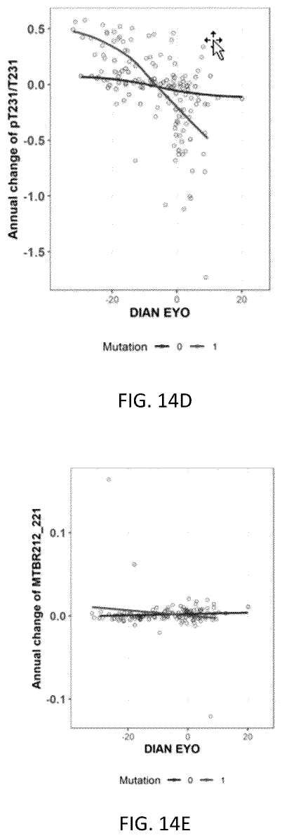

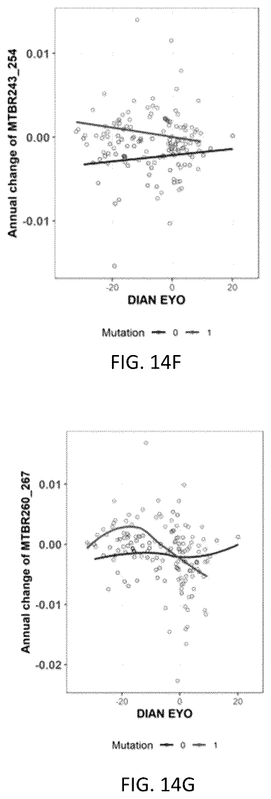

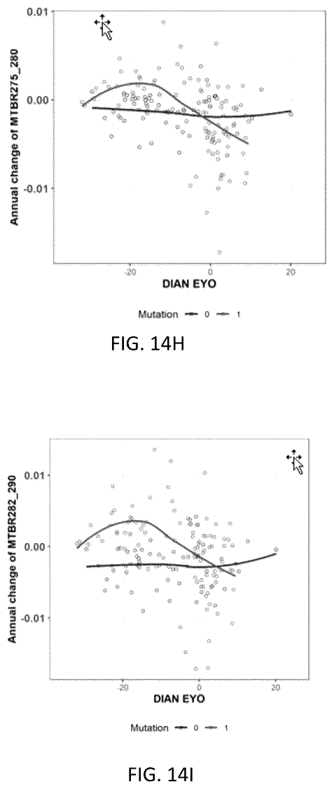

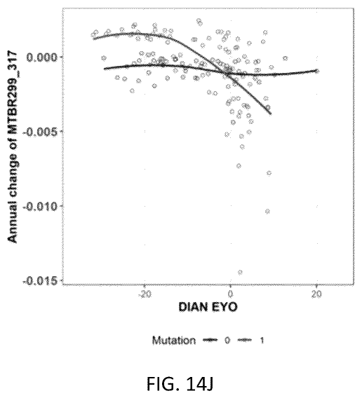

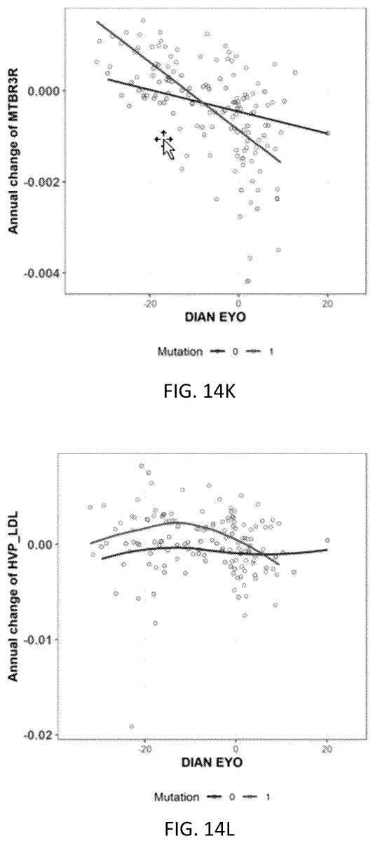

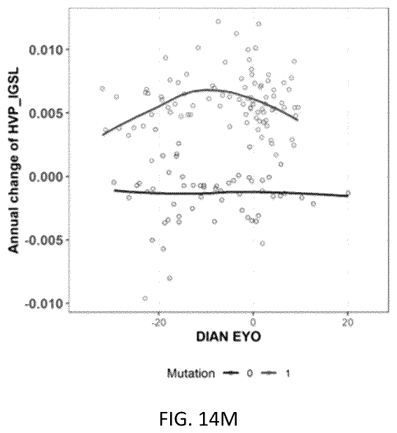

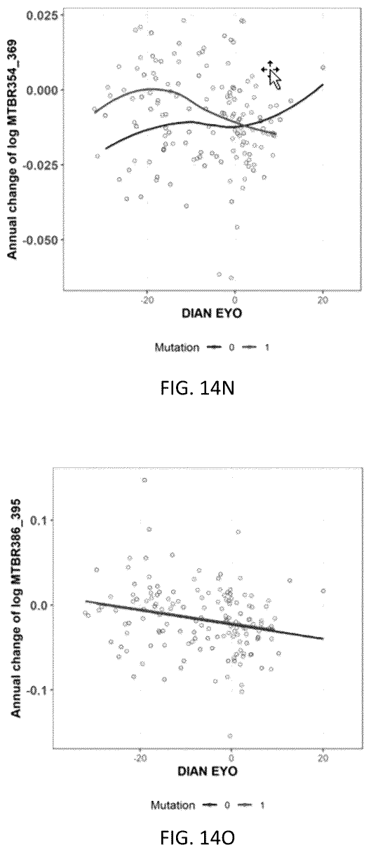

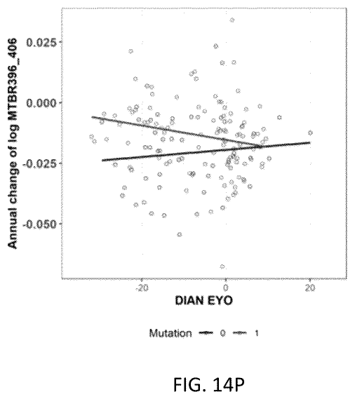

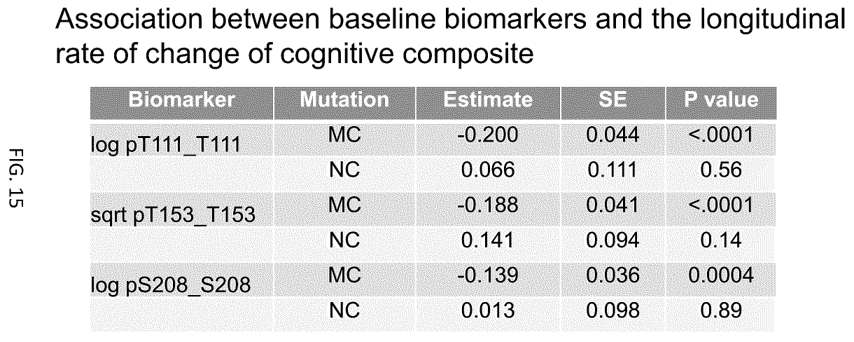

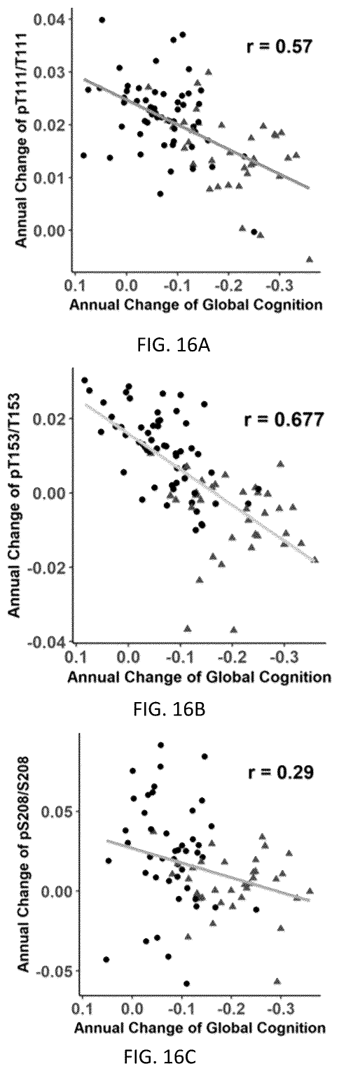

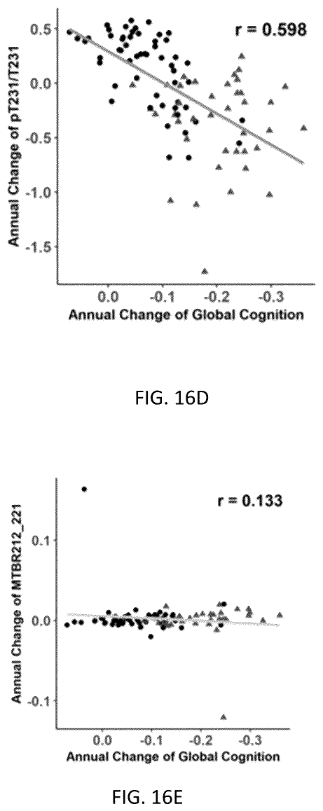

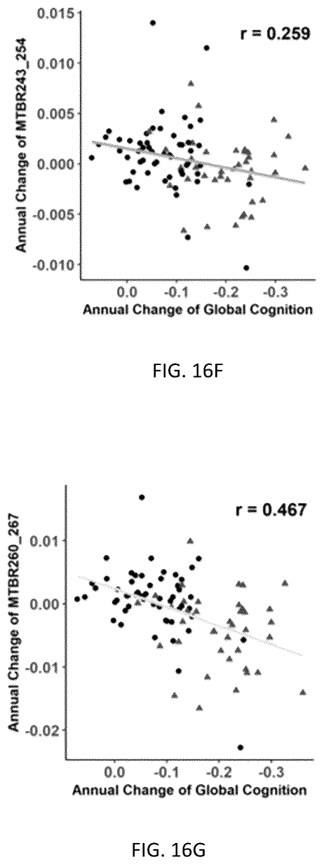

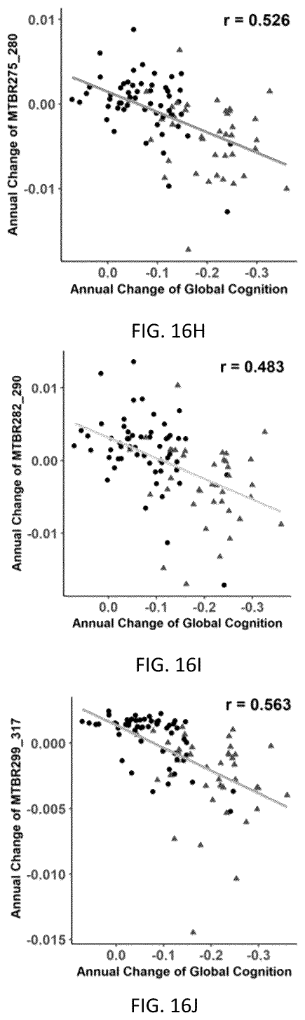

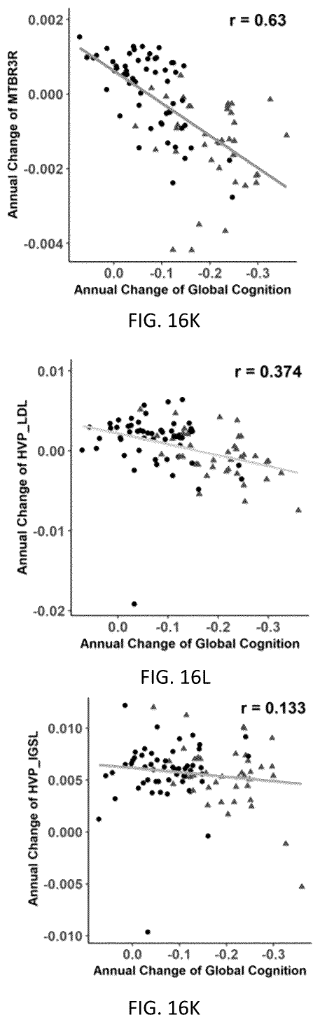

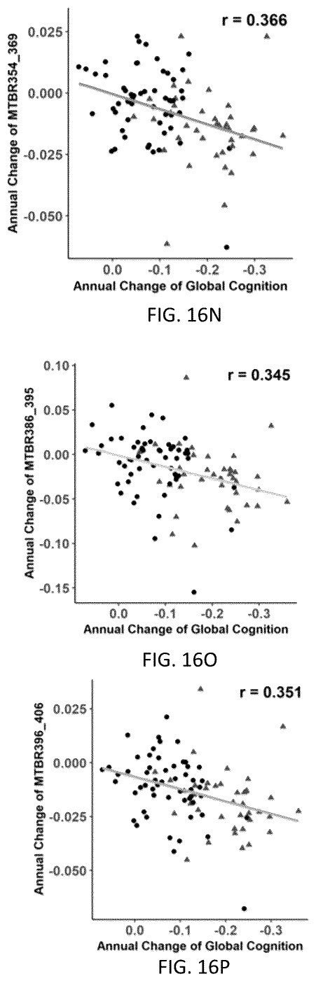

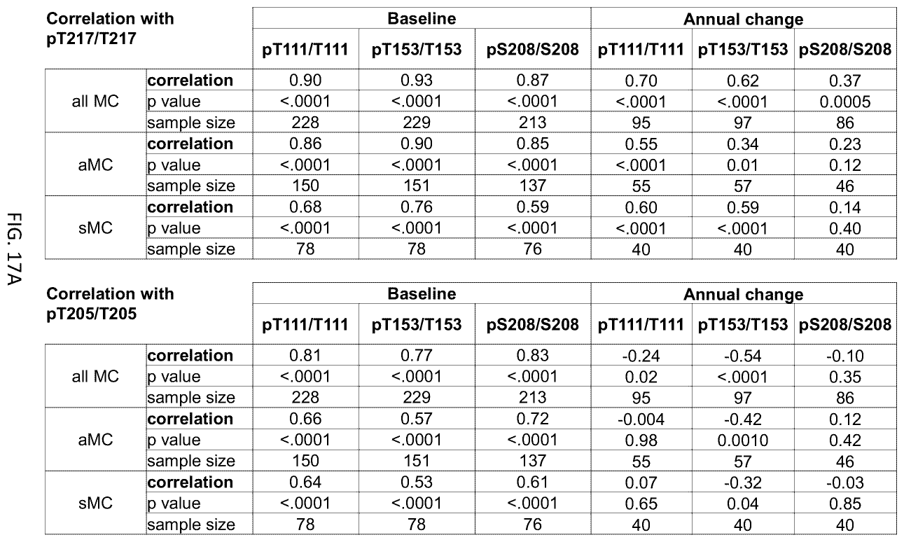

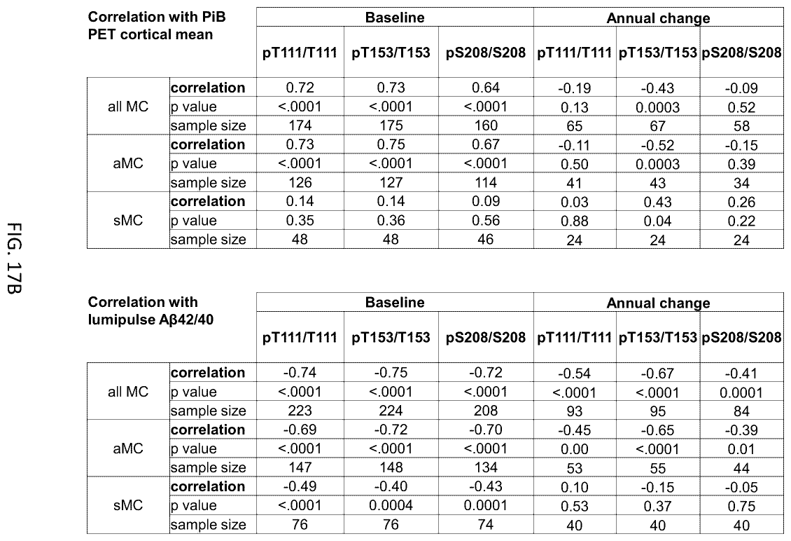

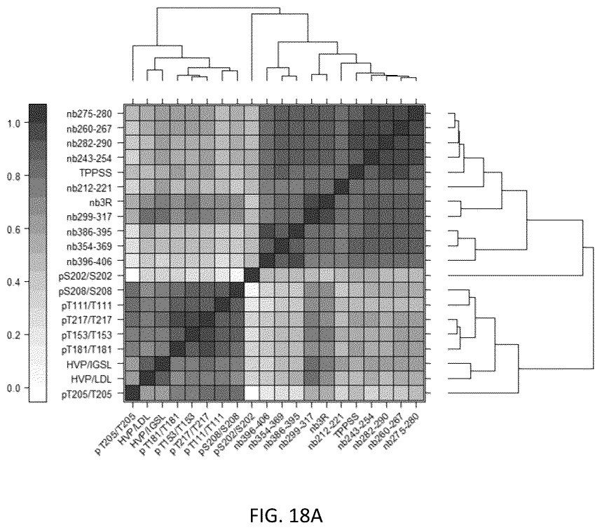

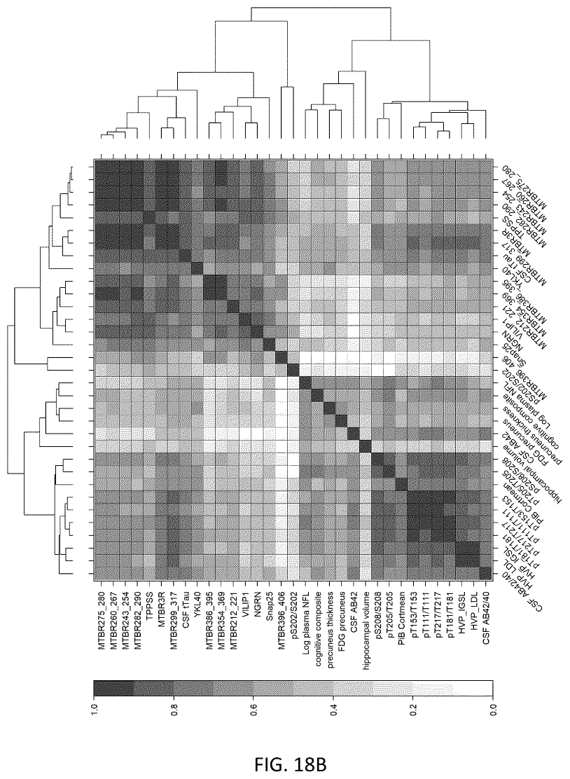

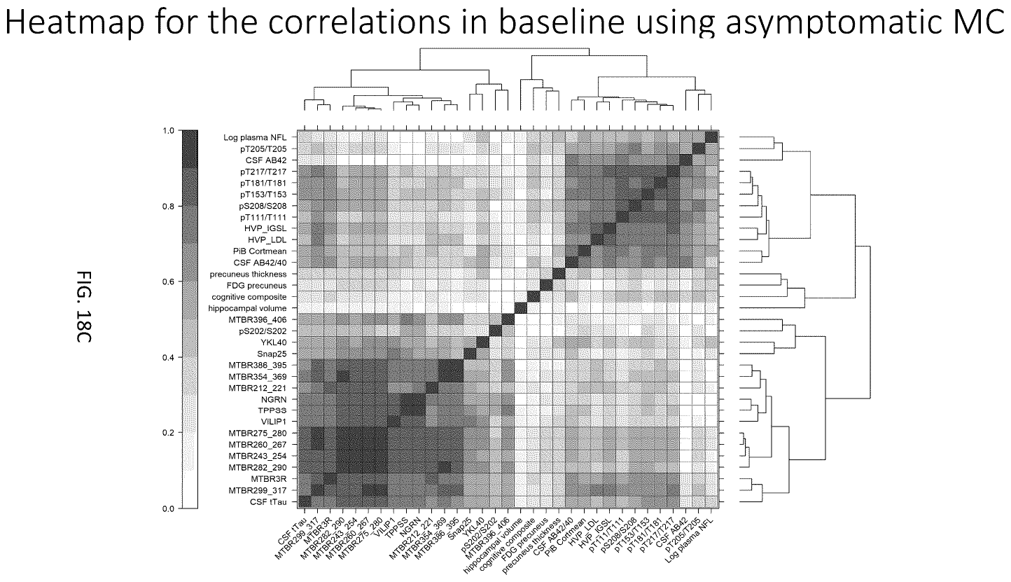

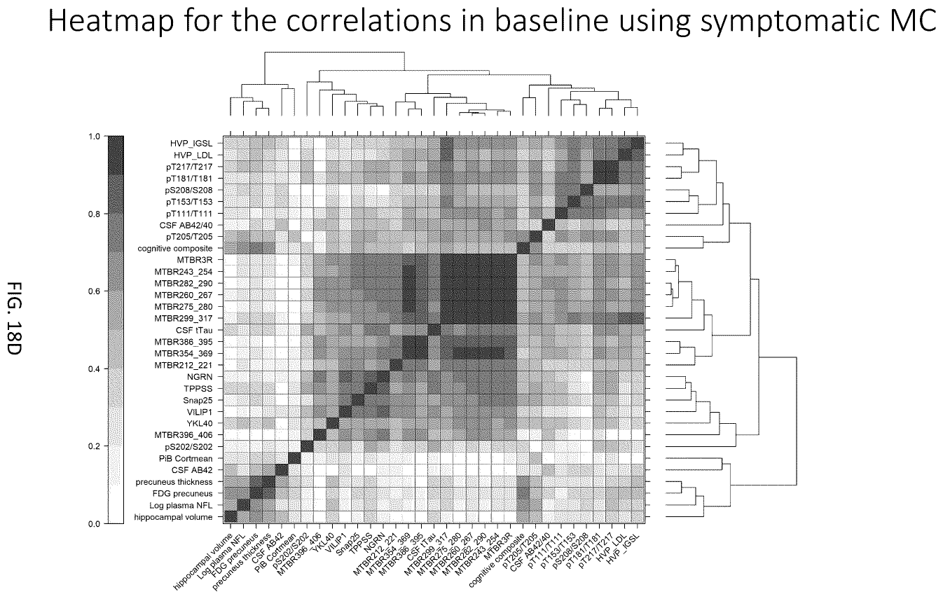

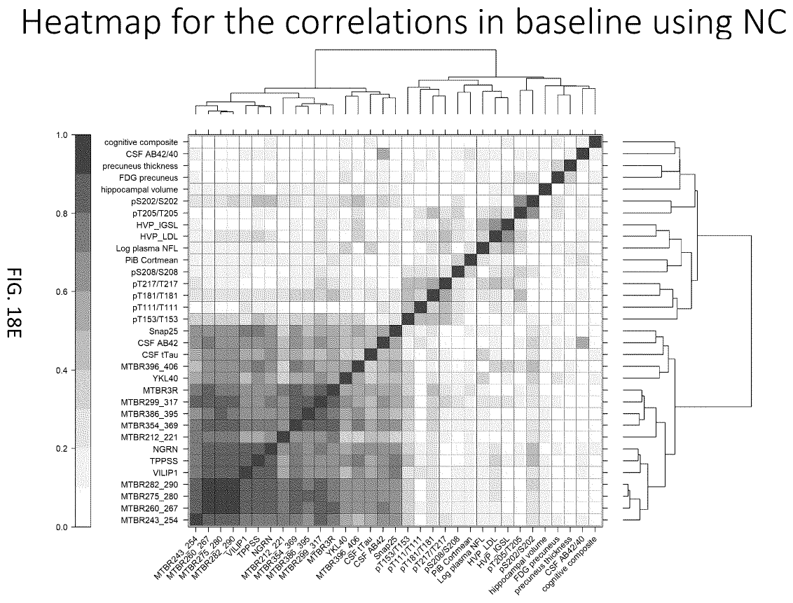

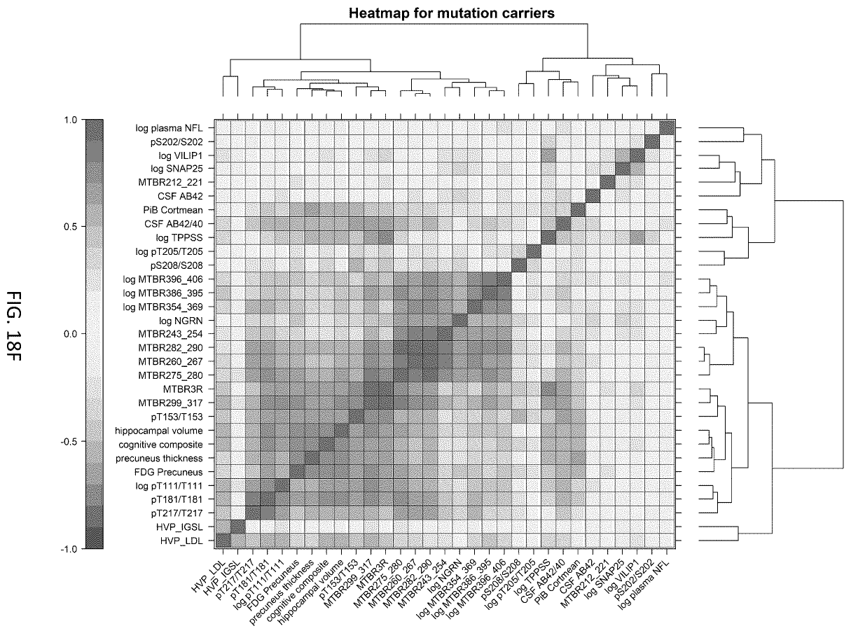

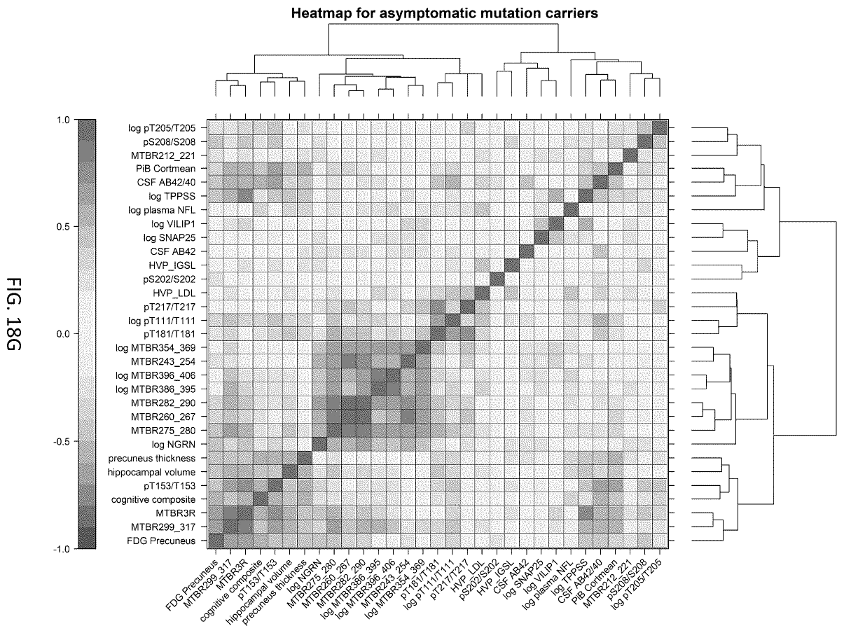

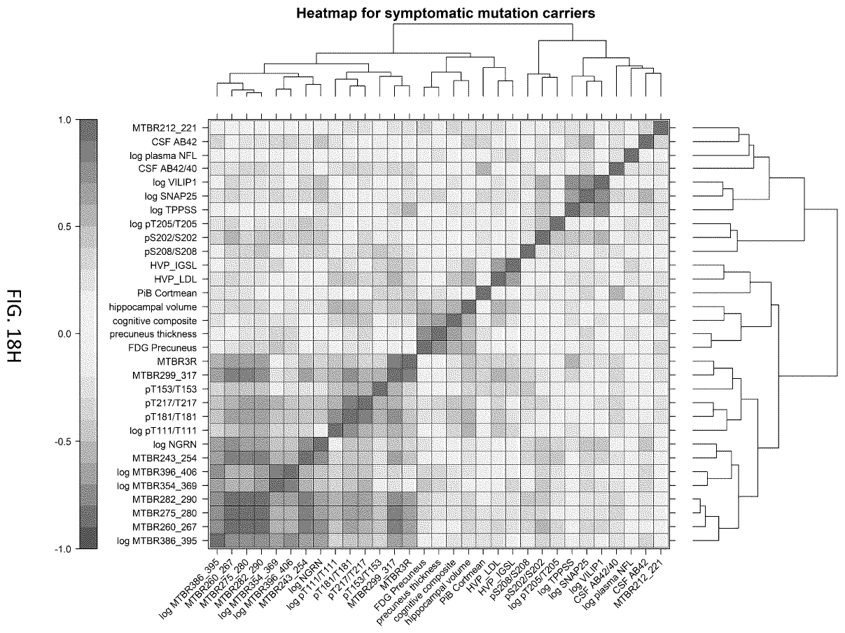

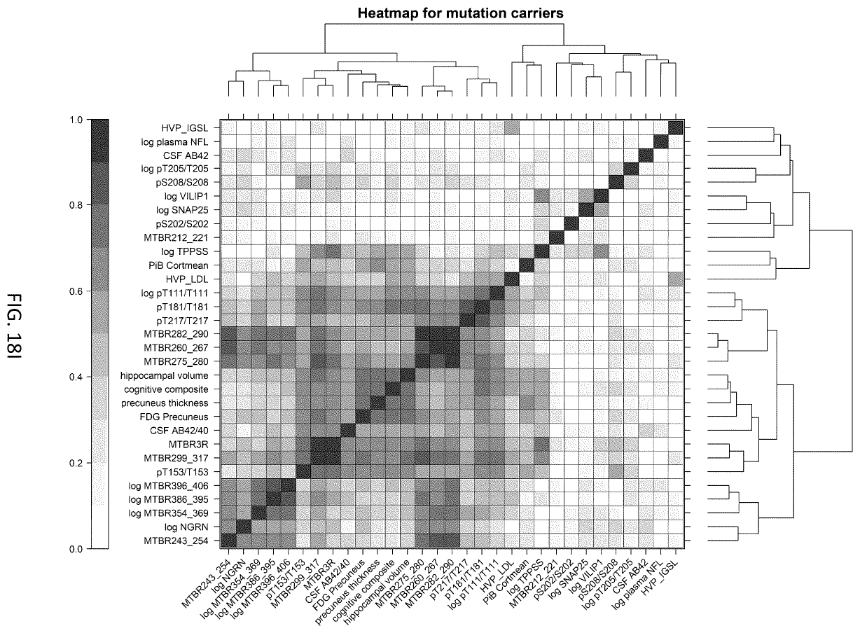

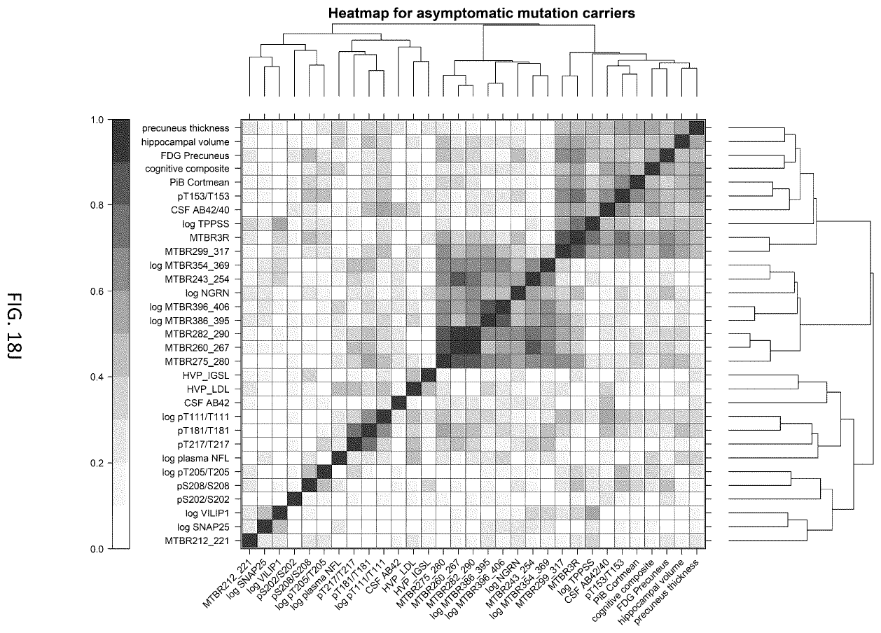

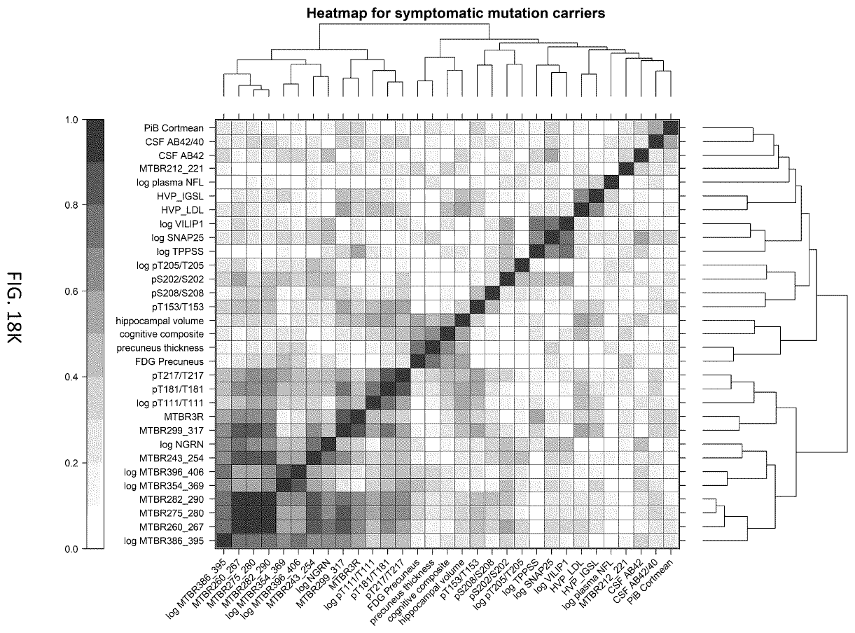

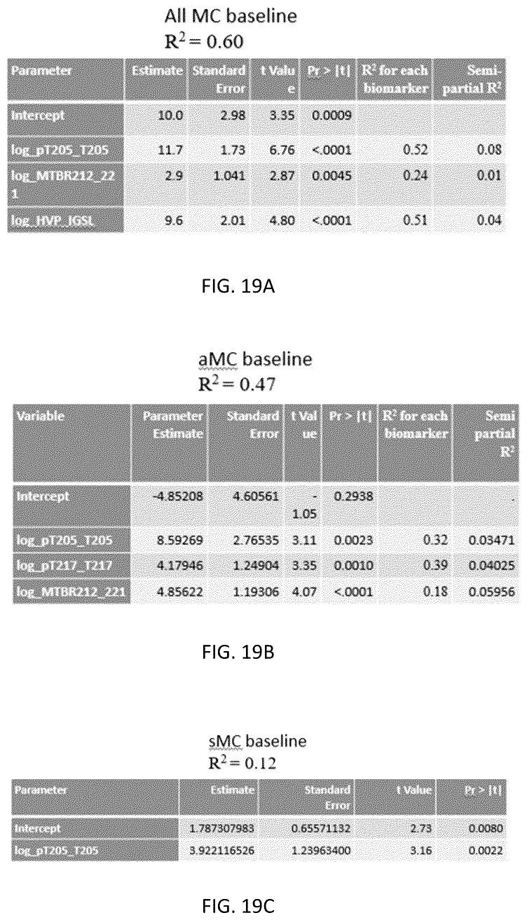

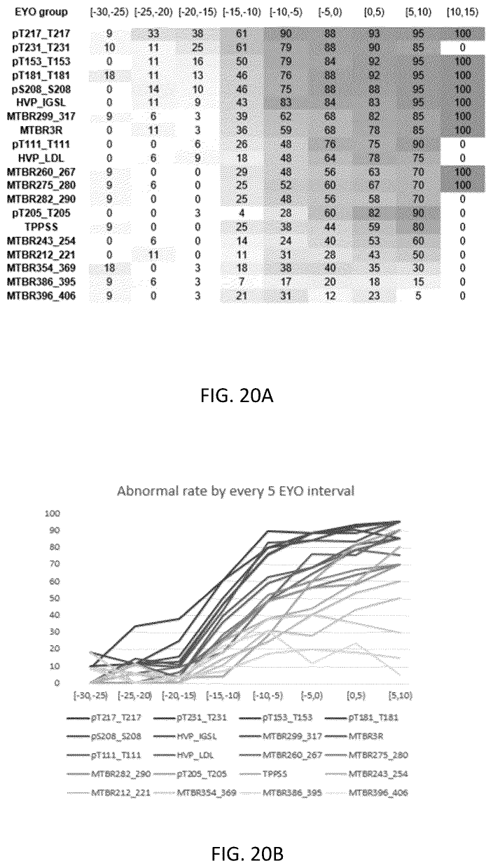

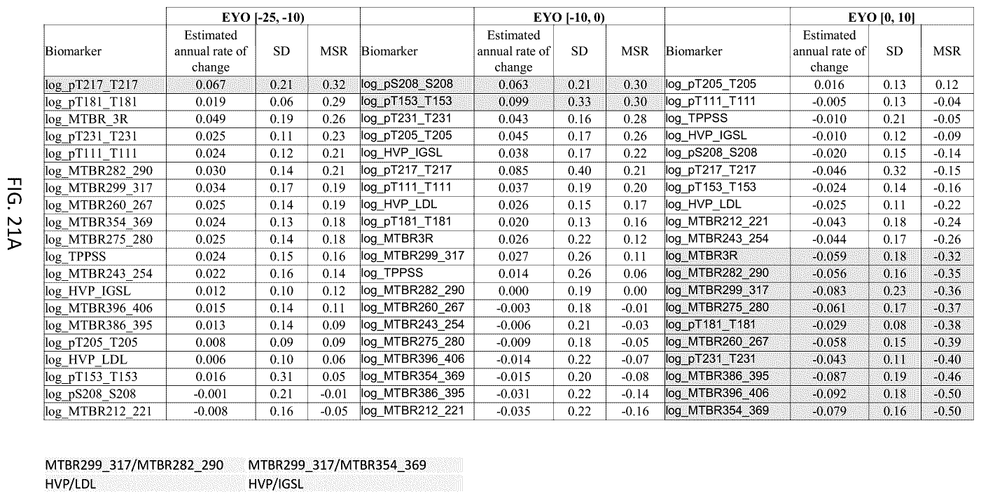

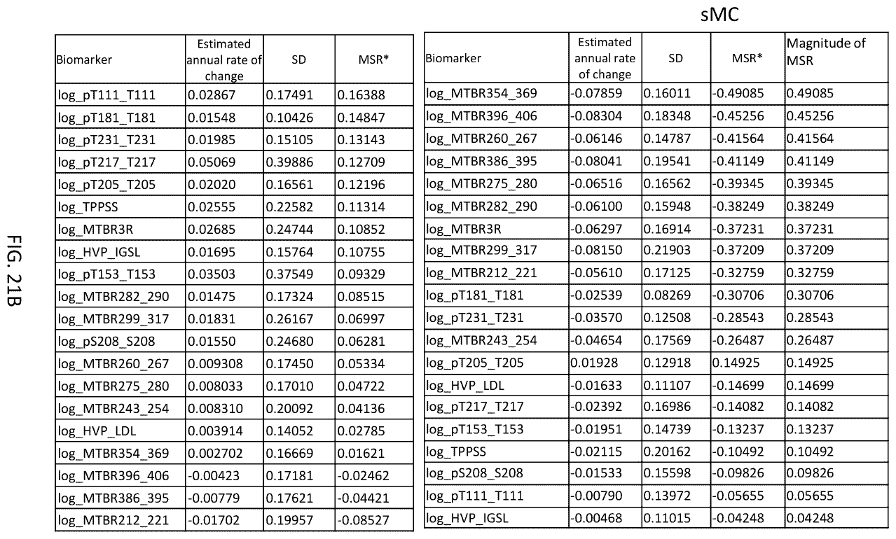

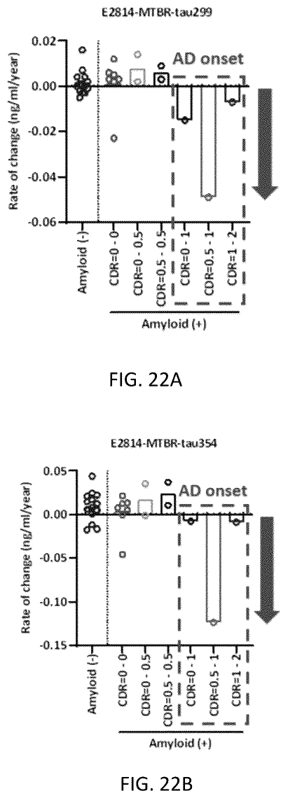

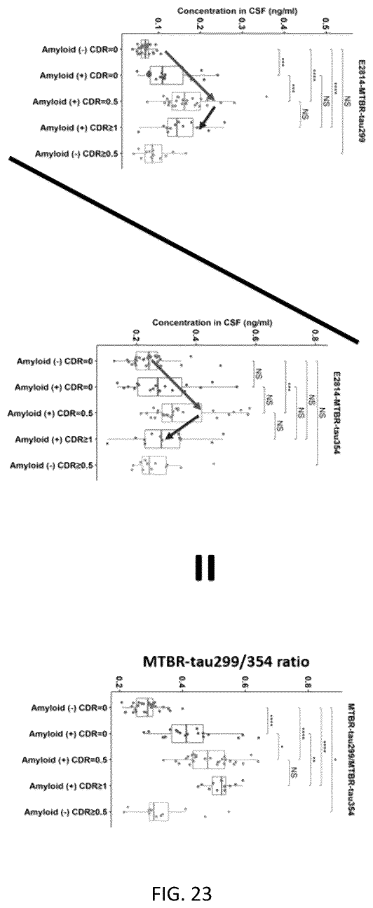

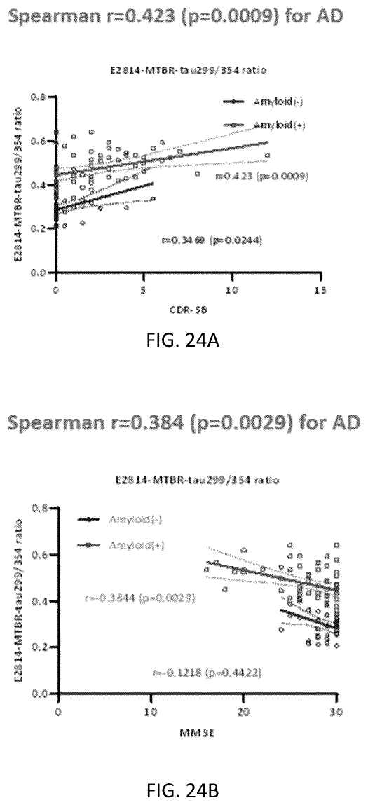

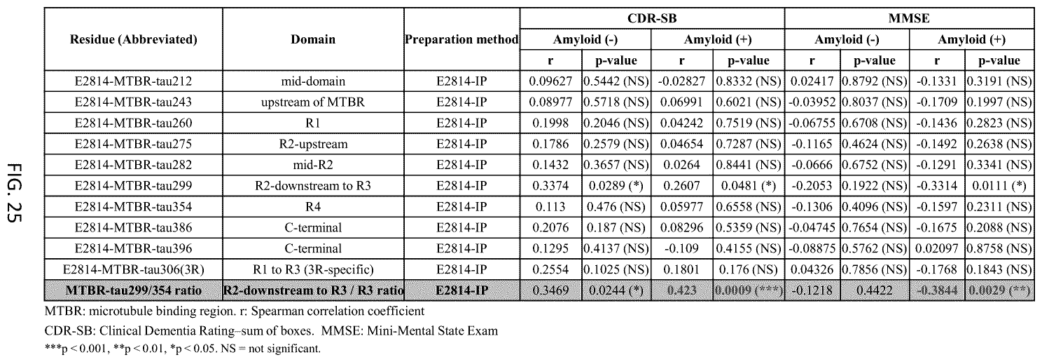

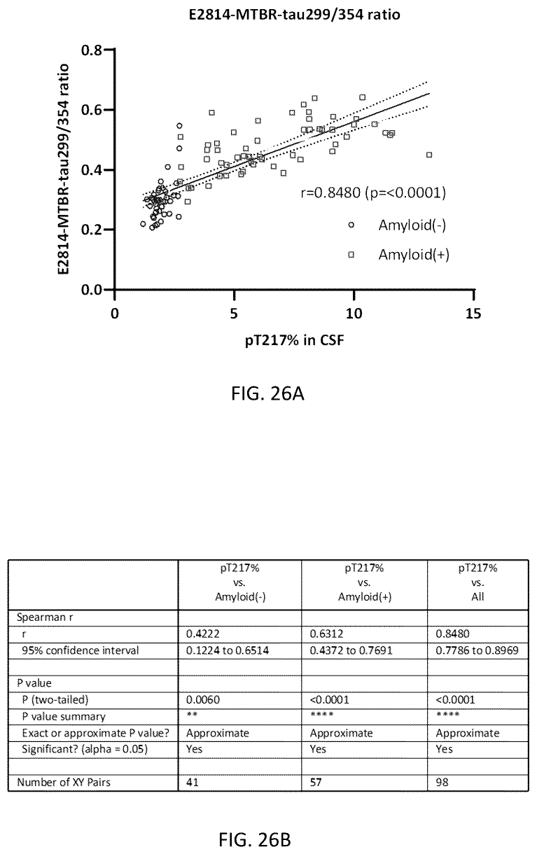

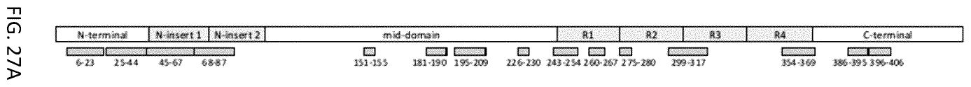

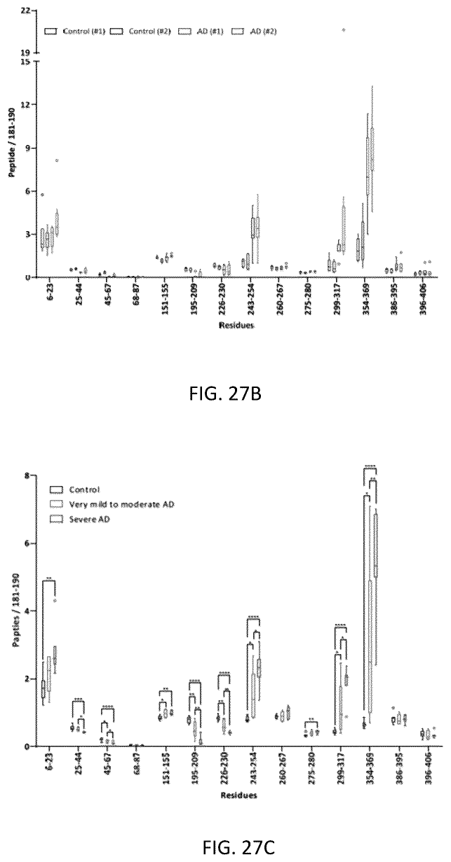

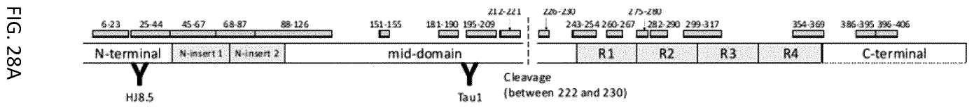

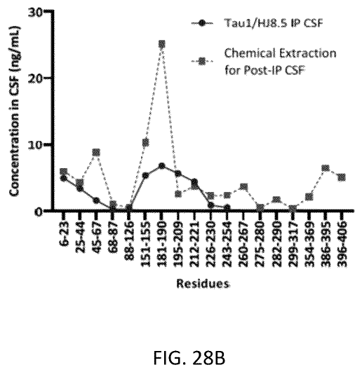

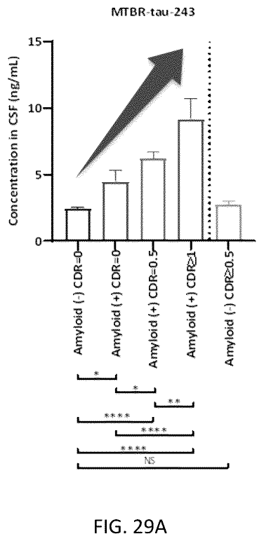

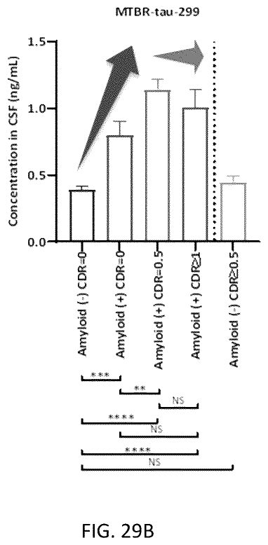

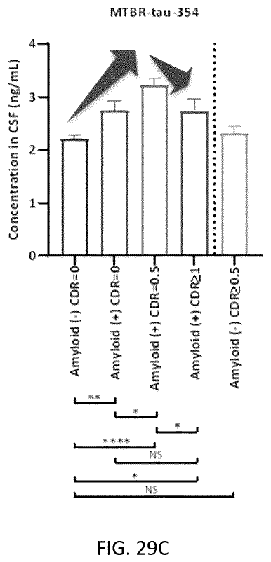

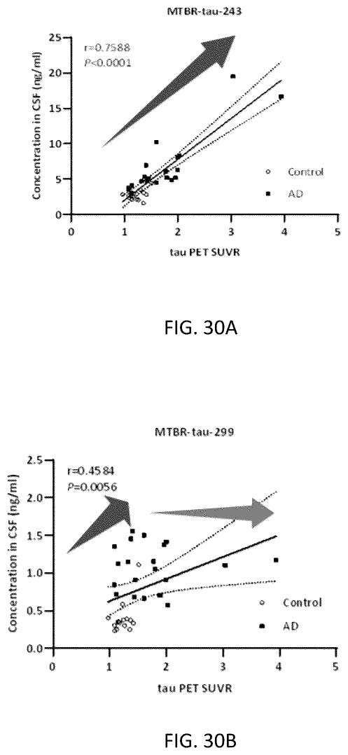

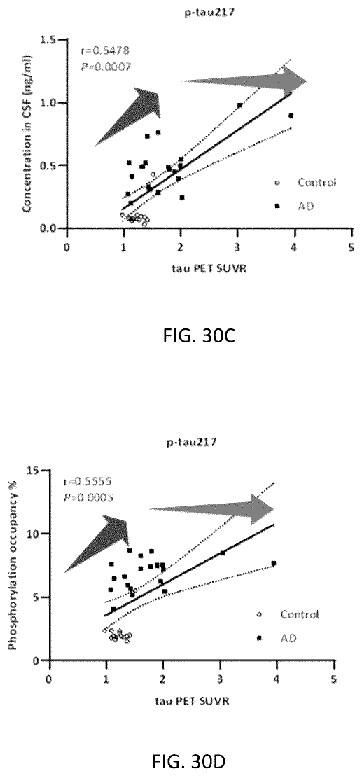

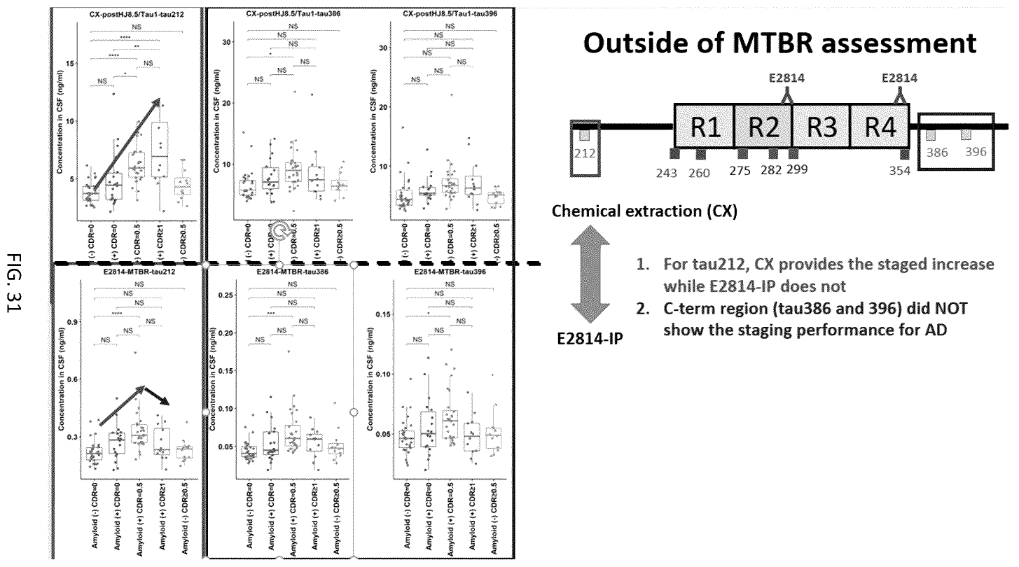

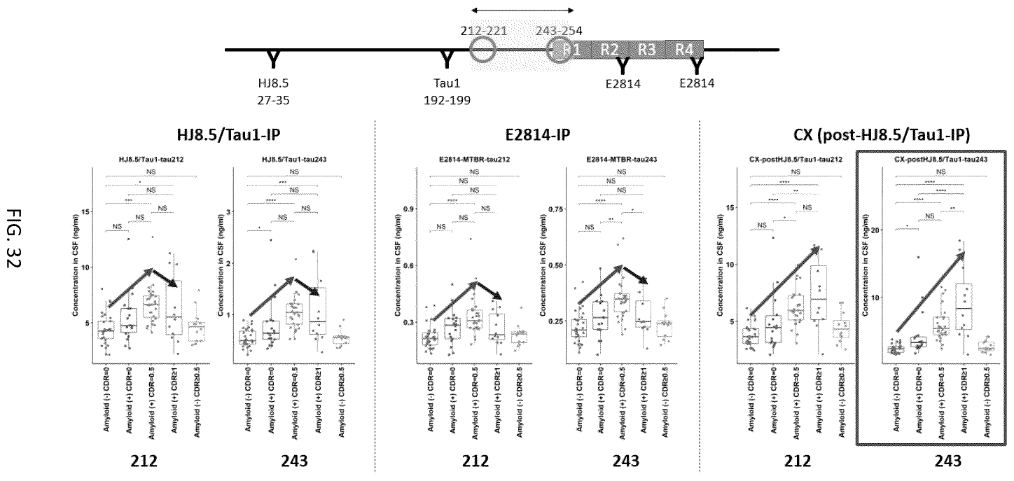

FIGS. 5 and 6 illustrates the dynamic pattern of tau phosphorylation and MTBR-tau levels in an isolated tau sample in relation to years to dementia onset from due to AD. FIG. 20 shows the various tau species abnormal rate by every 5 EYO interval. Phosphorylation levels at T217 that significantly deviate from the mean first occur about 21 years prior to onset of dementia due to AD. The change of MTBR-tau299 in R2 occur about 22 years prior to onset, close to the first detection of change in p-tau217 occupancy. The increase of MTBR-tau354 in R4 saturates in late clinical stages potentially due to the deposition into brain tangles. Notably, the ratio of MTBR-tau299/354 recapitulating tau pathophysiology highly correlates with pT217 occupancy (i.e. about 21 years prior to onset of dementia due to AD). The rate of change for MTBR-tau3R begins to increase about 20 years prior to onset of dementia due to AD and highly correlates with AD progression. The rate of change for MTBR-tau243 begins to increase about 15 years prior to onset of dementia due to AD. Changes in MTBR-tau212 levels are highly correlated with AD progression. Phosphorylation levels at T205 that significantly deviate from the mean first occur about 13 years prior to onset of dementia due to AD.

In one example, a method of the present disclosure comprises providing a biological sample obtained from a subject and (ai) measuring phosphorylation occupancy at residue T205 of tau and measuring MTBR-tau299/MTBR-tau354 ratio, and optionally measuring MTBR-tau212, phosphorylation occupancy at residue T217 MTBR-tau243, and/or MTBR-tau3R, in a blood sample or a CSF sample obtained from the subject, or (aii) measuring phosphorylation occupancy at residue T205 of tau, measuring phosphorylation occupancy at residue T217 of tau, and measuring MTBR-tau212, in a blood sample or a CSF sample obtained from the subject; (b) calculating time to dementia onset using the measurements of (ai) or (aii), wherein time to dementia onset is time in years to a Clinical Dementia Rating greater than zero; and (c) determining the subject as being about 10 to about 25 years, or about 10 to about 20 years from the onset of dementia due to AD when tau phosphorylation at T217 and/or MTBR-tau299/MTBR-tau354 ratio is about 1.5σ or more above the mean and tau phosphorylation at T205 is below about 1.5σ above the mean, where a is the standard deviation defined by the normal distribution of tau phosphorylation at T217 and T205, MTBR-tau299/MTBR-tau354 ratio and MTBR-tau212 measured in a control population without brain amyloid plaques as measured by PET imaging and/or Aβ42/40 measurement in CSF. In various embodiments, tau phosphorylation at T217 and/or MTBR-tau299/MTBR-tau354 ratio may be about 1.3σ, about 1.35σ, about 1.4σ, about 1.45σ, about 1.56, about 1.6σ, about 1.7σ, about 1.8σ, about 1.9σ, about 2σ, or above 2σ above the mean of a control population. In other embodiments, tau phosphorylation at T217 and/or MTBR-tau299/MTBR-tau354 ratio may be about 1.85σ, about 1.9σ, about 1.95σ, about 2σ, about 2.1σ, about 2.2σ, about 2.3σ, about 2.4σ, about 2.5a or above 2.5a above the mean of a control population. In each of the above embodiments, tau phosphorylation at T205 may be at the mean or below about 1.3σ, about 1.35σ, about 1.4σ, about 1.45σ, about 1.56, about 1.51σ, about 1.55σ, about 1.66, about 1.7σ, about 1.8σ, about 1.9σ, about 2.0σ, above the mean of a control population. Alternatively, tau phosphorylation at T205 may be about 2.0σ, about 2.05σ, about 2.1σ, about 2.2σ, about 2.3σ, about 2.4σ, about 2.5σ, or below 2.5a above the mean of a control population. In a further example, tau phosphorylation at T217 and/or MTBR-tau299/MTBR-tau354 ratio about 2a or more above the mean of a control population and tau phosphorylation at T205 may be about below 2a or less above the mean of a control population. In addition to using a threshold (e.g. at least 1 standard deviation above or below the mean), in some embodiment the extent of change of absolute value above or below the mean, or rate of change over time may be used to classify a subject. In still further embodiments, measured levels of tau phosphorylation at T205 and/or T217 and the MTBR-tau299/MTBR-tau354 ratio value and/or MTBR-tau212 value may be used in various mathematical operations to improve the predictive power compared to each by itself. For instance, ratio(s) may be calculated from the measured phosphorylation levels. Mathematical operations other than a ratio may also be used.

In another example, a method of the present disclosure comprises (a) providing a first and a second biological sample obtained from a subject, wherein “first” and “second” refer to the order in which the samples were collected, and measuring tau species as discussed above; (b) calculating the change in the site-specific phosphorylation at each residue measured and the change in MTBR-tau299/MTBR-tau354 ratio value and optionally MTBR-tau212 value; and (c) diagnosing the stage of a subject's AD when the phosphorylation level T217 and/or MTBR-tau299/MTBR-tau354 ratio decreases or stays the same and the phosphorylation level at T205 increases. The first and the second isolated tau samples may be collected days, weeks, or months apart. Typically, tau phosphorylation at the specific sites recited in (a)(i), (a)(ii) or (a)(iii) will also be about 1.5- or above for both samples and, where—is the standard deviation defined by the normal distribution tau phosphorylation at T217 and T205, T181 and T205, or T181, T205 and T217 measured in a control population without brain amyloid plaques as measured by PET imaging and/or Aβ42/40 measurement in CSF. In still further embodiments, measured levels of tau phosphorylation at the specific sites and MTBR-tau species value recited in (a)(i), (a)(ii) may be used in various mathematical operations to improve the predictive power compared to each by itself. For instance, ratio(s) may be calculated from the measured phosphorylation levels. Mathematical operations other than a ratio may also be used.

Methods for measuring tau phosphorylation and MTBR-tau are described in Section II, and incorporated into this section by reference. A skilled artisan will appreciate, however, that the absolute value may vary depending upon the protocol and the source/specifications of internal standards used for absolute quantitation.

In some embodiments of the above, processing a blood sample or a CSF sample from the subject to obtain a first population of enriched tau species and a depleted sample may comprise contacting the blood sample or the CSF sample with an epitope-binding agent the specifically binds to an epitope within the N-terminus of tau, or contacting the blood sample or the CSF sample with an epitope-binding agent the specifically binds to an epitope within the mid-domain of tau, or contacting the blood sample or the CSF sample with a first epitope-binding agent that specifically binds to an epitope within the N-terminus of tau and with a second epitope-binding agent that specifically binds to an epitope within the mid-domain of tau. The first and second epitope-binding agents may be used sequentially or in combination. In some examples, the epitope-binding agent that specifically binds to an epitope within the N-terminus of tau is HJ8.5 or another epitope-binding agent that specifically binds the same epitope as HJ8.5. In some examples, the epitope-binding agent that specifically binds to an epitope within the mid-domain of tau is Tau1 or another epitope-binding agent that specifically binds the same epitope as Tau1.

In other embodiments of the above, processing the depleted sample to obtain a second population of enriched tau species may comprise performing a chemical extraction step to enrich for MTBR-tau species. In certain embodiments, the chemical extract step may comprises admixing an acid to precipitate proteins of the depleted sample, optionally wherein the acid is perchloric acid, and wherein the MTBR-tau species are in the supernatant after removal of the precipitated proteins. Alternatively, processing the depleted sample to obtain a second population of enriched tau species may comprise contacting the depleted sample with an epitope-binding agent that specifically binds to at least one epitope within the MTBR of tau. In certain embodiments, the epitope-binding agent may be 77G7, RD3, RD4, UCB1017, or PT76 described in Vandermeeren et al., J Alzheimers Dis, 2018, 65:265-281, or E2814 or 7G6 described in Roberts et al., Acta Neuropathol Commun, 2020, 8: 13, or antigen-binding fragments of 77G7, RD3, RD4, UCB1017, PT76, E2814 or 7G6, or other epitope-binding agents that specifically bind the same epitopes as 77G7, RD3, RD4, UCB1017, PT76, E2814 or 7G6.

In still other embodiments of the above, processing a blood sample or a CSF sample from the subject to obtain a first population of enriched tau species and a depleted sample may comprise contacting the blood sample or the CSF sample with an epitope-binding agent the specifically binds to an epitope within the N-terminus of tau, or contacting the blood sample or the CSF sample with an epitope-binding agent the specifically binds to an epitope within the mid-domain of tau, or contacting the blood sample or the CSF sample with a first epitope-binding agent that specifically binds to an epitope within the N-terminus of tau and with a second epitope-binding agent that specifically binds to an epitope within the mid-domain of tau; and processing the depleted sample to obtain a second population of enriched tau species comprises performing a chemical extraction step to enrich for MTBR-tau species or contacting the depleted sample with an epitope-binding agent that specifically binds to at least one epitope within the MTBR of tau. The first and second epitope-binding agents may be used sequentially or in combination. In certain examples, the epitope-binding agent that specifically binds to an epitope within the N-terminus of tau is HJ8.5 or another epitope-binding agent that specifically binds the same epitope as HJ8.5; and the epitope-binding agent that specifically binds to an epitope within the mid-domain of tau is Tau1 or another epitope-binding agent that specifically binds the same epitope as Tau1. The chemical extract step may comprise admixing an acid to precipitate proteins of the depleted sample, optionally wherein the acid is perchloric acid, and wherein the MTBR-tau species are in the supernatant after removal of the precipitated proteins. The epitope-binding agent that specifically binds to at least one epitope within the MTBR of tau may be 77G7, RD3, RD4, UCB1017, or PT76 described in Vandermeeren et al., J Alzheimers Dis, 2018, 65:265-281, or E2814 or 7G6 described in Roberts et al., Acta Neuropathol Commun, 2020, 8: 13, or antigen-binding fragments of 77G7, RD3, RD4, UCB1017, PT76, E2814 or 7G6, or other epitope-binding agents that specifically bind the same epitopes as 77G7, RD3, RD4, UCB1017, PT76, E2814 or 7G6.

### IV. Methods of Treatment

The present disclosure also encompasses the use of a time to dementia onset measurement described herein to stage a subject's disease progression; to stage a subject's brain pathology; to select a diagnostic agent for a subject; and to select a therapeutic agent, or a class of therapeutic agents, for a subject that is tailored to the subject's disease stage and underlying disease pathology. Accordingly, another aspect of the present disclosure is a method for treating a subject, the method comprising administering to the subject the therapeutic agent or the diagnostic agent selected for the subject given the subject's time to dementia onset measurement.

The terms “treat,” “treating,” or “treatment” as used herein, refers to the provision of medical care by a trained and licensed professional to a subject in need thereof. The medical care may be a diagnostic test, a therapeutic treatment, and/or a prophylactic or preventative measure. The object of therapeutic and prophylactic treatments is to prevent or slow down (lessen) an undesired physiological change or disease/disorder. Beneficial or desired clinical results of therapeutic or prophylactic treatments include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented. In some embodiments, a subject receiving treatment is asymptomatic. An “asymptomatic subject,” as used herein, refers to a subject that does not show any signs or symptoms of AD. In other embodiments, a subject may exhibit signs or symptoms of AD (e.g., memory loss, misplacing things, changes in mood or behavior, etc.,) but not show sufficient cognitive or functional impairment for a clinical diagnosis of dementia due to Alzheimer's disease. A symptomatic or an asymptomatic subject may have Aβ amyloidosis; however, prior knowledge of Aβ amyloidosis is not a requisite for treatment. In still further embodiments, a subject may be diagnosed as having AD. In any of the aforementioned embodiments, a subject may carry one of the gene mutations known to cause dominantly inherited Alzheimer's disease. In alternative embodiments, a subject may not carry a gene mutation known to cause dominantly inherited Alzheimer's disease.

In one embodiment, a method for treating a subject as described above may comprise providing a biological sample obtained from a subject and ai) measuring phosphorylation occupancy at residue T205 of tau and measuring MTBR-tau299/MTBR-tau354 ratio, and optionally measuring MTBR-tau212, phosphorylation occupancy at residue T217 MTBR-tau243, and/or MTBR-tau3R, in a blood sample or a CSF sample obtained from the subject, or (aii) measuring phosphorylation occupancy at residue T205 of tau, measuring phosphorylation occupancy at residue T217 of tau, and measuring MTBR-tau212; and (b) administering a pharmaceutical composition to the subject when the measured level(s) significantly deviate from the mean in a control population without brain amyloid plaques as measured by PET imaging and/or Aβ42/40 measurement in CSF.

In another embodiment, a method for treating a subject as described above may comprise (a) providing a first and a second biological sample obtained from a subject and measuring in each sample ai) measuring phosphorylation occupancy at residue T205 of tau and measuring MTBR-tau299/MTBR-tau354 ratio, and optionally measuring MTBR-tau212, phosphorylation occupancy at residue T217 MTBR-tau243, and/or MTBR-tau3R, in a blood sample or a CSF sample obtained from the subject, or (aii) measuring phosphorylation occupancy at residue T205 of tau, measuring phosphorylation occupancy at residue T217 of tau, and measuring MTBR-tau212; (b) calculating time to dementia onset using the measurements or change in measurements of (ai) or (aii), wherein time to dementia onset is time in years to a Clinical Dementia Rating greater than zero; and (c) administering a pharmaceutical composition to the subject when the calculated measurements or change(s) significantly deviate from the mean in a control population without brain amyloid plaques as measured by PET imaging and/or Aβ42/40 measurement in CSF. “Significantly deviate from the mean” refers to values that are at least 1 standard deviation, preferably at least 1.3 standard deviations, more preferably at least 1.5 standard deviations or even more preferably at least 2 standard deviations, above or below the mean (i.e., 16, 1.36, 1.56, or 1.56, respectively, where a is the standard deviation defined by the normal distribution measured in a control population without brain amyloid plaques as measured by PET imaging and/or Aβ42/40 measurement in CSF). In addition to using a threshold (e.g. at least 1 standard deviation above or below the mean), in some embodiment the extent of change above or below the mean may be used as criteria for treating a subject.

Alternatively or in addition to using a measurement of site-specific tau phosphorylation, MTBR-tau species, in any of the above embodiments, a ratio calculated from the measured level(s), may be used. Mathematical operations other than a ratio may also be used. For instance, the examples use site-specific tau phosphorylation values in various statistical models (e.g., linear regressions, LME curves, LOESS curves, etc.) in conjunction with other known biomarkers (e.g. APOE ε4 status, age, sex, cognitive test scores, functional test scores, etc.).

Many imaging agents and therapeutic agents contemplated for, or used with, subjects at risk of developing Aβ amyloidosis or AD, subjects diagnosed as having Aβ amyloidosis, subjects diagnosed as having a tauopathy, or subjects diagnosed as having AD, target a specific pathophysiological change. For instance, Aβ targeting therapies are generally designed to decrease Aβ production, antagonize Aβ aggregation or increase brain Aβ clearance; tau targeting therapies are generally designed to alter tau phosphorylation patterns, antagonize tau aggregation, or increase NFT clearance; a variety of therapies are designed to reduce CNS inflammation or brain insulin resistance; etc. The efficacy of these various agents can be improved by administering the agents to subjects that staged by methods disclosed herein.

In an exemplary embodiment, the efficacy of imaging agents and therapeutic agents contemplated for, or used with, subjects at risk of developing Aβ amyloidosis or AD, subjects diagnosed as having Aβ amyloidosis, subjects diagnosed as having a tauopathy, or subjects diagnosed as having AD (collectively referred to herein as “Aβ and tau therapies”) can be improved by administering the Aβ or tau therapy to subjects that have certain tau phosphorylation levels at T205 and/or T217, and/or MTBR-tau299/MTBR-tau354 ratio, and optionally measuring MTBR-tau212 and/or MTBR-tau3R and/or MTBR-243 as measured by methods disclosed herein and illustrated in the figures and examples.

For instance, in embodiments where the time to dementia onset measurement is about 20 years, about 25 years, or about 30 years or more, suitable therapeutics may be a primary preventative therapy that prevents pathological amyloid deposition (i.e., amyloid deposition greater than would be expected for a subject's age). Non-limiting examples include therapeutic agents that decreases Aβ production, prevents or antagonizes Aβ aggregation, or increases brain Aβ clearance including but not limited to gamma-secretase inhibitors, beta-secretase inhibitors, passive immunotherapies (including but not limited to an anti-Aβ antibody, an anti-tau antibody, or an anti-ApoE antibody), active immunotherapies.

In embodiments where the time to dementia onset measurement is about 25 years to about 15 years, or about 20 years to about 15 years, suitable therapeutics may be a secondary preventative therapy that prevents further pathological amyloid-beta deposition or decreases a subject's existing amyloid-beta plaque load. Non-limiting examples include therapeutic agents that decreases Aβ production, prevents or antagonizes Aβ aggregation, or increases brain Aβ clearance including but not limited to gamma-secretase inhibitors, beta-secretase inhibitors, and passive immunotherapies (including but not limited to an anti-Aβ antibody, an anti-tau antibody, or an anti-ApoE antibody).

In embodiments where the time to dementia onset measurement is about 15 years or less, suitable therapeutics may be a secondary preventative therapy that prevent or antagonize tau aggregation or that target neurofibrillary tangles, in addition to those that prevent further pathological amyloid-beta deposition or decrease a subject's existing amyloid-beta plaque load. Non-limiting examples include therapeutic agents that prevent or antagonize tau aggregation or that target neurofibrillary tangles include tau protein aggregation inhibitors, kinase inhibitors, phosphatase activators, passive immunotherapies (including but not limited to anti-tau antibodies).

Non-limiting examples of anti-Aβ antibodies include solanezumab (LY2062430; Eli Lilly), aducanumab (BI-1B037; Biogen), crenezumab (MABT102A, RG7412, Genentech and Roche), gantenerumab (R04909832, RG14502; Roche), bapinezumab (Janssen and Pfizer), BAN2401 (Eisai), LY3002813 (Lilly), R07126209 (Roche), AAB-003, and GK933776.

Non-limiting example of anti-tau antibodies include semorinemab (AC Immune and Genentech), ABBV-8E12 (Abbvie), B11B092 (Biogen), B11B076 (Biogen), LY3303560 (Lilly), R07105705 (Roche/Genentech), JNJ-63733657 (Janssen), Lu AF87908 (Lundbeck).

Non-limiting example of anti-ApoE antibodies include HJ6.3, HAE-4 (WUSTL, Denali Therapeutics).

Non-limiting examples of therapeutic agents also include a cholinesterase inhibitor, an N-methyl D-aspartate (NMDA) antagonist, an antidepressant (e.g., a selective serotonin reuptake inhibitor, an atypical antidepressant, an aminoketone, a selective serotonin and norepinephrine reuptake inhibitor, a tricyclic antidepressant, etc.), a gamma-secretase inhibitor, a beta-secretase inhibitor, an anti-Aβ antibody (including antigen-binding fragments, variants, or derivatives thereof), an anti-tau antibody (including antigen-binding fragments, variants, or derivatives thereof), an anti-TREM2 antibody (including antigen-binding fragments, variants or derivatives thereof, a TREM2 agonist, stem cells, dietary supplements (e.g. lithium water, omega-3 fatty acids with lipoic acid, long chain triglycerides, genistein, resveratrol, curcumin, and grape seed extract, etc.), an antagonist of the serotonin receptor 6, a p38alpha MAPK inhibitor, a recombinant granulocyte macrophage colony-stimulating factor, a passive immunotherapy, an active vaccine (e.g. CAD106, AF20513, etc.), a tau protein aggregation inhibitor (e.g. TRx0237, methylthionimium chloride, etc.), a therapy to improve blood sugar control (e.g., insulin, exenatide, liraglutide pioglitazone, etc.), an anti-inflammatory agent, a phosphodiesterase 9A inhibitor, a sigma-1 receptor agonist, a kinase inhibitor, a phosphatase activator, a phosphatase inhibitor, an angiotensin receptor blocker, a CB1 and/or CB2 endocannabinoid receptor partial agonist, a p-2 adrenergic receptor agonist, a nicotinic acetylcholine receptor agonist, a 5-HT2A inverse agonist, an alpha-2c adrenergic receptor antagonist, a 5-HT 1A and 1D receptor agonist, a Glutaminyl-peptide cyclotransferase inhibitor, a selective inhibitor of APP production, a monoamine oxidase B inhibitor, a glutamate receptor antagonist, a AMPA receptor agonist, a nerve growth factor stimulant, a HMG-CoA reductase inhibitor, a neurotrophic agent, a muscarinic M1 receptor agonist, a GABA receptor modulator, a PPAR-gamma agonist, a microtubule protein modulator, a calcium channel blocker, an antihypertensive agent, a statin, and any combination thereof. In an exemplary embodiment, a pharmaceutical composition may comprise a kinase inhibitor. Suitable kinase inhibitors may inhibit a thousand-and-one amino acid kinase (TAOK), CDK, GSK-3β, MARK, CDK5, or Fyn. In another exemplary embodiment, a pharmaceutical composition may comprise a phosphatase activator. As a non-limiting example, a phosphatase activator may increase the activity of protein phosphatase 2A. In some embodiments the treatment is a pharmaceutical composition comprising a tau targeting therapy, including but not limited to active pharmaceutical ingredients that alter tau phosphorylation patterns, antagonize tau aggregation, or increase clearance of pathological tau isoforms and/or aggregates. In some embodiments, the treatment is an anti-Aβ antibody, an anti-tau antibody, an anti-TREM2 antibody, a TREM2 agonist, a gamma-secretase inhibitor, a beta-secretase inhibitor, a kinase inhibitor, a phosphatase activator, a vaccine, or a tau protein aggregation inhibitor.

Non-limiting examples of classes of therapeutic agents and specific examples are also presented in the table below.

| | |
|---|---|
| Aβ targeting therapies are generally | agents that prevent tau |
| designed to | aggregation or that target |
| decrease Aβ production, | NFTs are generally |
| antagonize Aβ aggregation or | designed to |
| increase brain Aβ clearance | alter tau phosphorylation |
| specific types of agents that are | patterns, |
| known to decrease Aβ production, | antagonize tau |
| antagonize Aβ aggregation or | aggregation, or |
| increase brain Aβ | increase neurofibrillary |
| clearance: | tangle (NFT) clearance. |
| gamma-secretase inhibitors | specific types of agents |
| beta-secretase inhibitors | that are known to decrease |
| anti-Aβ antibodies | alter tau phosphorylation |
| passive immunotherapies | patterns, antagonize tau |
| active immunotherapies | aggregation, or increase |
| | nuerofibrillary tangle |
| | clearance: |
| | include tau protein |
| | aggregation inhibitors, |
| | kinase inhibitors, |
| | phosphatase activators, |
| | anti-tau antibodies |
| | passive immunotherapies, |
| | and |
| | active immunotherapies. |
| Modification of Amyloid-beta | Inhibit tau phosphorylation: |
| generation: | lithium |
| AZD3293 (β-secretase inhibitor) | valproic acid |
| MK-8931(β-secretase inhibitor) | tau aggregation inhibitor: |
| Tarenflurbil (gamma-secretase | methylthioninium |
| inhibitors and modulators) | |
| Semagacestat (gamma-secretase | |
| inhibitors and modulators) | |
| Modification of Amyloid-beta | |
| aggregation: | |
| solanezumab (anti-Aβ antibody) | |
| bapineuzumab (anti-Aβ antibody) | |
| aducanumab (anti-Aβ antibody) | |
| Inhibition of Aβ production by | Enhancement of microtubule |
| β-secretase (BACE) inhibitors: | stabilization by tau stabilizers |
| BI 1181181 | Epothilone D |
| RG7129 | TPI 287 |
| LY2811376 | Prevention of tau aggregation |
| LY2886721 | by tau aggregation inhibitors |
| E2609 | Rember |
| AZD3293 | TRxo237 |
| CNP520 | Enhancement of phosphorylated- |
| JNJ-54861911 | tau clearance by active |
| verubecestat (MK-8931) | immunotherapy |
| Inhibition of Aβ production by | AADvac-1 |
| y-secretase inhibitors: | ACI-35 |
| semagacestat | |
| Avagacestat | |
| EVP-0962 | |
| NIC5-15, pinitol | |
| Enhancement of Aβ clearance by active | |
| immunotherapy | |
| AN-1792 | |
| CAD106 | |
| ACC-001 (vanutide cridificar) | |
| Affitope AD02 | |
| Enhancement of Aβ clearance by | |
| passive immunotherapy | |
| Bapineuzumab (anti-Aβ antibody) | |
| AAB-003 (anti-Aβ antibody) | |
| GSK933776 (anti-Aβ antibody) | |
| Solanezumab (anti-Aβ antibody) | |
| Crenezumab (anti-Aβ antibody) | |
| Gantenerumab (anti-Aβ antibody) | |
| BAN2401 (anti-Aβ antibody) | |
| Aducanumab (anti-Aβ antibody) | |
| | PP2A activators |
| | Memantine |
| | Sodium selenite |
| | GSK3β Inhibitors |
| | Tideglusib |
| | Lithium chloride |
| | Acetylation inhibitors |
| | Salsalate |
| | OGA inhibitors |
| | MK-8719 |
| | Aggregation Inhibitors |
| | LMTX |
| | Curcumin |
| | Microtubule stabilizers |
| | Epithilone D |
| | NAP |
| | TPI 287 |
| | PDE4 inhibitors |
| | BPN14770 |
| Inhibition of Aβ deposition/ | |
| enhancement of Aβ clearance | |
| by passive immunization | |
| Anti-ApoE antibodies | |
| Agent that disaggregates Aβ | Reduce tau production |
| NPT088 | Ionis MAPT Rx |
| | Nilotinib (kinase inhibitor) |
| | Reduce tau phosphorylation |
| | Nicotinamide (HDAC |
| | Inhibitor) |
| | AZD0530 (kinase inhibitor) |
| | Passive Immunotherapy |
| | LY3303560 (anti-tau Ab) |
| | ABBV-8E12 (anti-tau Ab) |
| | BIIB092 (anti-tau Ab) |
| | 63733657 (anti-tau Ab) |
| | RO7105705 (anti-tau Ab) |
| | NPT088 |
| | Active immunotherapy |
| | AADvac1 |
| | ACI-35 |
| | Tau protein aggregation inhibitor |
| | Methylene Blue |
| | morphomers |

For instance, when tau phosphorylation at T217 and MTBR-tau299/MTBR-tau354 ratio value is about 1.5σ or more above the mean of a control population and tau phosphorylation at T205 is near the mean or below about 1.5σ or more above the mean of a control population, preferred therapeutic agents may include those designed to prevent a subject from becoming amyloid positive (e.g., amyloid targeting therapies designed to decrease Aβ production, antagonize Aβ aggregation, etc.). As another example, when tau phosphorylation at T217 and MTBR-tau299/MTBR-tau354 ratio value is about 1.5σ or more above the mean of a control population and tau phosphorylation at T205 is about 1.5σ or more above the mean of a control population, preferred therapeutic agents may include those designed to prevent amyloid deposition from increasing or reduce a subject's existing plaque load. As another example, when tau phosphorylation at T217, and MTBR-tau299/MTBR-tau354 ratio value and T205 are about 1.5σ or more above the mean of a control population, preferred therapeutic agents may include those designed to prevent amyloid deposition from increasing, reduce a subject's existing plaque load, prevent tau aggregation, or target NFTs. As another example, when tau phosphorylation at T217, and MTBR-tau299/MTBR-tau354 ratio value and T205 is about 1.5σ or more above the mean of a control population, and tau phosphorylation at T217 and MTBR-tau299/MTBR-tau354 ratio value is plateauing or decreasing, and total tau and/or tau phosphorylation at T205 is increasing, preferred therapeutic agents may include those designed to prevent amyloid deposition from increasing, reduce a subject's existing plaque load, prevent tau aggregation, or target NFTs, as well as those specific for subjects with AD. The details disclosed herein can similarly be used to administer therapeutic agents designed for other targets (e.g., CNS inflammation, ApoE, etc.), including but not limited to those identified in the preceding paragraphs.

### EXAMPLES

The following examples illustrate various iterations of the invention. It should be appreciated by those of skill in the art that the techniques disclosed in the examples that follow represent techniques discovered by the inventors to function well in the practice of the invention. Those of skill in the art should, however, in light of the present disclosure, appreciate that changes may be made in the specific embodiments that are disclosed and still obtain a like or similar result without departing from the spirit and scope of the invention. Therefore, all matter set forth or shown in the accompanying drawings is to be interpreted as illustrative and not in a limiting sense.

### Example 1

Several sample processing methods were developed—an immunoprecipitation method for N-terminal tau and mid-domain tau (IP), described in Sato et al., 2018; a chemical extraction method (CX); and a process combining the IP and CX methods to enrich for MTBR tau (PostIP-CX). The CX and PostIP-CX methods were specifically developed to detect and quantify MTBR tau. An overview of these methods is provided in FIG. 2.

Briefly, CSF (about 475 μL) was mixed with a solution containing ¹⁵N Tau-441(2N4R) Uniform Labeled (approximately 10 μL of 100 pg/μL solution, or approximately 5 μL of a 200 pg/μL solution) as an internal standard. N-terminal tau and mid-domain tau species were immunoprecipitated with Tau1 and HJ8.5 antibodies, and then processed and trypsin digested as described previously (Sato et al., 2018).

For the CX method, CSF (about 475 μL) was mixed with a solution containing ¹⁵N Tau-441(2N4R) Uniform Labeled (approximately 10 μL of 100 pg/μL solution, or approximately 5 μL of a 200 pg/μL solution) as an internal standard. Then, tau was chemically extracted. Highly abundant CSF proteins were precipitated using 25 μL of perchloric acid. After mixing and incubation on ice for 15 minutes, the mixture was centrifuged at 20,000 g for 15 minutes at 4° C., and the supernatant was further purified using the Oasis HLB 96-well μElution Plate (Waters) according to the following steps. The plate was washed once with 300 μL of methanol and equilibrated once with 500 μL of 0.1% FA in water. The supernatant was added to the Oasis HLB 96-well μElution Plate and adsorbed to the solid phase. Then, the solid phase was washed once with 500 μL of 0.1% FA in water. Elution buffer (100 μL; 35% acetonitrile and 0.1% FA in water) was added, and the eluent was dried by Speed-vac. Dried sample was dissolved by 50 μL of trypsin solution (10 ng/μL) in 50 mM TEABC and incubated at 37° C. for 20 hours.

For the PostIP-CX method, the post-immunoprecipitated CSF (i.e., the supernatant remaining after the IP method described above) was processed as described in the CX method.

Following tryptic digestion, all samples were purified by solid phase extraction on C18 TopTip. In this purification process, 5 fmol each of AQUA internal-standard peptide for residues 354-369 (MTBR tau-354) and 354-368 (tau368) was spiked for the differential quantification. Before eluting samples, 3% hydrogen peroxide and 3% FA in water were added to the beads, followed by overnight incubation at 4° C. to oxidize the peptides containing methionine. The eluent was lyophilized and resuspended in 27.5 μL of 2% acetonitrile and 0.1% FA in water prior to MS analysis on nanoAcquity UPLC system coupled to Orbitrap Fusion Lumos Tribrid or Orbitrap Tribrid Eclipse mass spectrometer (Thermo Scientific) operating in PRM mode.

The CX and PostIP-CX methods produced samples comprising MTBR tau detectable and quantifiable by mass spectrometry. Quantifiable signals of MTBR tau were not obtained by the IP method. Although not demonstrated, it is believed alternative methods for detecting and quantifying MTBR tau that have similar sensitivity may also be used.

### Example 2

CSF tau analysis by MS: CSF (455 μL) was mixed with 10 μL of solution containing ¹⁵N Tau-441(2N4R) Uniform Labeled (100 pg/μL) as an internal standard. The tau species consisting primarily of N-terminal to mid-domain regions were immunoprecipitated with Tau1 and HJ8.5 antibodies. Immunoprecipitated tau species were processed and digested as described previously (Sato et al., 2018). Subsequently, 20 μL of ¹⁵N-tau internal standard (100 pg/μL) was spiked into the post-immunoprecipitated CSF. Then, tau was chemically extracted as previously reported (Barthélemy et al., 2016b) with some modifications. Highly abundant CSF proteins were precipitated using 25 μL of perchloric acid. After mixing and incubation on ice for 15 minutes, the mixture was centrifuged at 20,000 g for 15 minutes at 4° C., and the supernatant was further purified using the Oasis HLB 96-well μElution Plate (Waters) according to the following steps. The plate was washed once with 300 μL of methanol and equilibrated once with 500 μL of 0.1% FA in water. The supernatant was added to the Oasis HLB 96-well μElution Plate and adsorbed to the solid phase. Then, the solid phase was washed once with 500 μL of 0.1% FA in water. Elution buffer (100 μL; 35% acetonitrile and 0.1% FA in water) was added, and the eluent was dried by Speed-vac. Dried sample was dissolved by 50 μL of trypsin solution (10 ng/μL) in 50 mM TEABC and incubated at 37° C. for 20 hours.

After incubation for both immunoprecipitated and chemically extracted samples, each tryptic digest was purified by solid phase extraction on C18 TopTip. In this purification process, 5 fmol each of AQUA internal-standard peptide for residues 354-369 (MTBR tau-354) and 354-368 (tau368) was spiked for the differential quantification. Before eluting samples, 3% hydrogen peroxide and 3% FA in water were added to the beads, followed by overnight incubation at 4° C. to oxidize the peptides containing methionine. The eluent was lyophilized and resuspended in 27.5 μL of 2% acetonitrile and 0.1% FA in water prior to MS analysis on nanoAcquity UPLC system coupled to Orbitrap Fusion Lumos Tribrid or Orbitrap Tribrid Eclipse mass spectrometer (Thermo Scientific) operating in PRM mode. Nineteen CSF tau peptides were quantified (Table 1). The schematic procedure of CSF tau analysis is described in FIG. 2A.

Statistical analysis: Differences in biomarker values were assessed with one-way ANOVAs, unless otherwise specified. A two-sided p<0.05 was considered statistically significant and corrected for multiple comparisons using Benjamini-Hochberg false discovery rate (FDR) method with FDR set at 5% (Benjamini and Hochberg, 1995). Spearman correlations were used to assess associations between tau biomarkers and cognitive testing measures and tau PET SUVR.

TABLE 1

| | Tau-441 residues |
|---|---|
| Peptide | (Abbreviated) |
| QEFEVMEDHAGTYGLGDR (SEQ ID NO: 11) | 6-23 |
| DQGGYTMHQDQEGDTDAGLK (SEQ ID NO: 12) | 25-44 |
| ESPLQTPTEDGSEEPGSETSDAK (SEQ ID NO: 13) | 45-67 |
| STPTAEDVTAPLVDEGAPGK (SEQ ID NO: 14) | 68-87 |
| QAAAQPHTEIPEGTTAEEAGIGDTPSLEDEAAGHVTQAR | 88-126 |
| (SEQ ID NO: 15) | |
| IATPR (SEQ ID NO: 16) | 151-155 |
| TPPSSGEPPK (SEQ ID NO: 10) | 181-190 |
| SGYSSPGSPGTPGSR (SEQ ID NO: 17) | 195-209 |
| TPSLPTPPTR (SEQ ID NO: 18) | 212-221 |
| VAVVR (SEQ ID NO: 19) | 226-230 |
| LQTAPVPMPDLK (SEQ ID NO: 3) | 243-254 |
| | (MTBR tau-243) |
| IGSTENLK (SEQ ID NO: 2) | 260-267 |
| VQIINK (SEQ ID NO: 4) | 275-280 |
| LDLSNVQSK (SEQ ID NO: 5) | 282-290 |
| HVPGGGSVQIVYKPVDLSK (SEQ ID NO: 6) | 299-317 |
| | (MTBR tau-299) |
| IGSLDNITHVPGGGNK (SEQ ID NO: 7) | 354-369 |
| | (MTBR tau-354) |
| IGSLDNITHVPGGGN (SEQ ID NO: 8) | 354-368 (tau368) |
| TDHGAEIVYK (SEQ ID NO: 20) | 386-395 |
| SPVVSGDTSPR (SEQ ID NO: 21) | 396-406 |

### Example 3

An additional sample processing method, referred to as “PostIP-IP”, was developed and compared to the PostIP-CX method described in Examples 1 and 2. Exemplary workflows of the PostIP-IP method are provided in FIG. 3 and FIG. 4.

CSF samples obtained from the LOAD100 cohort described in Example 2 were processed by the PostIP-CX method (Example 1) or the PostIP-IP method (this example) and then analyzed by LC-MS as generally described in Example 2.

### Example 4

Advances in methods for identifying and quantitating an increasing number of soluble tau species from plasma and cerebral spinal fluid (CSF) offers the opportunity to better understand the role of tau related pathology in Alzheimer disease (AD). Recent work in dominantly inherited and sporadic AD has demonstrated hyperphosphorylation at specific positions of the tau protein, N-terminal fragments and microtubule binding regions (MTBR) have been associated with different aspects of the AD cascade. However, there is a need to evaluate the potential unique information provided by different soluble tau species. The Dominantly Inherited AD Network (DIAN) study offers the opportunity to explore the association of multiple soluble tau species with disease progression and multiple biomarkers of amyloid and neurodegeneration.

In this example, over 12 species of CSF phosphorylated tau (p-tau) and truncated MTBR species were quantified from 227 mutation carriers (MCs) (152 asymptomatic MCs, 77 symptomatic MCs) and 141 non-carriers enrolled in DIAN. The best combinations of p-tau and MTBR species for predicting estimated years to symptom onset (EYO) were evaluated using linear regression with stepwise selection. The pattern of associations in baseline level and in longitudinal rate of change among tau/p-tau species and other biomarkers were examined using correlation heatmaps and hierarchical clustering.

DIAN is an international, multicenter registry of individuals (mutation carriers and noncarriers; asymptomatic and symptomatic) who are biological adult children of a parent with a known causative mutation for AD in the APP, PSEN1, or PSEN2 genes, in which the individuals are evaluated in a uniform manner at entry and longitudinally thereafter with standard instruments. The standard instruments include: (1) the clinical and cognitive batteries of the Uniform Data Set (UDS) and additional neuropsychological and personality measures; (2) the Alzheimer's Disease Neuroimaging Initiative (ADNI) structural (magnetic resonance imaging, or MRI, functional (18Fluorodeoxyglucose positron emission tomography, or FDG PET), and amyloid imaging (Pittsburgh Compound-B, or PIB) PET protocols; (3) in accordance with the ADNI protocols, collection of biological fluids (blood; CSF) for DNA analysis and assays of putative biomarkers of AD, and (4) uniform histopathological examination of cerebral tissue in individuals who come to autopsy. In DIAN, symptomatic individuals are individuals with a Clinical Dementia Rating greater than zero (CDR>0). The Clinical Dementia Rating is well-known scale used to quantify the severity of symptoms of dementia. Symptom risk in DIAN is defined by EYO. EYO here is defined a parental age of dementia diagnosis minus the current age of the participant. For participants who were symptomatic at baseline, as assessed by a CDR>0, the reported age at actual symptom onset was subtracted from age at each clinical assessment to define EYO.

### Phospho-Tau Detection and Quantification: Sample Processing

Human CSF was pooled from a cohort of 80 participants, including amyloid negative and cognitively normal (CDR=0) controls (n=47, age 60+) and amyloid positive and CDR>0 AD patients (n=33, age 60+). Five and seven pools of 500 μL CSF aliquots were generated from the control and AD groups, respectively. At the time of initial collection, CSF was spun down at 1,000× g for 10 min to remove cell debris and immediately frozen at −80° C. Protease inhibitor cocktail was added during experiments. Tau was immunoprecipitated and desalted as previously described with some modifications (Sato et al., 2018). Briefly, CNBr-activated Sepharose beads (GE Healthcare 17-0430-01) were crosslinked to antibodies Tau1 and HJ8.5, separately at a concentration of 3 mg antibody per gram of beads. Samples were spiked with AQUA peptides (ThermoFisher Scientific) to an amount of 10 fmol phosphorylated and 100 fmol unphosphorylated tau for each sequence of interest per microliter of sample. Tau and p-tau concentration is calculated using these internal standards. Soluble tau was immunoprecipitated in detergent (1% NP-40), chaotropic reagent (5 mM guanidine), and protease inhibitors (Roche Complete Protease Inhibitor Cocktail). Anti-Tau1 and HJ8.5 antibodies conjugated to sepharose beads were diluted 10 and 5-fold, respectively, in inactivated sepharose beads, and 30 μL of 50% slurry of the antibody beads were rotated with the solution for 90 min at room temperature. The beads were washed three times in 25 mM triethyl ammonium bicarbonate buffer (TEABC, Fluka 17902). The bound tau was digested on-beads with 400 ng MS grade trypsin (Promega, V5111) for 16 h at 37° C. Digests were loaded onto TopTip C18 (Glygen, TT2C18.96), desalted, and eluted per manufacturer's instructions. The eluted peptides were dried by vacuum centrifugation (CentriVap Concentrator Labconco) and were resuspended in 25 μL of a solution of 2% acetonitrile and 0.1% formic acid in MS grade water.

Human blood is processed in substantially the same manner as above, though larger amounts of blood may be used (e.g., about 500 μL to about 10 mL).

Additional details may be found in PCT/US2019/030725, the disclosures of which are incorporated herein by reference.

### MTBR-Tau Detection and Quantification: Sample Processing

For detection and quantification of MTBR tau species, CSF was processed as depicted in FIG. 4. Immunoprecipitation with Tau1 and HJ8.5 were as generally described above. E2814 is an anti-tau antibody (Eisai). See, for instance, Roberts et al., Acta Neuropathol Commun, 2020, 8(1): 13. Tryptic digestion and desalting using TopTip C18 was as performed as generally described above. Human blood is processed in substantially the same manner, though larger amounts of blood may be used (e.g., about 0.5 mL to about 10 mL).

### Phospho-Tau and MTBR Tau Detection and Quantification: Mass Spectrometry

A 5 μL aliquot of the peptide resuspension was injected into nano-Acquity LC for MS analysis. The nano-Acquity LC (Waters Corporation, Milford, MA, USA) was fitted with HSS T3 75 μm×100 μm, 1.8 μm column and a flow rate of 0.5 μL/min of a gradient of solution A and B was used to separate the peptides. Solution A was composed of 0.1% formic acid in MS grade water and solution B was composed of 0.1% formic acid in acetonitrile. Peptides were eluted from the column with a gradient of 2%-20% of solution B in 28 min, then 20%-40% solution B for another 13 min before ramping up to 85% solution B in another 3 min to clean the column. The Orbitrap Fusion Lumos was equipped with a Nanospray Flex electrospray ion source (Thermo Fisher Scientific, San Jose, CA, USA). Peptide ions sprayed from a 10 μm SilicaTip emitter (New Objective, Woburn, MA, USA) into the ion source were targeted and isolated in the quadrupole. These were fragmented by HCD and ion fragments were detected in the Orbitrap (resolution of 30,000 or 60,000, mass range 150-1,200 m/z). Monitoring of hydrophilic peptides (SSRcalc<9, all without leucine) for peptide profiling was performed on a HSS T3 300 μm×100 μm, 1.8 mm column at a flow rate of 4 μl/min with an elution occurring with a 2%-12% solution B gradient and a spray operating on a 30 mm SilicaTip emitter.

A list of some of the MS transitions follows. Peptides are listed in the left column. The nomenclature “K. IATPR. G” indicates that the tryptic peptide is IATPR (SEQ ID NO: 16). The “.” in “K. IATPR. G” is used by the software program to mark the site of cleavage.

### Results

For predicting EYO, the best combination of tau biomarkers were phosphorylation occupancy at T205 (pT205/T205), MTBR-tau299/MTBR-tau354 ratio and MTBR-tau212 for all mutation carriers (MCs) (r²=0.6). pT205/T205, pT217/T217 and MTBR-tau212 were the best combination for asymptomatic MCs (r²=0.47), and pT205/T205 alone was most predictive for symptomatic MCs (r²=0.12). Baseline phosphorylation occupancies (pT217/T217, pT181/T181, pT153/T153, pT111/T111, pT205/T205, pS208/S208) and two MTBR ratios (MTBR-tau299/MTBR-tau354, MTBR-tau299/MTBR-tau282) were clustered together with amyloid pathology (PiB PET, CSF Aβ42/Aβ40 ratio), while MTBR-tau were more associated with total tau and neuronal injury/neuroinflammation biomarkers (YKL40, NGRN, VILIP1, SNAP25). Annual change of pT153/T153 had the highest correlation with annual change of PiB PET (−0.43) and CSF Aβ42/Aβ40 (0.67); whereas, annual change of pT217/T217, pT181/T181, total tau, pT153/T153, MTBR-3R, pT111/T111, pT231/T231, MTBR-tau275 and MTBR-tau299 were highly correlated with annual change of cognitive composite (r>0.5).

This work further highlights the diversity of soluble tau-related changes that occur with AD progression. Importantly, this study suggests that there are distinct phases in the evolution of tau-related pathological changes that track disease progression and specific non-tau biomarker changes. These findings could both help with understanding the role of tau in AD as well as an outcome of therapeutic trials. These findings also have utility in the diagnosis, prognosis and treatment of AD. As a non-limiting example, these findings may be used to determine whether a subject should receive additional diagnostic testing and/or select the appropriate test(s) (e.g., amyloid-based PET, tau-based PET, etc.). As another non-limiting example, these findings may be used to select a therapeutic agent, or class of therapeutic agents, for a subject that is tailored to the subject's disease stage and underlying disease pathology (as measured by the tau biomarkers).

TABLE 2
pTau by aMC, sMC and NC

| | | Mutation carriers | | Mutation non- | P- |
|---|---|---|---|---|---|
| Variable | n | Asymptomatic | Symptomatic | carriers | value |
| pT111/T111 | 150/78/141 | 4.16 ± 1.85 | 6.38 ± 2.14 | 3.09 ± 0.83 | <0.0001 |
| pT153/T153 | 151/78/141 | 0.90 ± 0.71 | 1.87 ± 0.93 | 0.41 ± 0.29 | <0.0001 |
| pS208/S208 | 137/76/125 | 0.58 ± 0.30 | 0.97 ± 0.34 | 0.36 ± 0.09 | <0.0001 |

Values are mean ± sd
P values are based on Kruskal-Wallis one way analysis of variance as data are skewed

TABLE 3
AUC of pTau species in classifying PiB PET
status (positive or negative) for MC

| Variable | AUC | 95% CI |
|---|---|---|
| pT217/T217 | 0.96 | (0.93, 0.99) |
| HVP/IGSL | 0.94 | (0.91, 0.97) |
| pT181/T181 | 0.94 | (0.90, 0.97) |
| pT153/T153 | 0.93 | (0.89, 0.98) |
| pT111/T111 | 0.92 | (0.88, 0.96) |
| pT231/T231 | 0.91 | (0.89, 0.96) |
| HVP/LDL | 0.91 | (0.87, 0.95) |
| MTBR299_317 | 0.91 | (0.87, 0.94) |
| pS208/S208 | 0.91 | (0.86, 0.96) |
| MTBR_3R | 0.90 | (0.86, 0.93) |
| pT205/T205 | 0.87 | (0.82, 0.91) |
| MTBR260_267 | 0.83 | (0.78, 0.88) |
| MTBR275_280 | 0.82 | (0.77, 0.87) |
| MTBR243_254 | 0.82 | (0.76, 0.87) |
| MTBR282_290 | 0.81 | (0.75, 0.86) |
| tTau | 0.79 | (0.74, 0.85) |
| MTBR212_211 | 0.77 | (0.71, 0.83) |
| MTBR354_369 | 0.75 | (0.68, 0.81) |
| pS202/S202 | 0.71 | (0.65, 0.78) |
| MTBR386_395 | 0.71 | (0.64, 0.78) |
| MTBR396_406 | 0.56 | (0.49, 0.64) |

TABLE 4
AUC of pTau species in classifying cognitive status
(CDR = 0 or CDR > 0) for MC

| Variable | AUC | 95% CI |
|---|---|---|
| pT205/T205 | 0.88 | (0.83, 0.93) |
| pT217/T217 | 0.84 | (0.79, 0.9) |
| HVP/IGSL | 0.83 | (0.78, 0.89) |
| pS208/S208 | 0.83 | (0.77, 0.88) |
| pT181/T181 | 0.82 | (0.76, 0.89) |
| pT111/T111 | 0.82 | (0.76, 0.87) |
| pT153/T153 | 0.82 | (0.76, 0.88) |
| HVP/LDL | 0.8 | (0.74, 0.86) |
| MTBR299_317 | 0.79 | (0.73, 0.85) |
| MTBR3R | 0.79 | (0.73, 0.85) |
| pT231/T231 | 0.79 | (0.73, 0.85) |
| tTau | 0.78 | (0.72, 0.85) |
| MTBR260_267 | 0.76 | (0.7, 0.82) |
| MTBR243_254 | 0.75 | (0.68, 0.81) |
| MTBR275_280 | 0.75 | (0.69, 0.82) |
| MTBR212_221 | 0.73 | (0.67, 0.8) |
| MTBR282_290 | 0.72 | (0.66, 0.79) |
| MTBR354_369 | 0.67 | (0.6, 0.74) |
| MTBR386_395 | 0.65 | (0.57, 0.72) |
| pS202/S202 | 0.59 | (0.52, 0.67) |
| MTBR396_406 | 0.47 | (0.39, 0.55) |

### Example 5

Background: Brain tau aggregation is a pathological hallmark of Alzheimer's disease (AD) and the tau microtubule binding region (MTBR) is the core of AD tau tangles. Recently, tau residues 243-254 in R1 (MTBR-tau243), 299-317 in R2 (MTBR-tau299), and 354-369 in R4 (MTBR-tau354) were identified as specifically enriched in sporadic AD brain tangles. Corresponding soluble MTBR species in the CSF increased with high correlations to clinical progression and tau pathology measured by tau-PET Because extracellular MTBR-tau was recently discovered in human AD, MTBR-tau has become a high priority target for antibodies targeting this region. In this study, we sought to determine when these MTBR-tau changes occurred and the relationship to clinical, cognitive and biomarker changes in the DIAN.

Method: A high precision method to quantify more than a dozen tau species in CSF was developed. To profile MTBR-tau, we conducted the immunoprecipitation using the anti-MTBR antibody, E2814 which is bi-epitopic to R2 and R4 and potential tau drug. We analyzed CSF from 227 mutation carriers (MCs) (152 asymptomatic, 77 symptomatic) and 141 non-carriers enrolled in the DIAN-observational cohort.

Result: CSF MTBR-tau specifically increased in MCs and changed at different times. The change of MTBR-tau299 in R2 occurred at an estimated year to symptom onset (EYO) of −22, close to the first detection of change in p-tau217 occupancy (EYO −21). The increase of MTBR-tau354 in R4 saturated in late clinical stages potentially due to the deposition into brain tangles. Notably, the ratio of MTBR-tau299/354 recapitulating tau pathophysiology highly correlated with p-tau217 occupancy, an early amyloid marker, in especially asymptomatic MCs (r=0.82), which indicates MTBR-tau and p-tau217 as biomarkers to assess anti-MTBR therapies in tau-PET negative populations.

Conclusion: We identified a soluble measure of tau tangles, MTBR species, with changes many years before symptom onset and continuing through symptomatic stages, suggesting early intervention can be implemented with anti-MTBR-tau antibodies, such as E2814. Finally, we utilized these results to design the tau next generation platform in the DIAN Trials Unit (DIAN-TU) E2814 drug arm to target soluble MTBR-tau in independent cohorts of asymptomatic and symptomatic carriers. In sporadic AD as well as DIAD, CSF “E2814-associated” MTBR increased in AD specifically. E2814-associated MTBR-tau profiles in whole AD continuum look similar between sAD and DIAD. Increase in early stage (preclinical stage) in sAD as well as DIAD (−20 EYO). The levels of E2814-associated MTBR decrease after AD onset (CDR>1, EYO>0). For MTBR-260 (late-R1), 270 (early R2), 282 (mid-R2) and 299 (R2-R3), E2814-associated MTBR-tau levels are well correlated with CX-levels. For MTBR-243 (R1-upstream) and 354 (R4), E2814-associated MTBR-tau levels are less correlated with CX-levels, potentially due to MTBR cleavage and the recruitment into tau-aggregation in brain. E2814-IP study revealed that the multiple cleavage sites are present in CSF MTBR-tau, e.g., 254(R1 upstream)˜260 (early R1) and 354(R4)-386 (C-term). Longitudinal assessment revealed the specific trajectories of MTBR-tau299 and 354 which increase before AD onset while they decrease after AD onset (->Reflection of brain tau aggregate). “E2814-associated MTBR-tau” in CSF can predicts cognitive measures in AD, in case the ratio of MTBR-299/354 is considered. “E2814-associated MTBR-tau” in CSF recapitulates tau pathology (tau-PET) at only “Early stage AD”, in case the ratio of MTBR-299/354 is considered.

1. A method for measuring time to dementia onset in a subject, the method comprising
(aᵢ) measuring phosphorylation occupancy at residue T205 of tau and measuring microtubule-binding region (MTBR)-tau299/MTBR-tau354 ratio in a blood sample or a cerebrospinal fluid (CSF) sample obtained from the subject, or
(aᵢᵢ) measuring phosphorylation occupancy at residue T205 of tau, measuring phosphorylation occupancy at residue T217 of tau, and measuring MTBR-tau212, in a blood sample or a CSF sample obtained from the subject;
(b) using the measurements of (aᵢ) or (aᵢᵢ) to calculate time to dementia onset, wherein time to dementia onset is time in years to a Clinical Dementia Rating (CDR) greater than zero; and
(c) treating the subject with medical care, wherein the medical care is selected from
(i) a prophylactic or preventative measure;
(ii) administration of a diagnostic agent: or
(iii) administration of a therapeutic agent.
wherein the subject
is without a cognitive or behavioral symptom of Alzheimer's disease; or
has one or more cognitive or behavioral symptom of Alzheimer's disease.

2. A method for measuring time to dementia onset in a subject, the method comprising
(a) processing a blood sample or a cerebrospinal fluid (CSF) sample from the subject to obtain a first population of tau species and a depleted sample, and then processing the depleted sample to obtain a second population of tau species,
wherein the first population of tau species is enriched for N-terminal tau and/or mid-domain tau, and the second population of enriched tau species is enriched for microtubule-binding region (MTBR)-tau;
(bᵢ) measuring phosphorylation occupancy at residue T205 of tau in the first population of tau species and measuring MTBR-tau299/MTBR-tau354 ratio in the second population of tau species;
(bᵢᵢ) measuring phosphorylation occupancy at residue T205 of tau and measuring phosphorylation occupancy at residue T217 of tau in the first population of tau species, and measuring MTBR-tau212 in the second population of tau species;
(c) calculating time to dementia onset using the measurements of (bᵢ) or (bᵢᵢ), wherein time to dementia onset is time in years to a Clinical Dementia Rating (CDR) greater than zero; and
(d) treating the subject with medical care, wherein the medical care is selected from
(i) a prophylactic or preventative measure;
(ii) administration of a diagnostic agent; or
(iii) administration of a therapeutic agent.
wherein the subject
is without a cognitive or behavioral symptom of Alzheimer's disease; or
has one or more cognitive or behavioral symptom of Alzheimer's disease.

3. The method of claim 2, wherein the processing of the blood sample or the CSF sample from the subject to obtain the first population of enriched tau species and the depleted sample comprises
contacting the blood sample or the CSF sample with an epitope-binding agent that specifically binds to an epitope within the N-terminus of tau, or
contacting the blood sample or the CSF sample with an epitope-binding agent that specifically binds to an epitope within the mid-domain of tau, or
contacting the blood sample or the CSF sample with a first epitope-binding agent that specifically binds to an epitope within the N-terminus of tau and with a second epitope-binding agent that specifically binds to an epitope within the mid-domain of tau, wherein the first and second epitope-binding agents are used sequentially or at the same time.

4. The method of claim 2, wherein the processing of the depleted sample to obtain the second population of enriched tau species comprises
performing a chemical extraction step to enrich for MTBR-tau species;
or
contacting the depleted sample with an epitope-binding agent that specifically binds to at least one epitope within the MTBR of tau.

5. The method of claim 2, wherein
processing the blood sample or the CSF sample from the subject to obtain the first population of enriched tau species and the depleted sample, comprising
contacting the blood sample or the CSF sample with an epitope-binding agent the specifically binds to an epitope within the N-terminus of tau, or
contacting the blood sample or the CSF sample with an epitope-binding agent the specifically binds to an epitope within the mid-domain of tau, or
contacting the blood sample or the CSF sample with a first epitope-binding agent that specifically binds to an epitope within the N-terminus of tau and with a second epitope-binding agent that specifically binds to an epitope within the mid-domain of tau, wherein the first and second epitope-binding agents are used sequentially or at the same time; and
processing the depleted sample to obtain a second population of enriched tau species, comprising
performing a chemical extraction step to enrich for MTBR-tau species; or
contacting the depleted sample with an epitope-binding agent that specifically binds to at least one epitope within the MTBR of tau.

6. (canceled)

7. A method for staging a subject's disease progression or brain pathology, the method comprising measuring time to dementia onset in the subject according to the method of claim 1 and staging the subject's disease progression or brain pathology based on the calculated time to dementia onset.

8. A method for staging a subject's disease progression or brain pathology, the method comprising measuring time to dementia onset in the subject according to the method of claim 2 and staging the subject's disease progression or brain pathology based on the calculated time to dementia onset.

9. The method of claim 1, wherein
the prophylactic or preventative measure is selected from
(i) preventing or slowing an undesired physiological change in the subject;
(ii) stabilizing the state of disease in the subject;
(iii) delaying or slowing disease progression in the subject;
(iv) improving memory loss, mood, or behavior in the subject; or
(v) a dietary supplement;
the diagnostic agent is selected from
(vi) a tau PET tracer: or
(vii) an Aβ PET tracer: or
the therapeutic agent is selected from an agent that
(viii) decreases Aβ production in the subject;
(ix) prevents or antagonizes Aβ aggregation in the subject;
(x) increases brain Aβ clearance in the subject;
(xi) alters tau phosphorylation patterns in the subject;
(xii) prevents or antagonizes tau aggregation in the subject: or
(xiii) increases neurofibrillary tangle (NFT) clearance in the subject.

10. The method of claim 2, wherein
the prophylactic or preventative measure is selected from
(i) preventing or slowing an undesired physiological change in the subject;
(ii) stabilizing the state of disease in the subject;
(iii) delaying or slowing disease progression in the subject;
(iv) improving memory loss, mood, or behavior in the subject: or
(v) a dietary supplement.
the diagnostic agent is selected from
(vi) a tau PET tracer: or
(vii) an Aβ PET tracer; or
the therapeutic agent is selected from an agent that
(viii) decreases Aβ production in the subject;
(ix) prevents or antagonizes Aβ aggregation in the subject;
(x) increases brain Aβ clearance in the subject;
(xi) alters tau phosphorylation patterns in the subject;
(xii) prevents or antagonizes tau aggregation in the subject: or
(xiii) increases neurofibrillary tangle (NFT) clearance in the subject.

11. The method of claim 9, wherein the diagnostic agent is
(i) a tau PET tracer selected from flortaucipir, MK-6240, PI-2620, RO-948, GPT1, JNJ-067, JNJ-64349311, APN-1607, SNFT-1, PM-PBB3, AV1451, THK5351, or THK5317; or
(ii) an Aβ PET tracer selected from FDDNP, AV-45, GE067, BAY94-9172, PI, GDF, NaF, p5+14, florbetapir, florbetaben, or flutemetamol.

12. The method of claim 10, wherein the diagnostic agent is
(i) a tau PET tracer selected from flortaucipir, MK-6240, PI-2620, RO-948, GPT1, JNJ-067, JNJ-64349311, APN-1607, SNFT-1, PM-PBB3, AV1451, THK5351, or THK5317: or
(ii) a Aβ PET tracer selected from FDDNP, AV-45, GE067, BAY94-9172, PI, GDF, NaF, p5+14, florbetapir, florbetaben, or flutemetamol.

13. The method of claim 9, wherein the therapeutic agent
(i) decreases Aβ production, prevents or antagonizes Aβ aggregation, or increases brain Aβ clearance in the subject, and is selected from a gamma-secretase inhibitor, beta-secretase inhibitor, passive immunotherapy, or an active immunotherapy, or
(ii) alters tau phosphorylation patterns, prevents or antagonizes tau aggregation, or increases NFT clearance in the subject, and is selected from a tau protein aggregation inhibitor, kinase inhibitor, phosphatase activator, passive immunotherapy, or an active immunotherapy.

14. The method of claim 13, wherein the therapeutic agent is selected from
(i) AZD3283; MK-8931; tarenflurbil; semagacestat; solanezumab; bapineuzumab: aducanumab; B11181181; RG7129; LY281137; LY2886721; E2509; AZD3293; CNP520; JNJ-54861911; verubecestat; avagacestat; EVP-0962; NIC5-15; pinitol; AN-1792; CAD106; vanutide cridificar; bapineuzumab; AAB-003; GSK933776; solanezumab; crenezumab; gantenerumab: BAN2401; aducanumab; an anti-APOE antibody, or NPT088, or
(ii) lithium: valproic acid: methylthioninium; Epothilone D; TPI287; Rember; TRxo237; AADvac-1; ACI-35; memantine; sodium selenite; tideglusib; lithium chloride; salsalte; MK-8719; LMTX; curcumin; NAP; TPI 287; BPN14770; Ionis MAPT Rx; nilotinib; nicotinamide; AZD0530: LY3303560: ABBV-8E12; BIIB092; 637733657; RO7105705; NPT008; methylene blue; or a morphomer.

15. The method of claim 10, wherein the therapeutic agent
(i) decreases Aβ production, prevents or antagonizes Aβ aggregation, or increases brain Aβ clearance in the subject, and is selected from a gamma-secretase inhibitor, beta-secretase inhibitor, passive immunotherapy, or an active immunotherapy, or
(ii) alters tau phosphorylation patterns, prevents or antagonizes tau aggregation, or increases NFT clearance in the subject, and is selected from a tau protein aggregation inhibitor, kinase inhibitor, phosphatase activator, passive immunotherapy, or an active immunotherapy.

16. The method of claim 15, wherein the therapeutic agent is selected from
(i) AZD3283; MK-8931; tarenflurbil; semagacestat; solanezumab; bapineuzumab; aducanumab; BI11181181; RG7129; LY281137; LY2886721; E2509; AZD3293; CNP520; JNJ-54861911; verubecestat; avagacestat; EVP-0962; NIC5-15; pinitol; AN-1792; CAD106; vanutide cridificar; bapineuzumab; AAB-003; GSK933776; solanezumab; crenezumab; gantenerumab; BAN2401; aducanumab; an anti-APOE antibody, or NPT088, or
(ii) lithium; valproic acid; methylthioninium; Epothilone D; TP1287; Rember; TRxo237; AADvac-L; ACI-35; memantine; sodium selenite; tideglusib; lithium chloride; salsalte; MK-8719; LMTX; curcumin; NAP; TPI 287; BPN14770; Ionis MAPT Rx; nilotinib; nicotinamide; AZD0530; LY3303560; ABBV-8E12; BIIB092; 637733657; R07105705; NPT008; methylene blue; or a morphomer.

17. (canceled)

18. (canceled)

19. The method of claim 3, wherein
the epitope-binding agent that specifically binds to an epitope within the N-terminus of tau is HJ8.5 or another epitope-binding agent that specifically binds the same epitope as HJ8.5, or
the epitope-binding agent that specifically binds to an epitope within the mid-domain of tau is Tau1 or another epitope-binding agent that specifically binds the same epitope as Tau1.

20. The method of claim 4, wherein
the epitope-binding agent that specifically binds to at least one epitope within the MTBR of tau
is selected from 77G7, RD3, RD4, UCB 1017, or PT76 described in Vandermeeren et al., J Alzheimers Dis, 2018, 65:265-281, or 7G6 described in Roberts et al., Acta Neuropathol Commun, 2020, 8: 13, or antigen-binding fragments of 77G7, RD3, RD4, UCB 1017, PT76, or 7G6, or other epitope-binding agents that specifically bind the same epitopes as 77G7, RD3, RD4, UCB 1017, PT76, or 7G6.

21.-34. (canceled)

35. A method for measuring change in cognition in a subject, the method comprising
(a) measuring phosphorylation occupancy at one or more residue of tau selected from T111, T153, T181, T217 and T231 in a blood sample or a cerebrospinal fluid (CSF) sample obtained from the subject, and
measuring at least one of microtubule-binding region (MTBR)-tau275, MTBR-tau299, and MTBR-3R in a blood sample or a CSF sample obtained from the subject, and
(b) using the measurements of (a) to calculate a change in cognition.

36. The method of claim 35, wherein the change in cognition is equivalent to the change in cognition measured by cognitive composite score comprising the delayed recall score from the International Shopping List Test, the Logical Memory delayed recall score from the Wechsler Memory Scale-Revised, the Digit Symbol Coding test total score from the Wechsler Adult Intelligence Scale-Revised, and the MMSE total score.

37. The method of claim 35, wherein the subject has a CDR of zero.

38. The method of claim 35, wherein the subject has a CDR greater than or equal to 0.5.

39. A method for evaluating the effectiveness of a therapeutic agent in a subject, comprising measuring the change in cognition in the subject according to claim 35, and determining the therapeutic to be effective if cognition in the subject is improved after administration of the therapeutic agent to the subject.


SEQUENCE LISTING (21 sequences)

SEQ ID NO: 1 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 8 aa, Artificial Sequence
  Synthesized
IGSTENLK

SEQ ID NO: 3 - protein, 12 aa, Artificial Sequence
  Synthesized
LQTAPVPMPDLK

SEQ ID NO: 4 - protein, 6 aa, Artificial Sequence
  Synthesized
VQIINK

SEQ ID NO: 5 - protein, 9 aa, Artificial Sequence
  Synthesized
LDLSNVQSK

SEQ ID NO: 6 - protein, 19 aa, Artificial Sequence
  Synthesized
HVPGGGSVQIVYKPVDLSK

SEQ ID NO: 7 - protein, 16 aa, Artificial Sequence
  Synthesized
IGSLDNITHVPGGGNK

SEQ ID NO: 8 - protein, 15 aa, Artificial Sequence
  Synthesized
IGSLDNITHVPGGGN

SEQ ID NO: 9 - protein, 12 aa, Artificial Sequence
  Synthesized
VQIVYKPVDLSK

SEQ ID NO: 10 - protein, 10 aa, Artificial Sequence
  Synthesized
TPPSSGEPPK

SEQ ID NO: 11 - protein, 18 aa, Artificial Sequence
  Synthesized
QEFEVMEDHAGTYGLGDR

SEQ ID NO: 12 - protein, 20 aa, Artificial Sequence
  Synthesized
DQGGYTMHQDQEGDTDAGLK

SEQ ID NO: 13 - protein, 23 aa, Artificial Sequence
  Synthesized
ESPLQTPTEDGSEEPGSETSDAK

SEQ ID NO: 14 - protein, 20 aa, Artificial Sequence
  Synthesized
STPTAEDVTAPLVDEGAPGK

SEQ ID NO: 15 - protein, 39 aa, Artificial Sequence
  Synthesized
QAAAQPHTEIPEGTTAEEAGIGDTPSLEDEAAGHVTQAR

SEQ ID NO: 16 - protein, 5 aa, Artificial Sequence
  Synthesized
IATPR

SEQ ID NO: 17 - protein, 15 aa, Artificial Sequence
  Synthesized
SGYSSPGSPGTPGSR

SEQ ID NO: 18 - protein, 10 aa, Artificial Sequence
  Synthesized
TPSLPTPPTR

SEQ ID NO: 19 - protein, 5 aa, Artificial Sequence
  Synthesized
VAVVR

SEQ ID NO: 20 - protein, 10 aa, Artificial Sequence
  Synthesized
TDHGAEIVYK

SEQ ID NO: 21 - protein, 11 aa, Artificial Sequence
  Synthesized
SPVVSGDTSPR